(19) United States
(12) Patent Application Publication
(10) Pub. No.: US 2002/0049316 A1
(43) Pub. Date: Apr. 25, 2002

(54) Protease inhibitors

(76) Inventors: Stacie Marie Halbert, Harleysville, PA (US); Evelyne Michaud, Norristown, PA (US); Scott Kevin Thompson, Phoenixville, PA (US); Daniel Frank Veber, Ambler, PA (US)

Correspondence Address: GLAXOSMITHKLINE, Corporate Intellectual Property - UW2220, P.O. Box 1539, King of Prussia, PA, 19406-0939, US

(21) Appl. No.: 10/022,713

(22) Filed: Dec. 17, 2001

Related U.S. Application Data
(63) Continuation of application No. 09/423,059, filed on Oct. 29, 1999, now abandoned.
Application No. 09/423,059 is a 371 of international application No. PCT/US98/08740, filed on Apr. 29, 1998.
(60) Provisional application No. 60/045,067, filed on Apr. 29, 1997.

(51) Int. Cl. C07D 417/02; C07D 413/02; C 07D 4 3/02

(52) U.S. Cl. 540/603; 544/60; 544/137; 544/139; 544/133; 544/238; 544/333; 544/387; 544/369; 544/370; 546/209; 546/210; 546/141; 546/153

(57) ABSTRACT

The present invention provides compounds of formula (I) which inhibit proteases, including cathepsin K, pharmaceutical compositions of such compounds, and methods for treating diseases of excessive bone loss or cartilage or matrix degradation, including osteoporosis; gingival disease including gingivitis and periodontitis; arthritis, more specifically, osteoarthritis and rheumatoid arthritis; Paget's disease; hypercalcemia or malignancy; and metabolic bone disease therewith.

### FIELD OF THE INVENTION

This invention relates in general to heterocycleketohydrazide protease inhibitors, particularly such inhibitors of cysteine and serine proteases, more particularly compounds which inhibit cysteine proteases, even more particularly compounds which inhibit cysteine proteases of the papain superfamily, yet more particularly compounds which inhibit cysteine proteases of the cathepsin family, most particularly compounds which inhibit cathepsin K. Such compounds are particularly useful for treating diseases in which cysteine proteases are implicated, especially diseases of excessive bone or cartilage loss, e.g., osteoporosis, periodontitis, and arthritis.

### BACKGROUND OF THE INVENTION

Bone is composed of a protein matrix in which spindle- or plate-shaped crystals of hydroxyapatite are incorporated. Type I Collagen represents the major structural protein of bone comprising approximately 90% of the structural protein. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodeling at discrete foci throughout life. These foci, or remodeling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

Several published studies have demonstrated that inhibitors of cysteine proteases are effective at inhibiting osteoclast-mediated bone resorption, and indicate an essential role for a cysteine proteases in bone resorption. For example, Delaisse, et al., Biochem. J., 1980, 192, 365, disclose a series of protease inhibitors in a mouse bone organ culture system and suggest that inhibitors of cysteine proteases (e.g., leupeptin, Z-Phe-Ala-CHN₂) prevent bone resorption, while serine protease inhibitors were ineffective. Delaisse, et al., Biochem. Biophys. Res. Commun., 1984, 125, 441, disclose that E-64 and leupeptin are also effective at preventing bone resorption in vivo, as measured by acute changes in serum calcium in rats on calcium deficient diets. Lerner, et al., J. Bone Min. Res., 1992, 7, 433, disclose that cystatin, an endogenous cysteine protease inhibitor, inhibits PTH stimulated bone resorption in mouse calvariae. Other studies, such as by Delaisse, et al., Bone, 1987, 8, 305, Hill, et al., J. Cell. Biochem., 1994, 56, 118, and Everts, et al., J. Cell. Physiol., 1992, 150, 221, also report a correlation between inhibition of cysteine protease activity and bone resorption. Tezuka, et al., J. Biol. Chem., 1994, 269, 1106, Inaoka, et al., Biochem. Biophys. Res. Commun., 1995, 206, 89 and Shi, et al., FEBS Lett., 1995, 357, 129 disclose that under normal conditions cathepsin K (which has also been called cathepsin O), a cysteine protease, is abundantly expressed in osteoclasts and may be the major cysteine protease present in these cells.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

Palmer, et al., J. Med. Chem., 1995, 38, 3193, disclose certain vinyl sulfones which irreversibly inhibit cysteine proteases, such as the cathepsins B, L, S, O2 and cruzain. Other classes of compounds, such as aldehydes, nitrites, a-ketocarbonyl compounds, halomethyl ketones, diazomethyl ketones, (acyloxy)methyl ketones, ketomethylsulfonium salts and epoxy succinyl compounds have also been reported to inhibit cysteine proteases. The synthesis of azatides (polyacylhydrazides) as peptide mimetics has recently been disclosed by Han and Janda, J. Am. Chem. Soc. 1996, 118, 2539.

The synthesis of N-phenyl-N′-(2-phenyloxazol-4-ylcarbonyl)hydrazide, as well as its N-(2,4dinitrophenyl) derivative, have been described in Afridi, A., et al., J. Chem. Soc, Perkin Trans. I, 1976, 3, 315-20. Benko, A., et al., Justus Liebigs Ann. Chem., 1968, 717, 148-53 describes the preparation of N-(4ethoxycarbonylthiazol-2-yl)-N′-[2-(4-pyridinyl)thiazol-4-ylcarbonyl]hydrazide.

Thus, a structurally diverse variety of cysteine protease inhibitors have been identified. However, these known inhibitors are not considered suitable for use as therapeutic agents in animals, especially humans, because they suffer from various shortcomings. These shortcomings include lack of selectivity, cytotoxicity, poor solubility, and overly rapid plasma clearance. A need therefore exists for methods of treating diseases caused by pathological levels of proteases, especially cysteine proteases, including cathepsins, especially cathepsin K, and for novel inhibitor compounds useful in such methods.

We have now discovered a novel class of heterocycleloketohydrazide compounds which are protease inhibitors, most particularly of cathepsin K.

### SUMMARY OF THE INVENTION

An object of the present invention is to provide heterocycleketohydrazide protease inhibitors, particularly such inhibitors of cysteine and serine proteases, more particularly such compounds which inhibit cysteine proteases, even more particularly such compounds which inhibit cysteine proteases of the papain superfamily, yet more particularly such compounds which inhibit cysteine proteases of the cathepsin family, most particularly such compounds which inhibit cathepsin K, and which are useful for treating diseases which may be therapeutically modified by altering the activity of such proteases.

Accordingly, in the first aspect, this invention provides a compound according to Formula I.

In another aspect, this invention provides a pharmaceutical composition comprising a compound according to Formula I and a pharmaceutically acceptable carrier, diluent or excipient.

In yet another aspect, this invention provides intermediates useful in the preparation of the compounds of Formula I.

In still another aspect, this invention provides methods of treating diseases in which the disease pathology may be therapeutically modified by inhibiting proteases, particularly cysteine and serine proteases, more particularly cysteine proteases, even more particularly cysteine proteases of the papain superfamily, yet more particularly cysteine proteases of the cathepsin family, most particularly cathepsin K.

In a particular aspect, the compounds of this invention are especially useful for treating diseases characterized by bone loss, such as osteoporosis and gingival diseases, such as gingivitis and periodontitis, or by excessive cartilage or matrix degradation, such as osteoarthritis and rheumatoid arthritis.

### DETAILED DESCRIPTION OF THE INVENTION

The present invention provides compounds of Formula I:

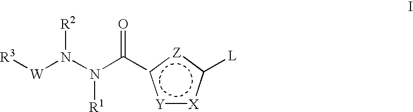

wherein:

L is C₂₋₆alkyl, Ar—C₀₋₆alkyl, Het—C₀₋₆alkyl, CH(R⁴)NR⁵R⁶, CH(R⁴)Ar, CH(R⁴)OAr′, or NR⁴R⁷;

Ar is phenyl or naphthyl, optionally independently substituted by one or more of Ph—C₀₋₆alkyl, Het—C₀₋₆alkyl, C₁₋₆alkyl, C₁₋₆alkoxy, Ph—C₀₋₆alkoxy, Het—C₀₋₆alkoxy, OH, (CH₂)₁₋₆NR⁸R⁹, O(CH₂)₁₋₆NR⁸R⁹, CO₂R′, or halogen. Two C₁₋₆alkyl groups may be combined to form a 5-7 membered ring, saturated or unsaturated, fused onto the Ar ring. Ph may be optionally substituted with one or more of C₁₋₆alkyl, C₁₋₆alkoxy, OH, (CH₂)₁₋₆NR⁸R⁹, O(CH₂)₁₋₆NR⁸R⁹, CO₂R′, or halogen.

Ar′ is phenyl or naphthyl, optionally independently substituted by one or more of Ph—C₀₋₆alkyl, Het—C₀₋₆alkyl, C₁₋₆alkyl, C₁₋₆alkoxy, Ph—C₀₋₆alkoxy, Het—C₀₋₆alkoxy, OH, (CH₂)₁₋₆NR⁸R⁹, O(CH₂)₁₋₆NR⁸R⁹, or halogen. Ph may be optionally substituted with one or more of C₁₋₆alkyl, C₁₋₆alkoxy, OH, (CH₂)₁₋₆NR⁸R⁹, O(CH₂)₁₋₆NR⁸R⁹, CO₂R′, or halogen. Two C₁₋₆alkyl groups may be combined to form a 5-7 membered ring, saturated or unsaturated, fused onto the Ar′ ring.

Het is a stable 5- to 7-membered monocyclic or a stable 7- to 10-membered bicyclic heterocyclic ring, which is either saturated or unsaturated, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from the group consisting of Ph—C₀₋₆alkyl, Het—C₀₋₆ alkyl, C₁₋₆alkyl, C₁₋₆alkoxy, Ph—C₀₋₆alkoxy, Het—C₀₋₆alkoxy, OH, (CH₂)₁₋₆ NR⁸R⁹, O(CH₂)₁₋₆NR⁸R⁹, CO₂R′. Two C₁₋₆alkyl groups may be combined to form a 5-7 membered ring, saturated or unsaturated, fused onto the Het ring. Ph may be optionally substituted with one or more of C₁₋₆alkyl, C₁₋₆alkoxy, OH, (CH₂)₁₋₆NR⁸R⁹, O(CH₂)₁₋₆NR⁸R⁹, CO₂R′, or halogen. Preferably, such heterocycles are selected from the group consisting of the piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, triazolyl, tetrazolyl, pyridyl, pyrazinyl, pryidazinyl, pyrimidinyl, triazinyl, tetrazinyl, oxazolidinyl, oxazolinyl, oxazolyl, isothiazolyl, isoxazolyl, morpholinyl, thiazolidinyl, thiazolinyl, thiazolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, thiamorpholinyl sulfoxide, thiamorpholinyl sulfone, thiadiazolyl, and oxadiazolyl rings.

W is C(O) or SO₂;

X, Y, and Z are independently N, O, S or CR¹⁰, provided that at least two of X, Y and Z are heteroatoms and at least one of X, Y and Z is N, or one of X, Y and Z is C═N, C═C or N═N and the other two are CR¹⁰ or N, further provided that at least two of X, Y and Z are N;

[symbol] indicates a single or double bond in the five-membered heterocycle;

R′, R¹, R², R⁵, R⁸, R⁹, R¹⁰, and R¹² are independently H, C₁₋₆alkyl, C₂₋₆alkenyl, Ar—C₀₋₆alkyl, or Het—C₀₋₆alkyl;

R³ is C₃₋₆alkyl, Ar, Het, CH(R¹¹)Ar, CH(R¹¹)OAr, NR¹¹R¹², CH(R¹¹)NR¹²R¹³; or

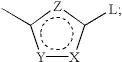

R⁴, R¹¹, and R¹⁵ are independently H, C₁₋₆alkyl, C₂₋₆alkenyl, C₃₋₆cycloalkyl-C₀₋₆-alkyl, Ar—C₀₋₆alkyl, or Het—C₀₋₆alkyl;

R⁷ is C₁₋₆alkyl, C₁₋₆alkenyl, C₃₋₆cycloalkyl-C₀₋₆-alkyl, Ar—C₀₋₆alkyl, or Het—C₀₋₆alkyl; R⁴ and R⁷ may be combined to form a 3-7 membered monocyclic or 7-10-membered bicyclic carbocyclic or heterocyclic ring, optionally independently substituted with 1-4 of C₁₋₆alkyl, Ar—C₀₋₆alkyl, Het—C₀₋₆alkyl, C₁₋₆alkoxy, Ar—C₀₋₆alkoxy, Het—C₀₋₆alkoxy, OH, (CH₂)₁₋₆NR⁸R⁹, or O(CH₂)₁₋₆NR⁸R⁹;

R⁶ and R¹³ are R¹⁴, R¹⁴C(O), R¹⁴C(S), R¹⁴OC(O), or R¹⁴OC(O)NR⁹CH(R¹⁵)(CO); and

R¹⁴ is C₁₋₆alkyl, C₂₋₆alkenyl, Ar—C₀₋₆alkyl, or Het—C₀₋₆alkyl.

Compounds of Formula I wherein Z=N, X=S, and Y=CH (thiazolo) are preferred. More preferred are such compounds wherein W is C(O). Even more preferred are such compounds wherein R¹ and R² are H.

Yet more preferred are such compounds wherein R³ is:

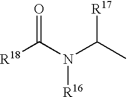

wherein:

R¹⁶ is H or C₁₋₆alkyl, preferably H or Me;

R¹⁷ is C₁₋₆alkyl, C₂₋₆alkenyl, and C₃₋₁₁cycloalkyl-C₁₋₆alkyl, preferably n-propyl, iso-propyl, iso-pentyl, tert-butylmethyl, cyclopropylmethyl, iso-butyl, n-butyl, or allyl; and

R¹⁸ is C₃₋₆alkyl, OC₃₋₆alkyl, Ar, Het, O(CH₂)₀₋₃Ar, or O(CH₂)₀₋₃Het, preferably 2-pyridinylmethoxy, 3-pyridinylmethoxy, 4-pyridinylmethoxy, tert-butoxy, 2-pyridinyl, 3-pyridinyl, 4-pyridinyl, 2-pyrazinyl, 4-tert-butoxycarbonylbenzyloxy, 4-carboxybenzyloxy, 3-tert-butoxycarbonylbenzyloxy, 3-carboxybenzyloxy, 2-methyl-3-pyridinylmethoxy, 6-methyl-3-pyridinylmethoxy, benzyloxy, 2-quinolino, 3-quinolino, 4-quinolino, 5-quinolino, 6-quinolino, 7-quinolino, 8-quinolino, 1-isoquinolino, 3-isoquinolino, piperidinyl, 4-methylpiperidinyl, 4-methylimidazol-5-yl, N-benzyl-pyrrolidinyl, N-methyl-pyrrolidinyl, 1-benzyl-5-methylimidazol-4-yl, 1-piperazinyl; 3-(2-pyridyl)benzyl, 2-methyl-3-pyridinyl, 2-methyl-4-pyridinyl, 6-methyl-3-pyridinyl, 4-dimethylaminobenzyloxy, 4-(4-morpholinomethyl)phenyl, 5-hydroxymethylimidazol-4-yl, 5-butyl-2-pyridinyl, 4-fluorophenyl, 3,4-difluorophenyl, 2-(1,8-naphthyridinyl), or 3,4-dimethoxyphenyl.

Also yet more preferred are compounds of Formula I wherein Z═N, X═S, and Y═CH (thiazolo), W is C(O), R¹ and R² are H, and wherein L is 4-(cis-2,6-dimethyl)-4-morpholinyl, N-cyclopropylmethyl-N-(2-methylpropyl)amino, 4-methyl-1-naphthyl, N-methyl-N-(2-methylpropyl)amino, 1-naphthyl, 5-acenaphthyl, N-cyclopropyl-N-cyclopropylmethylamino, N,N-bis-(2-methylpropyl)amino, 1-(1,2,3,4-tetrahydroquinolino, N-cyclopropylmethyl-N-propylamino, N-(2-methylpropyl)-N-phenylamino, 2-methoxy-1-naphthyl, 2-benzyloxyphenyl, 2-benzyloxy-1-naphthyl, 9-phenanthrenyl, 9-anthracenyl, phenyl, 2-(4-tert-butoxycarbonyl)benzyloxyphenyl, 2-(4-carboxybenzyloxy)phenyl, N-cyclopropylamino, 8-quinolino, N,N-bis-(cyclopropylmethyl)amino, 4-(2,2-dimethylaminoethoxy)-1-naphthyl, or 1-(N-benzyloxycarbonylamino)-3-methylbutyl.

The following compounds are particularly preferred embodiments of the present invention:

N-[2-(cis-2,6-dimethyl-4-morpholino)thiazol-4-ylcarbonyl]-N′-[N-(4-pyridinylmethoxycarbonyl)-L-leucinyl]hydrazide;

N-[2-[N-cyclopropylmethyl-N-(2-methylpropyl)amino]thiazol-4-ylcarbonyl]-N′-[N-(4-pyridinylmethoxycarbonyl)-L-leucinyl]hydrazide;

N-[2-(4-methyl-1-naphthyl)thiazol-4-ylcarbonyl]-N′-[N-(4-pyridinylmethoxycarbonyl)-L-leucinyl]hydrazide;

N-[2-[N-methyl-N-(2-methylpropyl)amino]thiazol-4-ylcarbonyl]-N′-[N-methyl-N-(4-pyridinylmethoxycarbonyl)-L-leucinyl]hydrazide;

N-[2-(1-naphthyl)thiazol-4-ylcarbonyl]-N′-[N-(3-pyridinylmethoxycarbonyl)-L-leucinyl]hydrazide;

N-[2-(1-naphthyl)thiazol-4-ylcarbonyl]-N′-[N-(2-pyridinylmethoxycarbonyl)-L-leucinyl]hydrazide;

N-[2-(5-acenaphthyl)thiazol-4-ylcarbonyl]-N′-[N-(4-pyridinylmethoxycarbonyl)-L-leucinyl]hydrazide;

N-[2-[N-cyclopropylmethyl-N-(2-methylpropyl)amino]thiazol-4-ylcarbonyl]-N′-[N-methyl-N-(4-pyridinylmethoxycarbonyl)-L-leucinyl]hydrazide;

N-[2-(N-cyclopropyl-N-cyclopropylmethylamino)thiazol-4-ylcarbonyl]-N′-[N-(4-pyridinylmethoxycarbonyl)-L-leucinyl]hydrazide;

N-[2-[N-cyclopropylmethyl-N-(2-methylpropyl)amino]thiazol-4-ylcarbonyl]-N′-[N-(3-pyridinylmethoxycarbonyl)-L-leucinyl]hydrazide;

N-[2-[N-cyclopropylmethyl-N-(2-methylpropyl)amino]thiazolyl-4-ylcarbonyl]-N′-[N-(2-pyridinylmethoxycarbonyl)-L-leucinyl]hydrazide;

N-(2-[N-cyclopropylmethyl-N-(2-methylpropyl)amino]thiazol-4-ylcarbonyl]-N′-[N-methyl-N-(3-pyridinylmethoxycarbonyl)-L-leucinyl]hydrazide;

N-[2-(N-cyclopropyl-N-cyclopropylmethylamino)thiazol-4-ylcarbonyl]-N′-[N-(3-pyridinylmethoxycarbonyl)-L-leucinyl]hydrazide;

N-[2-(N-cyclopropyl-N-cyclopropylmethylamino)thiazol-4-ylcarbonyl]-N′-[N-methyl-N-(4-pyridinylmethoxycarbonyl)-L-leucinyl]hydrazide;

N-[2-[N,N-bis-(2-methylpropyl)amino]thiazol-4-ylcarbonyl]-N′-[N-(2-pyridinylmethoxycarbonyl)-L-leucinyl]hydrazide;

N-[N-(4-pyridinylmethoxycarbonyl)-L-leucinyl]-N′-[2-[1-(1,2,3,4-tetrahydroquinolino)]thiazol-4-ylcarbonyl]hydrazide;

N-[2-[N-methyl-N-(2-methylpropyl)amino]thiazol-4-ylcarbonyl]-N′-[4-methyl-2-(3-phenyl)phenylpent-4-enoyl]hydrazide;

N-[2-[N,N-bis-(2-methylpropyl)amino]thiazol-4-ylcarbonyl]-N′-[N-methyl-N-(3-pyridinylmethoxycarbonyl)-L-leucinyl]hydrazide;

N-[2-(N-cyclopropyl-N-cyclopropylmethylamino)thiazol-4-ylcarbonyl]-N′-[N-methyl-N-(3-pyridinylmethoxycarbonyl)-L-leucinyl]hydrazide;

N-[2-(N-cyclopropylmethyl-N-propylamino)thiazol-4-ylcarbonyl]-N′-[N-(3-pyridinylmethoxycarbonyl)-L-leucinyl]hydrazide;

N-[2-[N-methyl-N-(2-methylpropyl)amino]thiazol-4-ylcarbonyl]-N′-[4-methyl-2-(3-phenyl)phenylpentanoyl]hydrazide;

N-[N-(2-methylpropyl)-N-(3-phenylphenyl)carbamoyl]-N′-[2-(1-naphthyl)thiazol-4-ylcarbonyl]hydrazide;

N-[4-methyl-2-(3-phenyl)phenylpentanoyl]-N′-[2-(1-naphthyl)thiazol-4-ylcarbonyl]hydrazide;

N-[4-methyl-2-(3-phenyl)phenylpentanoyl]-N′-[2-[N-(2-methylpropyl)-N-phenylamino]thiazol-4-ylcarbonyl]hydrazide;

N[2-(2-methoxy-1-naphthyl)thiazol-4-ylcarbonyl]-N′-[N-(4-pyridinylmethoxycarbonyl)-L-leucinyl]hydrazide;

N-[2-(2-benzyloxyphenyl)thiazol-4-ylcarbonyl]-N′-[4-methyl-2-(3-phenyl)phenylpentanoyl]hydrazide;

N-[2-(2-benzyloxy-1-naphthyl)thiazol-4-ylcarbonyl]-N′-[N-(4-pyridinylmethoxycarbonyl)-L-leucinyl]hydrazide;

N-[2-[N,N-bis-(2-methylpropyl)amino]thiazol-4-ylcarbonyl]-N′-[N-methyl-N-(2-pyridinylmethoxycarbonyl)-L-leucinyl]hydrazide;

N-[2-(9-phenanthrenyl)thiazol-4-ylcarbonyl]-N′-[N-(4-pyridinylmethoxycarbonyl)-L-leucinyl]hydrazide;

N-[2-(9-anthracenyl)thiazol-4-ylcarbonyl]-N′-[N-(4-pyridinylmethoxycarbonyl)-L-leucinyl]hydrazide;

N-[2-[N,N-bis-(2-methylpropyl)amino]thiazol-4-ylcarbonyl]-N′-(-tert-butoxycarbonyl-L-leucinyl)ydrazide;

N-[2-[N,N-bis-(2-methylpropyl)amino]thiazol-4-ylcarbonyl]-N′-[N-(L-leucinyl)]hydrazide;

N-[2-(1-naphthyl)thiazol-4-ylcarbonyl]-N′-[N-methyl-N-(3-pyridinylmethoxycarbonyl)-L-leucinyl]hydrazide;

N-[2-[N,N-bis-(2-methylpropyl)amino]thiazol-4-ylcarbonyl]-N′-[N-picolinoyl-L-leucinyl)hydrazide;

N-[2-[N,N-bis-(2-methylpropyl)amino]thiazol-4-ylcarbonyl]-N′-[N-(2-pyrazinecarbonyl)-L-leucinyl]hydrazide;

N-[N,N-bis-(2-methylpropyl)carbamoyl]-N′-[2-[N-(2-methylpropyl)-N-phenylamino]thiazol-4-ylcarbonyl]hydrazide;

N-(2-phenylthiazol-4-ylcarbonyl)-N′-[N-(4-pyridinylmethoxycarbonyl)-L-leucinyl]hydrazide;

N-[2-[2-(4-tert-butoxycarbonyl)benzyloxyphenyl]thiazol-4-ylcarbonyl]-N′-[N-(4-pyridinylmethoxycarbonyl)-L-leucinyl]hydrazide;

N-[2-[2-(4-carboxybenzyloxy)phenyl]thiazol-4-ylcarbonyl]-N′-[N-(4-pyridinylmethoxycarbonyl)-L-leucinyl]hydrazide;

N-[N-(4-tert-butoxycarbonylbenzyloxycarbonyl)-L-leucinyl]-N′-[2-[N-(2-methylpropyl)-N-phenylamino]thiazol-4-ylcarbonyl]hydrazide;

N-[2-[N,N-bis-(2-methylpropyl)amino]thiazol-4-ylcarbonyl]-N′-[N-(4-tert-butoxycarbonylbenzyloxycarbonyl)-L-leucinyl]hydrazide;

N-[N-(4-carboxybenzyloxycarbonyl)-L-leucinyl]-N′-[2-[N-(2-methylpropyl)-N-phenylamino]thiazol-4-ylcarbonyl]hydrazide;

N-(N-benzyloxycaronyl-L-leucinyl)-N′-[2-[2-(4-tert-butoxycarbonyl)benzyloxyphenyl]thiazol-4-ylcarbonyl]hydrazide;

N-(N-benzyloxycaronyl-L-leucinyl)-N′-[2-[2-(4-carboxybenzyloxy)phenyl]thiazol-4-ylcarbonyl]hydrazide;

N-[N-(6-methyl-3-pyridinylmethoxycarbonyl)-L-leucinyl]-N′-[2-(1-naphthyl)thiazol-4-ylcarbonyl]hydrazide;

N-(N-benzyloxycarbonyl-L-leucinyl)-N′-[2-(N-cyclopropyl-N-cyclopropylmethylamino)thiazol-4-ylcarbonyl]hydrazide;

N-[2-(N-cyclopropyl-N-cyclopropylmethylamino)thiazol-4-carbonyl]-N′-[N-(2-pyridinylmethoxycarbonyl)-L-leucinyl]hydrazide;

N-[2-(N-cyclopropyl-N-cyclopropylmethylamino)thiazol-4-ylcarbonyl]-N′-[N-(6-methyl-3-pyridinylmethoxycarbonyl)-L-leucinyl]hydrazide;

N-(N-tert-butoxycarbonyl-L-leucinyl)-N′-[2-(N-cyclopropyl-N-cyclopropylmethylamino)thiazol-4-ylcarbonyl]hydrazide;

N-[2-(N-cyclopropyl-N-cyclopropylmethylamino)thiazol-4-ylcarbonyl]-N′-[N-methyl-N-(2-pyridinylmethoxycarbonyl)-L-leucinyl]hydrazide;

N-[2-(2-benzyloxyphenyl)thiazol-4-ylcarbonyl]-N′-[N-(6-methyl-3-pyridinylmethoxycarbonyl)-L-leucinyl]hydrazide;

N-[2-(2-benzyloxyphenyl)thiazol-4-ylcarbonyl]-N′-[N-(2-methyl-3-pyridinylmethoxycarbonyl)-L-leucinyl]hydrazide;

N-[2-[N-methyl-N-(2-methylpropyl)amino]thiazol-4-ylcarbonyl]-N′-[N-(6-methyl-3-pyridinylmethoxycarbonyl)-L-leucinyl]hydrazide;

N-[2-[N-methyl-N-(2-methylpropyl)amino]thiazol-4-ylcarbonyl]-N′-[N-( )-L-leucinyl]hydrazide;

N-[2-(N-cyclopropyl-N-cyclopropylmethylamino)thiazol-4-ylcarbonyl]-N′-(N-picolinoyl-L-leucinyl)hydrazide;

N-[2-(N-cyclopropyl-N-cyclopropylmethylamino)thiazol-4-ylcarbonyl]-N′-[N-(2-methyl-3-pyridinylmethoxycarbonyl)-L-leucinyl]hydrazide;

N-[N-(3-tert-butoxycarbonylbenzyloxycarbonyl)-L-leucinyl]-N′-[2-(N-cyclopropyl-N-cyclopropylmethylamino)thiazol-4-ylcarbonyl]hydrazide;

N-[2-(1-naphthyl)thiazol-4-ylcarbonyl]-N′-[N-(8-quinolinoyl)-L-leucinyl]hydrazide;

N-[N-(2-methyl-3-pyridinylmethoxycarbonyl)-L-leucinyl]-N′-[2-(1-naphthyl)thiazol-4-ylcarbonyl]hydrazide;

N-[2-(1-naphthyl)thiazol-4-ylcarbonyl]-N′-(N-picolinoyl-L-leucinyl)hydrazide;

N-[N-(3-carboxybenzyloxycarbonyl)-L-leucinyl]-N′-[2-(N-cyclopropyl-N-cyclopropylmethylamino)thiazol-4-ylcarbonyl]hydrazide;

N-[2-(1-naphthyl)thiazol-4-ylcarbonyl]-N′-[N-(2-quinolinoyl)-L-leucinyl]hydrazide;

N-[2-(1-naphthyl)thiazol-4-ylcarbonyl]-N′-[N-(3-quinolinoyl)-L-leucinyl]hydrazide;

N-[2-(1-naphthyl)thiazol-4-ylcarbonyl]-N′-[N-(4-methylpiperidinecarbonyl)-L-leucinyl]hydrazide;

N-[2-(1-naphthyl)thiazol-4-ylcarbonyl]-N′-[N-(4-quinolinoyl)-L-leucinyl]hydrazide;

N-[2-(1-naphthyl)thiazol-4-ylcarbonyl]-N′-[N-(5-quinolinoyl)-L-leucinyl]hydrazide;

N-[2-(1-naphthyl)thiazol-4-ylcarbonyl]-N′-[N-(7-quinolinoyl)-L-leucinyl]hydrazide;

N-[2-(1-naphthyl)thiazol-4-ylcarbonyl]-N′-[N-(6-quinolinoyl)-L-leucinyl]hydrazide;

N-[N-(1-isoquinolinoyl)-L-leucinyl]-N′-[2-(1-naphthyl)thiazol-4-ylcarbonyl]hydrazide;

N-[N-(3-isoquinolinoyl)-L-leucinyl]-N′-[2-(1-naphthyl)thiazol-4-ylcarbonyl]hydrazide;

N-[N-(4-methylimidazol-5-ylcarbonyl)-L-leucinyl]-N′-[2-(1-naphthyl)thiazol-4-ylcarbonyl]hydrazide;

N-(N-benzyl-L-prolinyl-L-leucinyl)-N′-[2-(1-naphthyl)thiazol-4-ylcarbonyl]hydrazide;

N-[N-(1-benzyl-5-methylimidazol-4-ylcarbonyl)-L-leucinyl]-N′-[2-(1-naphthyl)thiazol-4-ylcarbonyl]hydrazide;

N-[N-(3-methylisonicotinoyl)-L-leucinyl]-N′-[2-(1-naphthyl)thiazol-4-ylcarbonyl]hydrazide;

N-[2-(N-cyclopropylamino)thiazol-4-ylcarbonyl]-N′-[N-(2-pyridinylmethoxycarbonyl)-L-leucinyl]hydrazide;

N-[4-methyl-2-(3-phenoxy)phenylpentanoyl]-N′-[2-(1-naphthyl)thiazol-4-ylcarbonyl]hydrazide;

N-[N-(2-benzoxazolyl)-L-leucinyl]-N′-[2-(1-naphthyl)thiazol-4-ylcarbonyl]hydrazide;

N-(N-benzyloxycarbonyl-L-leucinyl)-N′-[2-[N,N-bis-(2-methylpropyl)amino]oxazol-4-ylcarbonyl]hydrazide;

N-[2-[N-cyclopropyl-N-(2-methylpropyl)amino]thiazol-4-ylcarbonyl]-N′-[N-(2-pyridinylmethoxycarbonyl)-L-leucinyl]hydrazide;

N-[2-[N-cyclopropyl-N-(2-methylpropyl)amino]thiazol-4-ylcarbonyl]-N′-[N-methyl-N-(2-pyridinylmethoxycarbonyl)-L-leucinyl]hydrazide;

N-[2-(1-naphthyl)thiazol-4-ylcarbonyl]-N′-[N-(1-piperazinecarbonyl)-L-leucinyl]hydrazide;

N-[4-methyl-2-(4-phenoxy)phenylpentanoyl]-N′-[2-(1-naphthyl)thiazol-4-ylcarbonyl]hydrazide;

N-[2-[N-bis-(cyclopropylmethyl)amino]thiazol-4-ylcarbonyl]-N′-[N-(2-pyridinylmethoxycarbonyl)-L-leucinyl]hydrazide;

N-[2-(N-cyclopropyl-N-cyclopropylmethylamino)thiazol-4-ylcarbonyl]-N′-[N-(2-quinolinoyl)-L-leucinyl]hydrazide;

N-[N-(8-quinolinoyl)-L-leucinyl]-N′-[2-(8-quinolinyl)thiazol-4-ylcarbonyl]hydrazide;

N-(N-benzyloxycarbonyl-L-leucinyl]-N′-[2-(1-naphthyl)thiazol-4-ylcarbonyl]hydrazide;

N-[2-(N-cyclopropyl-N-cyclopropylmethylamino)thiazol-4-ylcarbonyl]-N′-[N-(3-quinolinoyl)-L-leucinyl]hydrazide;

N-[2-(N-cyclopropyl-N-cyclopropylmethylamino)thiazol-4-ylcarbonyl]-N′-[N-(3-isoquinolinoyl)-L-leucinyl]hydrazide;

N-[2-(N-cyclopropyl-N-cyclopropylmethylamino)thiazol-4-ylcarbonyl]-N′-[N-(6-quinolinoyl)-L-leucinyl]hydrazide;

N-[2-[N-bis-(cyclopropylmethyl)amino]thiazol-4-ylcarbonyl]-N′-[N-(2-methyl-3-pyridinylmethoxycarbonyl)-L-leucinyl]hydrazide;

N-(N-benzyloxycarbonyl-L-b-tert-butylalanyl)-N′-[2-(1-naphthyl)thiazol-4-ylcarbonyl]hydrazide;

N-(N-benzyloxycarbonyl-L-b-cyclopropylalanyl)-N′-[2-(1-naphthyl)thiazol-4-ylcarbonyl]hydrazide;

N-[2-(1-naphthyl)thiazol-4-ylcarbonyl]-N′-[N-[3-(2-pyridyl)phenylacetyl]-L-leucinyl]hydrazide;

N-[2-[N-bis-(cyclopropylmethyl)amino]thiazol-4-ylcarbonyl]-N′-(N-picolinyl-L-leucinyl)hydrazide;

N-(N-benzyloxycarbonyl-L-leucinyl)-N′-[2-N-bis-(cyclopropylmethyl)amino]thiazol4-ylcarbonyl]hydrazide;

N-[2-(N-cyclopropyl-N-cyclopropylmethylamino)thiazol-4-ylcarbonyl]-N′-[N-(6-methylnicotinoyl)-L-leucinyl]hydrazide;

N-[2-(N-cyclopropyl-N-cyclopropylmethylamino)thiazol-4-ylcarbonyl]-N′-[N-(2-methylnicotinoyl)-L-leucinyl]hydrazide;

N-[2-(N-cyclopropyl-N-cyclopropylmethylamino)thiazol-4-ylcarbonyl]-N′-[N-(3-methylisonicotinoyl)-L-leucinyl]hydrazide;

N-[2-(N-cyclopropyl-N-cyclopropylmethylamino)thiazol-4-ylcarbonyl]-N′-[N-(8-quinolinoyl)-L-leucinyl]hydrazide;

N-[2-[N-bis-(cyclopropylmethyl)amino]thiazol-4-ylcarbonyl]-N′-[N-(8-quinolinoyl)-L-leucinyl]hydrazide;

N-[2-[N-bis-(cyclopropylmethyl)amino)thiazol-4-ylcarbonyl]-N′-[N-(3-isoquinolinoyl)-L-leucinyl]hydrazide;

N-[2-[4-(2,2-dimethylaminoethoxy)-1-naphthyl]thiazol-4-ylcarbonyl]-N′-[N-(8-quinolinoyl)-L-leucinyl]hydrazide;

N-[2-(N-cyclopropyl-N-cyclopropylmethylamino)thiazol-4-ylcarbonyl]-N′-[N-(7-quinolinoyl)-L-leucinyl]hydrazide;

N-[2-[N-bis-(cyclopropylmethyl)amino]thiazol-4-ylcarbonyl]-N′-[N-(6-methylnicotinoyl)-L-leucinyl]hydrazide;

N-[2-[N-bis-(cyclopropylmethyl)amino]thiazol-4-ylcarbonyl]-N′-(N-methyl-L-prolinyl-L-leucinyl)hydrazide;

N-(N-benzyloxycarbonyl-L-norvalinyl)-N′-[2-(1-naphthyl)thiazol-4-ylcarbonyl]hydrazide;

N-(N-benzyloxycarbonyl-L-isoleucinyl)-N′-[2-(1-naphthyl)thiazol-4-ylcarbonyl]hydrazide;

N-[N-(4-dimethylaminomethylbenzoyl)-L-leucinyl]-N′-[2-(1-naphthyl)thiazol-4-ylcarbonyl]hydrazide;

N-(N-benzyloxycarbonyl-L-norleucinyl)-N′-[2-(1-naphthyl)thiazol-4-ylcarbonyl]hydrazide;

N-[N-(4-dimethylaminomethylbenzyloxycarbonyl)-L-leucinyl]-N′-[2-(1-naphthyl)thiazol-4-ylcarbonyl]hydrazide;

N-(N-benzyloxycarbonyl-L-norvalinyl)-N′-[2-(2-benzyloxyphenyl)thiazol-4-ylcarbonyl]hydrazide;

N-[2-(N-cyclopropyl-N-cyclopropylmethylamino)thiazol-4-ylcarbonyl]-N′-[N-(4-methylimidazol-5-ylcarbonyl)-L-leucinyl]hydrazide;

N-[N-[4-(4-morpholinomethyl)benzoyl]-L-leucinyl]-N′-[2-(1-naphthyl)thiazol-4-ylcarbonyl]hydrazide;

N-[N-(2-methylnicotinoyl)-L-leucinyl]-N′-[2-(1-naphthyl)thiazol-4-ylcarbonyl]hydrazide;

N-[N-(6-methylnicotinoyl)-L-leucinyl]-N′-[2-(1-naphthyl)thiazol-4-ylcarbonyl]hydrazide;

N-(N-b-tert-butoxycarbonyl-L-tert-butylalanyl)-N′-2-(N-cyclopropyl-N-cyclopropylmethylamino)thiazol-4-ylcarbonyl]hydrazide;

N-[2-(N-cyclopropyl-N-cyclopropylmethylamino)thiazol-4-ylcarbonyl]-N′-[N-(8-quinolinoyl)-L-b-tert-butylalanyl]hydrazide;

N-[N-(4-methylimidazol-5-ylcarbonyl)-L-allylglycinyl]-N′-[2-(1-naphthyl)thiazol-4-ylcarbonyl]hydrazide;

N-[N-(4-methylimidazol-5-ylcarbonyl)-L-b-tert-butylalanyl]-N′-[2-(1-naphthyl)thiazol-4-ylcarbonyl]hydrazide;

N-[2-(N-cyclopropyl-N-cyclopropylmethylamino)thiazol-4ylcarbonyl]-N′-[N-(4-methylimidazol-5-ylcarbonyl)-L-b-tert-butylalanyl]hydrazide;

N-[2-(1-naphthyl)thiazol-4-ylcarbonyl]-N′-(N-picolinoyl-L-b-tert-butylalanyl)hydrazide;

N-[2-(1-naphthyl)thiazol-4-ylcarbonyl]-N′-[N-(8-quinolinoyl)-L-b-tert-butylalanyl]hydrazide;

N-[2-(1-naphthyl)thiazol-4-ylcarbonyl]-N′-(N-picolinyl-L-allylglycinyl)hydrazide;

N-[2-(1-naphthyl)thiazol-4-ylcarbonyl]-N′-(N-picolinoyl-L-b-cyclopropylalanyl)hydrazide;

N-[N-(6-methylnicotinoyl)-L-b-cyclopropylalanyl]-N′-[2-(1-naphthyl)thiazol-4-ylcarbonyl]hydrazide;

N-[N-(4-methylimidazol-5-ylcarbonyl)-L-b-cyclopropylalanyl]-N′-[2-(1-naphthyl)thiazol-4-ylcarbonyl]hydrazide;

N-[2-(1-naphthyl)thiazol-4-ylcarbonyl]-N′-[N-(8-quinolinoyl)-L-b-cyclopropylalanyl]hydrazide;

N-[N-(6-methylnicotinoyl)-L-b-tert-butylalanyl]-N′-[2-(1-naphthyl)thiazol-4-ylcarbonyl]hydrazide;

N-[2-(N-cyclopropyl-N-cyclopropylmethylamino)thiazol-4-ylcarbonyl]-N′-(N-picolinoyl-L-b-tert-butylalanyl)hydrazide;

N-[2-(N-cyclopropyl-N-cyclopropylmethylamino)thiazol-4-ylcarbonyl]-N′-[N-(3-isoquinolinoyl)-L-b-tert-butylalanyl]hydrazide;

N-(N-tert-butoxycarbonyl-L-b-cyclopropylalanyl)-N′-[2-(N-cyclopropyl-N-cyclopropylmethylamino)thiazol-4-ylcarbonyl]hydrazide;

N-[2-(N-cyclopropylmethyl-N-propylamino)thiazol-4-ylcarbonyl]-N′-[N-(6-methyl-3-pyridinylmethoxycarbonyl)-L-leucinyl]hydrazide;

N-[N-(6-methylnicotinoyl)-L-allylglycinyl]-N′-[2-(1-naphthyl)thiazol-4-ylcarbonyl]hydrazide;

N-[2-(1-naphthyl)thiazol-4-ylcarbonyl]-N′-[N-(8-quinolinoyl)-L-allylglycinyl]hydrazide;

N-[2-(1-naphthyl)thiazol-4-ylcarbonyl]-N′-[N-(2-quinolinoyl)-L-b-cyclopropylalanyl]hydrazide;

N-[N-(3-isoquinolinoyl)-L-b-cyclopropylalanyl]-N′[-2-(1-naphthyl)thiazol-4-ylcarbonyl]hydrazide;

N-[N-(1-isoquinolinoyl)-L-b-cyclopropylalanyl]-N′-[2-(1-naphthyl)thiazol-4-ylcarbonyl]hydrazide;

N-[2-(1-naphthyl)thiazol-4-ylcarbonyl]-N′-[N-(7-quinolinoyl)-L-b-cyclopropylalanyl]hydrazide;

N-[2-(N-cyclopropyl-N-cyclopropylmethylamino)thiazol-4-ylcarbonyl]-N′-[N-(8-quinolinoyl)-L-b-cyclopropylalanyl]hydrazide;

N-[2-(N-cyclopropyl-N-cyclopropylmethylamino)thiazol-4-ylcarbonyl]-N′-[N-(4-methylimidazol-5-ylcarbonyl)-L-b-cyclopropylalanyl]hydrazide;

N-[2-(N-cyclopropyl-N-cyclopropylmethylamino)thiazol-4-ylcarbonyl]-N′-[N-(3-isoquinolinoyl)-L-b-cyclopropylalanyl]hydrazide;

N-[2-(N-cyclopropyl-N-cyclopropylmethylamino)thiazol-4-ylcarbonyl]-N′-[N-(6-methylnicotinoyl)-L-b-cyclopropylalanyl]hydrazide;

N-[N-(4-methylimidazol-5-ylcarbonyl)-L-norleucinyl]-N′-[2-(1-naphthyl)thiazol-4-ylcarbonyl]hydrazide;

N-[2-(1-naphthyl)thiazol-4-ylcarbonyl]-N′-(N-picolinoyl-L-norleucinyl)hydrazide;

N-[2-(1-naphthyl)thiazol-4-ylcarbonyl]-N′-[N-(8-quinolinoyl)-L-norleucinyl]hydrazide;

N-[2-(N-cyclopropyl-N-cyclopropylmethylamino)thiazol-4-ylcarbonyl]-N′-[N-(2-quinolinoyl)-L-b-cyclopropylalanyl]hydrazide;

N-[2-(N-cyclopropyl-N-cyclopropylmethylamino)thiazol-4-ylcarbonyl]-N′[N-(1-isoquinolinoyl)-L-b-cyclopropylalanyl]hydrazide;

N-[2-[N-cyclopropyl-N-(2-methylpropyl)amino]thiazol-4-ylcarbonyl]-N′-[N-(6-methyl-3-pyridinylmethoxycarbonyl)-L-leucinyl]hydrazide;

N-(N-tert-butoxycarbonyl-L-leucinyl)-N′-[2-[N-cyclopropyl-N-(2-methylpropyl)amino]thiazol-4-ylcarbonyl]hydrazide;

N-[2-(1-naphthyl)thiazol-4-ylcarbonyl]-N′-[N-(7-quinolinoyl)-L-b-tert-butylalanyl]hydrazide;

N-[2-(1-naphthyl)thiazol-4-ylcarbonyl]-N′-[N-(2-quinolinoyl)-L-b-tert-butylalanyl]hydrazide;

N-[N-(1-isoquinolinoyl)-L-b-tert-butylalanyl]-N′-[2-(1-naphthyl)thiazol-4-ylcarbonyl]hydrazide;

N-[N-(3-isoquinolinoyl)-L-b-tert-butylalanyl]-N′-[2-(1-naphthyl)thiazol-4-ylcarbonyl]hydrazide;

N-[N-(6-methylnicotinoyl)-L-norleucinyl]-N′-[2-(1-naphthyl)thiazol-4-ylcarbonyl]hydrazide;

N-[2-(1-naphthyl)thiazol-4-ylcarbonyl]-N′-[N-(7-quinolinoyl)-L-norleucinyl]hydrazide;

N-[2-(1-naphthyl)thiazol-4-ylcarbonyl]-N′-[N-(2-quinolinoyl)-L-norleucinyl]hydrazide;

N-[N-(1-isoquinolinoyl)-L-norleucinyl]-N′-[2-(1-naphthyl)thiazol-4-ylcarbonyl]hydrazide;

N-[N-(3-isoquinolinoyl)-L-norleucinyl]-N′-[2-(1-naphthyl)thiazol-4-ylcarbonyl]hydrazide;

N-[2-(N-cyclopropyl-N-cyclopropylmethylamino)thiazol-4-ylcarbonyl]-N′-[N-(5-hydroxymethylimidazol-4-ylcarbonyl)-L-b-cyclopropylalanyl]hydrazide;

N-[2-[N-cyclopropyl-N-(2-methylpropyl)amino]thiazol-4-ylcarbonyl]-N′-[N-(8-quinolinoyl)-L-b-cyclopropylalanyl]hydrazide;

N-[2-(N-cyclopropyl-N-cyclopropylmethylamino)thiazol-4-ylcarbonyl]-N′-(N-(6-methylnicotinoyl)-L-b-tert-butylalanyl]hydrazide;

N-[2-[N-cyclopropyl-N-(2-methylpropyl)amino]thiazol-4-ylcarbonyl]-N′-[N-4methylimidazol-5-ylcarbonyl)-L-b-cyclopropylalanyl]hydrazide;

N-[2-[N-cyclopropyl-N-(2-methylpropyl)amino]thiazol-4-ylcarbonyl]-N′-[N-(2-quinolinoyl)-L-b-cyclopropylalanyl]hydrazide;

N-[2-[N-cyclopropyl-N-(2-methylpropyl)amino]thiazol-4-ylcabonyl]-N′-[N-(6-methylnicotinoyl)-L-b-cyclopropylalanyl]hydrazide;

N-[2-(1-naphthyl)thiazol-4-ylcarbonyl]-N′-[N-(8-quinolinoyl)glycinyl]hydrazide;

N-[2-(1-naphthyl)thiazol-4-ylcarbonyl]-N′-[N-(8-quinolinoyl)-L-norvalinyl]hydrazide;

N-[2-(1-naphthyl)thiazol-4-ylcarbonyl]-N′-[N-(2-quinolinoyl)-L-norvalinyl]hydrazide;

N-[2-(1-naphthyl)thiazol 4-ylcarbonyl]-N′-(N-picolinoyl-L-norvalinyl]hydrazide,

N-[2-(1-naphthyl)thiazol-4-ylcarbonyl]-N′-[N-(6-methylnicotinoyl)-L-norvalinyl]hydrazide;

N-[2-(1-naphthyl)thiazol-4-ylcarbonyl]-N′-[N-(4-methylimidazol-5-ylcarbonyl)-L-norvalinyl]hydrazide;

N-[2-(1-naphthyl)thiazol-4-ylcarbonyl]-N′-[N-(1-isoquinolinoyl)-L-norvalinyl]hydrazide;

N-[2-(1-naphthyl)thiazol-4-ylcarbonyl]-N′-[N-(3-isoquinolinoyl)-L-norvalinyl]hydrazide; (1S, 1′S)-N,N′-bis-[4-[1-(N-benzyloxycarbonylamino)-3-methylbutyl]thiazol-2-ylcarbonyl]hydrazide;

N-[2-[N-cyclopropyl-N-(2-methylpropyl)amino]thiazol-4-ylcarbonyl]-N′-[N-(6-methylnicotinoyl)-L-b-tert-butylalanyl]hydrazide;

N-[2-[N-cyclopropyl-N-(2-methylpropyl)amino]thiazol-4-ylcarbonyl]-N′-[N-(4-methylimidazol-5-ylcarbonyl)-L-b-tert-butylalanyl]hydrazide;

N-[2-(N-cyclopropyl-N-cyclopropylmethylamino)thiazol-4-ylcarbonyl]-N′-[N-(1-isoquinolinoyl)-L-b-tert-butylalanyl]hydrazide;

N-[N-(5-butylpicolinoyl)-L-b-tert-butylalanyl]-N′-[2-(N-cyclopropyl-N-cyclopropylmethylamino)thiazol-4-ylcarbonyl]hydrazide;

N-[2-(N-cyclopropyl-N-cyclopropylmethylamino)thiazol-4-ylcarbonyl]-N′-[N-(6-methylpicolinoyl)-L-b-tert-butylalanyl]hydrazide;

N-[2-(N-cyclopropyl-N-cyclopropylmethylamino)thiazol-4-ylcarbonyl]-N′-[N-(4-fluorobenzoyl)-L-leucinyl]hydrazide;

N-[N-(4fluorobenzoyl)-L-leucinyl-N′-[2-(1-naphthyl)thiazol-4-ylcarbonyl]hydrazide;

N-[2-(1-naphthyl)thiazol-4-ylcarbonyl]-N′-[N-(2-pyridinylmethoxycarbonyl)-L-b-tert-butylalanyl]hydrazide;

N-[N-(2-methyl-3-pyridinylmethoxycarbonyl)-L-b-tert-butylalanyl]-N′-[2-(1-naphthyl)thiazol-4-ylcarbonyl]hydrazide;

N-[2-(1-naphthyl)thiazol-4-ylcarbonyl]-N′-[N-(2-pyridinylmethoxycarbonyl)-L-b-cyclopropylalanyl]hydrazide;

N-[N-(2-methyl-3-pyridinylmethoxycarbonyl)-L-b-cyclopropylalanyl]-N′-[2-(1-naphthyl)thiazol-4-ylcarbonyl]hydrazide;

N-[N-(6-methyl-3-pyridinylmethoxycarbonyl)-L-b-cyclopropylalanyl]-N′-[2-(1-naphthyl)thiazol-4-ylcarbonyl]hydrazide;

N-[N-(6-methyl-3-pyridinylmethoxycarbonyl)-L-b-tert-butylalanyl]-N′-[2-(1-naphthyl)thiazol-4-ylcarbonyl]hydrazide;

N,N′-bis-[2-(1-naphthyl)thiazol-4-ylcarbonyl]hydrazide;

N-[2-(N-cyclopropyl-N-cyclopropylmethylamino)thiazol-4-ylcarbonyl]-N′-[N-[2-(1,8-naphthyridinoyl)]-L-b-cyclopropylalanyl]hydrazide;

N-[2-[N-cyclopropyl-N-(2-methylpropyl)amino]thiazol-4-ylcarbonyl]-N′-[N-(3,4-difluorobenzoyl)-L-b-cyclopropylalanyl]hydrazide;

N-[2-[N-cyclopropyl-N-(2-methylpropyl)amino]thiazol-4-ylcarbonyl]-N′-[N-(4-flluorobenzoyl)-L-leucinyl]hydrazide;

N-[N-(5-butylpicolinoyl)-L-leucinyl]-N′-[2-(N-cyclopropyl-N-cyclopropylmethylamino)thiazol-4-ylcarbonyl]hydrazide;

N-[2-[N-cyclopropyl-N-(2-methylpropyl)amino]thiazol-4-ylcarbonyl]-N′-[N-(3,4-dimethoxybenzoyl)-L-leucinyl]hydrazide;

N-[2-[N-cyclopropyl-N-(2-methylpropyl)amino]thiazol-4-ylcarbonyl]-N′-[N-(3,4-difluorobenzoyl)-L-b-tert-butylalanyl]hydrazide;

N-[2-[N-cyclopropyl-N-(2-methylpropyl)amino]thiazol-4-ylcarbonyl]-N′-[N-(3,4-dimethoxybenzoyl)-L-b-tert-butylalanyl]hydrazide;

N-[N-(5-butylpicolinoyl)-L-b-tert-butylalanyl]-N′-[2-(N-cyclopropyl-N-cyclopropylmethylamino)thiazol-4-ylcarbonyl]hydrazide; and

N-[2-[N-cyclopropyl-N-(2-methylpropyl)amino]thiazol-4-ylcarbonyl]-N′-[N-(6-methylpicolinoyl)-L-b-tert-butylalanyl]hydrazide.

Most particularly preferred compounds of the present invention include:

N-[2-[N-cyclopropylmethyl-N-(2-methylpropyl)amino]thiazol-4-ylcarbonyl]-N′-[N-(4-pyridinylmethoxycarbonyl)-L-leucinyl]hydrazide;

N-[2-(4-methyl-1-naphthyl)thiazol-4-ylcarbonyl]-N′-[N-(4-pyridinylmethoxycarbonyl)-L-leucinyl]hydrazide;

N-[2-[N-methyl-N-(2-methylpropyl)amino]thiazol-4-ylcarbonyl]-N′-[N-methyl-N-(4-pyridinylmethoxycarbonyl)-L-leucinyl]hydrazide;

N-[2-(1-naphthyl)thiazol-4-ylcarbonyl]-N′-[N-(3-pyridinylmethoxycarbonyl)-L-leucinyl]hydrazide;

N-[2-(1-naphthyl)thiazol-4-ylcarbonyl]-N′-[N-(2-pyridinylmethoxycarbonyl)-L-leucinyl]hydrazide;

N-[2-(5-acenaphthyl)thiazol-4-ylcarbonyl]-N′-[N-(4-pyridinylmethoxycarbonyl)-L-leucinyl]hydrazide;

N-[2-[N-cyclopropylmethyl-N-(2-methylpropyl)amino]thiazol-4-ylcarbonyl]-N′-[N-methyl-N-(4-pyridinylmethoxycarbonyl)-L-leucinyl]hydrazide;

N-[2-(N-cyclopropyl-N-cyclopropylmethylamino)thiazol-4-ylcarbonyl]-N′-[N-(4-pyridinylmethoxycarbonyl)-L-leucinyl]hydrazide;

N-[2-[N-cyclopropylmethyl-N-(2-methylpropyl)amino]thiazol-4-ylcarbonyl]-N′-[N-(3-pyridinylmethoxycarbonyl)-L-leucinyl]hydrazide;

N-[2-[N-cyclopropylmethyl-N-(2-methylpropyl)amino]thiazol-4-ylcarbonyl]-N′-[N-(2-pyridinylmethoxycarbonyl)-L-leucinyl]hydrazide;

N-[2-[N-cyclopropylmethyl-N-(2-methylpropyl)amino]thiazol-4-ylcarbonyl]-N′-[N-methyl-N-(3-pyridinylmethoxycarbonyl)-L-leucinyl]hydrazide;

N-[2-(N-cyclopropyl-N-cyclopropylmethylamino)thiazol-4-ylcarbonyl]-N′-[N-(3-pyridinylmethoxycarbonyl)-L-leucinyl]hydrazide;

N-[2-(N-cyclopropyl-N-cyclopropylmethylamino)thiazol-4-ylcarbonyl]-N′-[N-methyl-N-(4-pyridinylmethoxycarbonyl)-L-leucinyl]hydrazide;

N-[2-[N,N-bis-(2-methylpropyl)amino]thiazol-4-ylcarbonyl]-N′-[N-(2-pyridinylmethoxycarbonyl)-L-leucinyl]hydrazide;

N-[N-(4-pyridinylmethoxycarbonyl)-L-leucinyl]-N′-[2-[1-(1,2,3,4-tetrahydroquinolino)]thiazol-4-ylcarbonyl]hydrazide;

N-[2-[N,N-bis-(2-methylpropyl)amino]thiazol-4-ylcarbonyl]-N′-[N-methyl-N-(3-pyridinylmethoxycarbonyl)-L-leucinyl]hydrazide;

N-[2-(N-cyclopropyl-N-cyclopropylmethylamino)thiazol-4-ylcarbonyl]-N′-[N-methyl-N-(3-pyridinylmethoxycarbonyl)-L-leucinyl]hydrazide;

N-[2-(N-cyclopropylmethyl-N-propylamino)thiazol-4-ylcarbonyl]-N′-[N-(3-pyridinylmethoxycarbonyl)-L-leucinyl]hydrazide;

N-[2-(2-benzyloxy-1-naphthyl)thiazol-4-ylcarbonyl]-N′-[N-(4-pyridinylmethoxycarbonyl)-L-leucinyl]hydrazide;

N-[2-[N,N-bis-(2-methylpropyl)amino]thiazol-4-ylcarbonyl]-N′-[N-methyl-N-(2-pyridinylmethoxycarbonyl)-L-leucinyl]hydrazide;

N-[2-(9-phenanthrenyl)thiazol-4-ylcarbonyl]-N′-[N-(4-pyridinylmethoxycarbonyl)-L-leucinyl]hydrazide;

N-[2-[N,N-bis-(2-methylpropyl)amino]thiazol-4-ylcarbonyl]-N′-(-tert-butoxycarbonyl-L-leucinyl)ydrazide;

N-[2-[N,N-bis-(2-methylpropyl)amino]thiazol-4-ylcarbonyl]-N′-(N-picolinoyl-L-leucinyl)hydrazide;

N-[2-[N,N-bis-(2-methylpropyl)amino]thiazol-4-ylcarbonyl]-N′-[N-(2-pyrazinecarbonyl)-L-leucinyl]hydrazide;

N-[2-[2-(4-tert-butoxycarbonyl)benzyloxyphenyl]thiazol-4-ylcarbonyl]-N′-[N-(4-pyridinylmethoxycarbonyl)-L-leucinyl]hydrazide;

N-[2-[2-(4-carboxybenzyloxy)phenyl]thiazol-4-ylcarbonyl]-N′-[N-(4-pyridinylmethoxycarbonyl)-L-leucinyl]hydrazide;

N-[N-(4-tert-butoxycarbonylbenzyloxycarbonyl)-L-leucinyl]-N′-[2-[N-(2-methylpropyl)-N-phenylamino]thiazol-4-ylcarbonyl]hydrazide;

N-[2-[N,N-bis-(2-methylpropyl)amino]thiazol-4-ylcarbonyl]-N′-[N-(4-tert-butoxycarbonylbenzyloxycarbonyl)-L-leucinyl]hydrazide;

N-[N-(4-carboxybenzyloxycarbonyl)-L-leucinyl]-N′-[2-[N-(2-methylpropyl)-N-phenylamino]thiazol-4-ylcarbonyl]hydrazide;

N-[N-(6-methyl-3-pyridinylmethoxycarbonyl)-L-leucinyl]-N′-[2-(1-naphthyl)thiazol-4-ylcarbonyl]hydrazide;

N-(N-benzyloxycarbonyl-L-leucinyl)-N′-[2-(N-cyclopropyl-N-cyclopropylmethylamino)thiazol-4-ylcarbonyl]hydrazide;

N-[2-(N-cyclopropyl-N-cyclopropylmethylamino)thiazol-4-ylcarbonyl]-N′-[N-(2-pyridinylmethoxycarbonyl)-L-leucinyl]hydrazide;

N-[2-(N-cyclopropyl-N-cyclopropylmethylamino)thiazol-4-ylcarbonyl]-N′-[N-(6-methyl-3-pyridinylmethoxycarbonyl)-L-leucinyl]hydrazide;

N-(N-tert-butoxycarbonyl-L-leucinyl)-N′-[2-(N-cyclopropyl-N-cyclopropylmethylamino)thiazol-4-ylcarbonyl]hydrazide;

N-[2-(N-cyclopropyl-N-cyclopropylmethylamino)thiazol-4-ylcarbonyl]-N′-[N-methyl-N-(2-pyridinylmethoxycarbonyl)-L-leucinyl]hydrazide;

N-[2-(2-benzyloxyphenyl)thiazol-4-ylcarbonyl]-N′-[N-(6-methyl-3-pyridinylmethoxycarbonyl)-L-leucinyl]hydrazide;

N-[2-(2-benzyloxyphenyl)thiazol-4-ylcarbonyl]-N′-[N-(2-methyl-3-pyridinylmethoxycarbonyl)-L-leucinyl]hydrazide;

N-[2-[N-methyl-N-(2-methylpropyl)amino]thiazol-4-ylcarbonyl]-N′-[N-(6-methyl-3-pyridinylmethoxycarbonyl)-L-leucinyl]hydrazide;

N-[2-[N-methyl-N-(2-methylpropyl)amino]thiazol-4-ylcarbonyl]-N′-[N-( )-L-leucinyl]hydrazide;

N-[2-(N-cyclopropyl-N-cyclopropylmethylamino)thiazol-4-ylcarbonyl]-N′-(N-picolinoyl-L-leucinyl)hydrazide;

N-[2-(N-cyclopropyl-N-cyclopropylmethylamino)thiazol-4-ylcarbonyl]-N′-[N-(2-methyl-3-pyridinylmethoxycarbonyl)-L-leucinyl]hydrazide;

N-[N-(3-tert-butoxycarbonylbenzyloxycarbonyl)-L-leucinyl]-N′-[2-(N-cyclopropyl-N-cyclopropylmethylamino)thiazol-4-ylcarbonyl]hydrazide;

N-[2-(1-naphthyl)thiazol-4-ylcarbonyl]-N′-[N-(8-quinolinoyl-L-leucinyl]hydrazide;

N-[N-(2-methyl-3-pyridinylmethoxycarbonyl)-L-leucinyl]-N′-[2-(1-naphthyl)thiazol-4-ylcarbonyl]hydrazide;

N-[2-(1-naphthyl)thiazol-4-ylcarbonyl]-N′-(N-picolinoyl-L-leucinyl)hydrazide;

N-[N-(3-carboxybenzyloxycarbonyl)-L-leucinyl]-N′-[2-(N-cyclopropyl-N-cyclopropylmethylamino)thiazol-4-ylcarbonyl]hydrazide;

N-[2-(1-naphthyl)thiazol-4-ylcarbonyl]-N′-[N-(2-quinolinoyl)-L-leucinyl]hydrazide;

N-[2-(1-naphthyl)thiazol-4-ylcarbonyl]-N′-[N-(3-quinolinoyl)-L-leucinyl]hydrazide;

N-[2-(1-naphthyl)thiazol-4-ylcarbonyl]-N′-[N-(4-methylpiperidinecarbonyl)-L-leucinyl]hydrazide;

N-[2-(1-naphthyl)thiazol-4-ylcarbonyl]-N′-[N-(4-quinolinoyl)-L-leucinyl]hydrazide;

N-[2-(1-naphthyl)thiazol-4-ylcarbonyl]-N′-[N-(5-quinolinoyl)-L-leucinyl]hydrazide;

N-[2-(1-naphthylithiazol-4-ylcarbonyl]-N′-[N-(7-quinolinoyl)-L-leucinyl]hydrazide;

N-[2-(1-naphthyl)thiazol-4-ylcarbonyl]-N′-[N-(6-quinolinoyl)-L-leucinyl]hydrazide;

N-[N-(1-isoquinolinoyl)-L-leucinyl]-N′-[2-(1-naphthyl)thiazol-4-ylcarbonyl]hydrazide;

N-[N-(3-isoquinolinoyl)-L-leucinyl]-N′-[2-(1-naphthyl)thiazol-4-ylcarbonyl]hydrazide;

N-[N-(4-methylimidazol-5-ylcarbonyl)-L-leucinyl]-N′-[2-(1-naphthyl)thiazol-4-ylcarbonyl]hydrazide;

N-[N-(1-benzyl-5-methylimidazol-4-ylcarbonyl)-L-leucinyl]-N′-[2-(1-naphthyl)thiazol-4-ylcarbonyl]hydrazide;

N-[N-(3-methylisonicotinoyl)-L-leucinyl]-N′-[2-(1-naphthyl)thiazol-4-ylcarbonyl]hydrazide;

N-(N-benzyloxycarbonyl-L-leucinyl)-N′-[2-[N,N-bis-(2-methylpropyl)amino]oxazol-4-ylcarbonyl]hydrazide;

N-[2-[N-cyclopropyl-N-(2-methylpropyl)amino]thiazol-4-ylcarbonyl]-N′-[N-(2-pyridinylmethoxycarbonyl)-L-leucinyl]hydrazide;

N-[2-[N-cyclopropyl-N-(2-methylpropyl)amino]thiazol-4-ylcarbonyl]-N′-[N-methyl-N-(2-pyridinylmethoxycarbonyl)-L-leucinyl]hydrazide;

N-[2-(1-naphthyl)thiazol-4-ylcarbonyl]-N′-[N-(1-piperazinecarbonyl)-L-leucinyl]hydrazide;

N-[2-[N-bis-(cyclopropylmethyl)amino]thiazol-4-ylcarbonyl]-N′-[N-(2-pyridinylmethoxycarbonyl)-L-leucinyl]hydrazide;

N-[2-(N-cyclopropyl-N-cyclopropylmethylamino)thiazol-4-ylcarbonyl]-N′-[N-(2-quinolinoyl)-L-leucinyl]hydrazide;

N-[N(8-quinolinoyl)-L-leucinyl]-N′-[2-(8-quinolinyl)thiazol-4-ylcarbonyl]hydrazide;

N-(N-benzyloxycarbonyl-L-leucinyl)-N′-[2-(1-naphthyl)thiazol-4-ylcarbonyl]hydrazide;

N-[2-(N-cyclopropyl-N-cyclopropylmethylamino)thiazol-4-ylcarbonyl]-N′-[N-(3-quinolinoyl)-L-leucinyl]hydrazide;

N-[2-(N-cyclopropyl-N-cyclopropylmethylamino)thiazol-4-ylcarbonyl]-N′-[N-(3-isoquinolinoyl)-L-leucinyl]hydrazide;

N-[2-(N-cyclopropyl-N-cyclopropylmethylamino)thiazol-4-carbonyl]-N′-[N-(6-quinolinoyl)-L-leucinyl]hydrazide;

N-[2-[N-bis-(cyclopropylmethyl)amino]thiazol-4-ylcarbonyl]-N′-[N-(2-methyl-3-pyridinylmethoxycarbonyl)-L-leucinyl]hydrazide;

N-(N-benzyloxycarbonyl-L-b-tert-butylalanyl)-N′-[2-(1-naphthyl)thiazol-4-ylcarbonyl]hydrazide;

N-(N-benzyloxycarbonyl-L-b-cyclopropylalanyl)-N′-[2-(1-naphthyl)thiazol-4-ylcarbonyl]hydrazide;

N-[2-(1-naphthyl)thiazol-4-ylcarbonyl]-N′-[N-[3-(2-pyridyl)phenylacetyl]-L-leucinyl]hydrazide;

N-[2-[N-bis-(cyclopropylmethyl)amino]thiazol-4-ylcarbonyl]-N′-(N-picolinyl-L-leucinyl)hydrazide;

N-(N-benzyloxycarbonyl-L-leucinyl)-N′-[2-[N-bis-(cyclopropylmethyl)amino]thiazol-4-ylcarbonyl]hydrazide;

N-[2-(N-cyclopropyl-N-cyclopropylmethylamino)thiazol-4-ylcarbonyl]-N′-[N-(6-methylnicotinoyl)-L-leucinyl]hydrazide;

N-[2-(N-cyclopropyl-N-cyclopropylmethylamino)thiazol-4-ylcarbonyl]-N′-[N-(3-methylisonicotinoyl)-L-leucinyl]hydrazide;

N-[2-(N-cyclopropyl-N-cyclopropylmethylamino)thiazol-4-ylcarbonyl]-N′-[N-(8-quinolinoyl)-L-leucinyl]hydrazide;

N-[2-[N-bis-(cyclopropylmethyl)amino]thiazol-4-ylcarbonyl]-N′-[N-(8-quinolinoyl)-L-leucinyl]hydrazide;

N-[2-[N-bis-(cyclopropylmethylamino]thiazol-4-ylcarbonyl]-N′-[N-(3-isoquinolinoyl)-L-leucinyl]hydrazide;

N-[2-[4-(2,2-dimethylaminoethoxy)-1-naphthyl]thiazol-4-ylcarbonyl]-N′-[N-(8-quinolinoyl)-L-leucinyl]hydrazide;

N-[2-(N-cyclopropyl-N-cyclopropylmethylamino)thiazol-4-ylcarbonyl]-N′-[N-(7-quinolinoyl)-L-leucinyl]hydrazide;

N-[2-[N-bis-(cyclopropylmethyl)amino]thiazol-4-ylcarbonyl]-N′-[N-(6-methylnicotinoyl)-L-leucinyl]hydrazide;

N-[2-[N-bis-(cyclopropylmethyl)amino]thiazol-4-ylcarbonyl]-N′-(N-methyl-L-prolinyl-L-leucinyl)hydrazide;

N-(N-benzyloxycarbonyl-L-norvalinyl)-N′-[2-(1-naphthyl)thiazol-4-ylcarbonyl]hydrazide;

N-(N-benzyloxycarbonyl-L-isoleucinyl)-N′-[2-(1-naphthyl)thiazol-4-ylcarbonyl]hydrazide;

N-(N-benzyloxycarbonyl-L-norleucinyl)-N′-[2-(1-naphthyl)thiazol-4-ylcarbonyl]hydrazide;

N-[N-(4-dimethylaminomethylbenzyloxycarbonyl)-L-leucinyl]-N′-[2-(1-naphthyl)thiazol-4-ylcarbonyl]hydrazide;

N-(N-benzyloxycarbonyl-L-norvalinyl)-N′-[2-(2-benzyloxyphenyl)thiazol-4-ylcarbonyl]hydrazide;

N-[2-(N-cyclopropyl-N-cyclopropylmethylamino)thiazol-4-ylcarbonyl]-N′-[N-(4-methylimidazol-5-ylcarbonyl)-L-leucinyl]hydrazide;

N-[N-[4-(4-morpholinomethyl)benzoyl]-L-leucinyl]-N′-[2-(1-naphthyl)thiazol-4-ylcarbonyl]hydrazide;

N-[N-(6-methylnicotinoyl)-L-leucinyl]-N′-[2-(1-naphthyl)thiazol-4-ylcarbonyl]hydrazide;

N-(N-b-tert-butoxycarbonyl-L-tert-butylalanyl)-N′-[2-(N-cyclopropyl-N-cyclopropylmethylamino)thiazol-4-ylcarbonyl]hydrazide;

N-[2-(N-cyclopropyl-N-cyclopropylmethylamino)thiazol-4-ylcarbonyl]-N′-[N-(8-quinolinoyl)-L-b-tert-butylalanyl]hydrazide;

N-[N-(4-methylimidazol-5-ylcarbonyl)-L-allylglycinyl]-N′-[2-(1-naphthyl)thiazol-4-ylcarbonyl]hydrazide;

N-[N-(4-methylimidazol-5-ylcarbonyl)-L-b-tert-butylalanyl]-N′-[2-(1-naphthyl)thiazol-4-ylcarbonyl]hydrazide;

N-[2-(N-cyclopropyl-N-cyclopropylmethylamino)thiazol-4-ylcarbonyl]-N′-[N-(4-methylimidazol-5-ylcarbonyl)-L-b-tert-butylalanyl]hydrazide;

N-[2-(1-naphthyl)thiazol-4-ylcarbonyl]-N′-(N-picolinoyl-L-b-tert-butylalanyl)hydrazide;

N-[2-(1-naphthyl)thiazol-4-ylcarbonyl]-N′-[N-(8-quinolinoyl)-L-b-tert-butylalanyl]hydrazide;

N-[2-(1-naphthyl)thiazol-4-ylcarbonyl]-N′-(N-picolinoyl-L-allylglycinyl)hydrazide;

N-[2-(1-naphthyl)thiazol-4-ylcarbonyl]-N′-(N-picolinoyl-L-b-cyclopropylalanyl)hydrazide;

N-[N-(6-methylnicotinoyl-L-b-cyclopropylalanyl]-N′-[2-(1-naphthyl)thiazol-4-ylcarbonyl]hydrazide;

N-[N-(4-methylimidazol-5-ylcarbonyl)-L-b-cyclopropylalanyl]-N′-[2-(1-naphthyl)thiazol-4-ylcarbonyl]hydrazide;

N-[2-(1-naphthyl)thiazol-4-ylcarbonyl]-N′-[N-(8-quinolinoyl)-L-b-cyclopropylalanyl]hydrazide;

N-[N-(6-methylnicotinoyl)-L-b-tert-butylalanyl]-N′-[2-(1-naphthyl)thiazol-4-ylcarbonyl]hydrazide;

N-[2-(N-cyclopropyl-N-cyclopropylmethylamino)thiazol-4-ylcarbonyl]-N′-(N-picolinyl-L-b-tert-butylalanyl)hydrazide;

N-[2-(N-cyclopropyl-N-cyclopropylmethylamino)thiazol-4-ylcarbonyl]-N′-[N-(3-isoquinolinoyl)-L-b-tert-butylalanyl]hydrazide;

N-(N-tert-butoxycarbonyl-L-b-cyclopropylalanyl)-N′-[2-(N-cyclopropyl-N-cyclopropylmethylamino)thiazol-4-ylcarbonyl]hydrazide;

N-[2-(N-cyclopropylmethyl-N-propylamino)thiazol-4-ylcarbonyl]-N′-[N-(6-methyl-3-pyridinylmethoxycarbonyl)-L-leucinyl]hydrazide;

N-[N-(6-methylnicotinoyl)-L-allylglycinyl]-N′-[2-(1-naphthyl)thiazol-4-ylcarbonyl]hydrazide;

N-[2-(1-naphthyl)thiazol-4-ylcarbonyl]-N′-[N-(8-quinolinoyl)-L-allylglycinyl]hydrazide;

N-[2-(1-naphthyl)thiazol-4-ylcarbonyl]-N′-[N-(2-quinolinoyl)-L-b-cyclopropylalanyl]hydrazide;

N-[N-(3-isoquinolinoyl)-L-b-cyclopropylalanyl]-N′[-2-(1-naphthyl)thiazol-4-ylcarbonyl]hydrazide;

N-[N-(1-isoquinolinoyl)-L-b-cyclopropylalanyl]-N′-[2-(1-naphthyl)thiazol-4-ylcarbonyl]hydrazide;

N-[2-(1-naphthyl)thiazol-4-ylcarbonyl]-N′-[N-(7-quinolinoyl)-L-b-cyclopropylalanyl]hydrazide;

N-[2-(N-cyclopropyl-N-cyclopropylmethylamino)thiazol-4-ylcarbonyl]-N′-[N-(8-quinolinoyl)-L-b-cyclopropylalanyl]hydrazide;

N-[2-(N-cyclopropyl-N-cyclopropylmethylamino)thiazol-4-ylcarbonyl]-N′-[N-(4-methylimidazol-5-ylcarbonyl)-L-b-cyclopropylalanyl]hydrazide;

N-[2-(N-cyclopropyl-N-cyclopropylmethylamino)thiazol-4-ylcarbonyl]-N′-[N-(3-isoquinolinoyl)-L-b-cyclopropylalanyl]hydrazide;

N-[2-(N-cyclopropyl-N-cyclopropylmethylamino)thiazol-4-ylcarbonyl]-N′-[N-(6-methylnicotinoyl)-L-b-cyclopropylalanyl]hydrazide;

N-[N-(4-methylimidazol-5-ylcarbonyl)-L-norleucinyl]-N′-[2-(1-naphthyl)thiazol-4-ylcarbonyl]hydrazide;

N-[2-(1-naphthyl)thiazol-4-ylcarbonyl]-N′-(N-picolinoyl-L-norleucinyl)hydrazide;

N-[2-(1-naphthyl)thiazol-4-ylcarbonyl]-N′-[N-(8-quinolinoyl)-L-norleucinyl]hydrazide;

N-[2-(N-cyclopropyl-N-cyclopropylmethylamino)thiazol-4-ylcarbonyl]-N′-[N-(2-quinolinoyl)-L-b-cyclopropylalanyl]hydrazide;

N-[2-(N-cyclopropyl-N-cyclopropylmethylamino)thiazol-4-ylcarbonyl]-N′-[N-(1-isoquinolinoyl)-L-b-cyclopropylalanyl]hydrazide;

N-[2-[N-cyclopropyl-N-(2-methylpropyl)amino]thiazol-4-ylcarbonyl]-N′-[N-(6-methyl-3-pyridinylmethoxycarbonyl)-L-leucinyl]hydrazide;

N-(N-tert-butoxycarbonyl-L-leucinyl)-N′-[2-[N-cyclopropyl-N-(2-methylpropyl)amino]thiazol-4-ylcarbonyl]hydrazide;

N-[2-(1-naphthyl)thiazol-4-ylcarbonyl]-N′-[N-(7-quinolinoyl)-L-b-tert-butylalanyl]hydrazide;

N-[2-(1-naphthyl)thiazol-4-ylcarbonyl]-N′-[N-(2-quinolinoyl)-L-b-tert-butylalanyl]hydrazide;

N-[N-(1-isoquinolinoyl)-L-b-tert-butylalanyl]-N′-[2-(1-naphthyl)thiazol-4-ylcarbonyl]hydrazide;

N-[N-(3-isoquinolinoyl)-L-b-tert-butylalanyl]-N′-[2-(1-naphthyl)thiazol-4-ylcarbonyl]hydrazide;

N-[N-(6-methylnicotinoyl)-L-norleucinyl]-N′-[2-(1-naphthyl)thiazol-4-ylcarbonyl]hydrazide;

N-[2-(1-naphthyl)thiazol-4-ylcarbonyl]-N′-[N-(7-quinolinoyl)-L-norleucinyl]hydrazide;

N-[2-(1-naphthyl)thiazol-4-ylcarbonyl]-N′-[N-(2-quinolinoyl)-L-norleucinyl]hydrazide;

N-[N-(1-isoquinolinoyl)-L-norleucinyl]-N′-[2-(1-naphthyl)thiazol-4-ylcarbonyl]hydrazide;

N-[N-(3-isoquinolinoyl)-L-norleucinyl]-N′-[2-(1-naphthyl)thiazol-4-ylcarbonyl]hydrazide;

N-[2-(N-cyclopropyl-N-cyclopropylmethylamino)thiazol-4-ylcarbonyl]-N′-[N-(5-hydroxymethylimidazol-4-ylcarbonyl)-L-b-cyclopropylalanyl]hydrazide;

N-[2-[N-cyclopropyl-N-(2-methylpropyl)amino]thiazol-4-ylcarbonyl]-N′-[N-(8-quinolinoyl)-L-b-cyclopropylalanyl]hydrazide;

N-[2-(N-cyclopropyl-N-cyclopropylmethylamino)thiazol-4-ylcarbonyl]-N′-(N-(6-methylnicotinoyl)-L-b-tert-butylalanyl]hydrazide;

N-[2-[N-cyclopropyl-N-(2-methylpropyl)amino]thiazol-4-ylcarbonyl]-N′-[N-(4-methylimidazol-5-ylcarbonyl)-L-b-cyclopropylalanyl]hydrazide;

N-[2-[N-cyclopropyl-N-(2-methylpropyl)amino]thiazol-4-ylcarbonyl]-N′-[N-(2-quinolinoyl)-L-b-cyclopropylalanyl]hydrazide;

N-[2-[N-cyclopropyl-N-(2-methylpropyl)amino]thiazol-4-ylcarbonyl]-N′-[N-(6-methylnicotinoyl)-L-b-cyclopropylalanyl]hydrazide;

N-[2-(1-naphthyl)thiazol-4-ylcarbonyl]-N′-[N-(8-quinolinoyloglycinyl]hydrazide;

N-[2-(1-naphthyl)thiazol-4-ylcarbonyl]-N′-[N-(8-quinolinoyl)-L-norvalinyl]hydrazide;

N-[2-(1-naphthyl)thiazol-4-ylcarbonyl]-N′-[N-(2-quinolinoyl)-L-norvalinyl]hydrazide;

N-[2-(1-naphthyl)thiazol-4-ylcarbonyl]-N′-(N-picolinoyl-L-norvalinyl]hydrazide;

N-[2-(1-naphthyl)thiazol-4-ylcarbonyl]-N′-[N-(6-methylnicotinoyl)-L-norvalinyl]hydrazide;

N-[2-(1-naphthyl)thiazol-4-ylcarbonyl]-N′-[N-(4-methylimidazol-5-ylcarbonyl)-L-norvalinyl]hydrazide;

N-[2-(1-naphthyl)thiazol-4-ylcarbonyl]-N′-[N-(1-isoquinolinoyl)-L-norvalinyl]hydrazide;

N-[2-(1-naphthyl)thiazol-4-ylcarbonyl]-N′-[N-(3-isoquinolinoyl)-L-norvalinyl]hydrazide;

(1S, 1′S)-N,N′-bis-[4-[1-(N-benzyloxycarbonylamino)-3-methylbutyl]thiazol-2-ylcarbonyl]hydrazide;

N-[2-[N-cyclopropyl-N-(2-methylpropyl)amino]thiazol-4-ylcarbonyl]-N′-[N-(6-methylnicotinoyl)-L-b-tert-butylalanyl]hydrazide;

N-[2-[N-cyclopropyl-N-(2-methylpropyl)amino]thiazol-4-ylcarbonyl]-N′-[N-(4-methylimidazol-5-ylcarbonyl)-L-b-tert-butylalanyl]hydrazide;

N-[2-(N-cyclopropyl-N-cyclopropylmethylamino)thiazol-4-ylcarbonyl]-N′-[N-(1-isoquinolinoyl)-L-b-tert-butylalanyl]hydrazide;

N-[N-(5-butylpicolinoyl)-L-b-tert-butylalanyl]-N′-[2-(N-cyclopropyl-N-cyclopropylmethylamino)thiazol-4-ylcarbonyl]hydrazide;

N-[2-(N-cyclopropyl-N-cyclopropylmethylamino)thiazol-4-ylcarbonyl]-N′-[N-(6-methylpicolinoyl)-L-b-tert-butylalanyl]hydrazide;

N-[2-(N-cyclopropyl-N-cyclopropylmethylamino)thiazol-4-ylcarbonyl]-N′-[N-(4-fluorobenzoyl)-L-leucinyl]hydrazide;

N-[N-(4-fluorobenzoyl)-L-leucinyl]-N′-[2-(1-naphthyl)thiazol-4-ylcarbonyl]hydrazide;

N-[2-(1-naphthyl)thiazol-4-ylcarbonyl]-N′-[N-(2-pyridinylmethoxycarbonyl)-L-b-tert-butylalanyl]hydrazide;

N-[N-(2-methyl-3-pyridinylmethoxycarbonyl)-L-b-tert-butylalanyl]-N′-[2-(1-naphthyl)thiazol-4-ylcarbonyl]hydrazide;

N-[2-(1-naphthyl)thiazol-4-ylcarbonyl]-N′-[N-(2-pyridinylmethoxycarbonyl)-L-b-cyclopropylalanyl]hydrazide;

N-[N-(2-methyl-3-pyridinylmethoxycarbonyl)-L-b-cyclopropylalanyl]-N′-[2-(1-naphthyl)thiazol-4-ylcarbonyl]hydrazide;

N-[N-(6-methyl-3-pyridinylmethoxycarbonyl)-L-b-cyclopropylalanyl]-N′-[2-(1-naphthyl)thiazol-4-ylcarbonyl]hydrazide;

N-[N-(6-methyl-3-pyridinylmethoxycarbonyl)-L-b-tert-butylalanyl]-N′-[2-(1-naphthyl)thiazol-4-ylcarbonyl]hydrazide;

N,N′-bis-[2-(1-naphthyl)thiazol-4-ylcarbonyl]hydrazide;

N-[2-(N-cyclopropyl-N-cyclopropylmethylamino)thiazol-4-ylcarbonyl]-N′-[N-[2-(1,8-naphthyridinoyl)]-L-b-cyclopropylalanyl]hydrazide;

N-[2-[N-cyclopropyl-N-(2-methylpropyl)amino]thiazol-4-ylcarbonyl]-N′-[N-(3,4-difluorobenzoyl)-L-b-cyclopropylalanyl]hydrazide;

N-[2-[N-cyclopropyl-N-(2-methylpropyl)amino]thiazol-4-ylcarbonyl]-N′-[N-(4-flluorobenzoyl)-L-leucinyl]hydrazide;

N-[N-(5-butylpicolinoyl)-L-leucinyl]-N′-[2-(N-cyclopropyl-N-cyclopropylmethylamino)thiazol-4-ylcarbonyl]hydrazide;

N-[2-[N-cyclopropyl-N-(2-methylpropyl)amino]thiazol-4-ylcarbonyl]-N′-[N-(3,4-dimethoxybenzoyl)-L-leucinyl]hydrazide;

N-[2-[N-cyclopropyl-N-(2-methylpropyl)amino]thiazol-4-ylcarbonyl]-N′-[N-(3,4-difluorobenzoyl)-L-b-tert-butylalanyl]hydrazide;

N-[2-[N-cyclopropyl-N-(2-methylpropyl)amino]thiazol-4-ylcarbonyl]-N′-[N-(3,4-dimethoxybenzoyl)-L-b-tert-butylalanyl]hydrazide;

N-[N-(5-butylpicolinoyl)-L-b-tert-butylalanyl]-N′-[2-(N-cyclopropyl-N-cyclopropylmethylamino)thiazol-4-ylcarbonyl]hydrazide; and

N-[2-[N-cyclopropyl-N-(2-methylpropyl)amino]thiazol-4-ylcarbonyl]-N′-[N-(6-methylpicolinoyl)-L-b-tert-butylalanyl]hydrazide.

### Definitions

The present invention includes all hydrates, solvates, complexes and prodrugs of the compounds of this invention. Prodrugs are any covalently bonded compounds which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984). The term “amino acid” as used herein refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

“C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. Any C₁₋₆alkyl group may be optionally substituted independently by one or two halogens, SR′, OR′, N(R′)₂, C(O)N(R′)₂, carbamyl or C₁₋₄alkyl, where R′ is C₁₋₆alkyl. C₀alkyl means that no alkyl group is present in the moiety. Thus, Ar—C₀alkyl is equivalent to Ar.

“C₃₋₁₁cycloalkyl” as applied herein is meant to include substituted and unsubstituted cyclopropane, cyclobutane, cyclopentane, cyclohexane, cycloheptane, cyclooctane, cyclononane, cyclodecane, cycloundecane. When substituted, substituents are defined as for “C₁₋₆alkyl”, above.

“C₂₋₆alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

“C₂₋₆alkynyl” means an alkyl group of 2 to 6 carbons wherein one carbon-carbon single bond is replaced by a carbon-carbon triple bond. C₂₋₆alkynyl includes acetylene, 1-propyne, 2-propyne, 1-butyne, 2-butyne, 3-butyne and the simple isomers of pentyne and hexyne.

“Halogen” means F, Cl, Br, and I.

“Ar” or “aryl” or “Ar′” or “aryl′” means phenyl or naphthyl, optionally independently substituted by one or more of Ph—C₀₋₆alkyl, Het—C₀₋₆alkyl, C₁₋₆alkyl, C₁₋₆alkoxy, Ph—C₀₋₆alkoxy, Het—C₀₋₆alkoxy, OH, (CH₂)₁₋₆NR⁸R⁹, O(CH₂)₁₋₆NR⁸R⁹, CO₂R′, or halogen. Two C₁₋₆alkyl groups may be combined to form a 5-7 membered ring, saturated or unsaturated, fused onto the Ar ring. Ph may be optionally substituted with one or more of C₁₋₆alkyl, C₁₋₆alkoxy, OH, (CH₂)₁₋₆NR⁸R⁹, O(CH₂)₁₋₆NR⁸R⁹, CO₂R′, or halogen.

As used herein “Het” or “heterocyclic” represents a stable 5- to 7-membered monocyclic or a stable 7- to 10-membered bicyclic heterocyclic ring, which is either saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from the group consisting of Ph—C₀₋₆alkyl, Het—C₀₋₆ alkyl, C₁₋₆alkyl, C₁₋₆alkoxy, Ph—C₀₋₆alkoxy, Het—C₀₋₆alkoxy, OH, (CH₂)₁₋₆ NR⁸R⁹, O(CH₂)₁₋₆NR⁸R⁹, CO₂R′. Two C₁₋₆alkyl groups may be combined to form a 5-7 membered ring, saturated or unsaturated, fused onto the Het ring. Ph may be optionally substituted with one or more of C₁₋₆alkyl, C₁₋₆alkoxy, OH, (CH₂)₁₋₆NR⁸R⁹, O(CH₂)₁₋₆NR⁸R⁹, CO₂R′, or halogen. Examples of such heterocycles include the piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, thiamorpholinyl sulfoxide, thiamorpholinyl sulfone, and oxadiazolyl rings.

“HetAr” or “heteroaryl” means any heterocyclic moiety encompassed by the above definition of Het which is aromatic in character, e.g., pyridine.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. EDC refers to N-ethyl-N′(dimethylaminopropyl)-carbodiimide. HOBT refers to 1-hydroxybenzotriazole, DMF refers to dimethyl formamide, BOP refers to benzotriazol-1-yloxy-tris(dimethylamino)phosphonium hexafluorophosphate, Lawesson's reagent is 2,4-bis(4-methoxyphenyl)-1,3-dithia-2,4-diphosphetane-2,4-disulfide, NMM is N-methylmorpholine, TFA refers to trifluoroacetic acid, and THF refers to tetrahydrofuran.

### Methods of Preparation

Compounds of the Formula I wherein X=S, Y=CH, Z=N and L=NR⁴R⁷, are prepared by methods analogous to those described in Scheme 1.

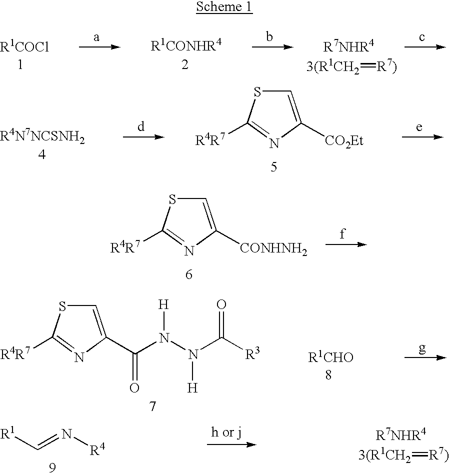

a) R⁴NH₂, Py, CH₂Cl₂; b) LiAlH₄, THF; c) i. Cl₂CS, Py, CH₂Cl₂; ii. NH₃, MeOH or I, PhCONCS, CHCl₃; ii. K₂CO₃, MeOH, H₂O; d) EtO₂CCOCH₂Br, EtOH; e) H₂NNH₂.H₂O, EtOH; f) R³CO₂H, EDC.HCl, 1-HOBT, DMF or R¹¹R¹²NCOCl, Et₃ N, CH₂Cl₂ where W is C(O), or R³SO₂Cl, NMM, CH₂Cl₂ where W is SO₂; g) R⁴NH₂, CH₂Cl₂; h)LiAlH₄, Et₂O; j) Na(OAc)₃BH, CH₂Cl₂.

An acid chloride (such as cyclopropanecarbonyl chloride or isobutyryl choride) (1-Scheme 1) is treated with a primary amine (such as aniline, cyclopropylamine, isobutylamine or propylamine) and pyridine in an aprotic solvent (such as methylene chloride) to provide 2-Scheme 1, which is treated with lithium aluminum hydride in THF to afford 3-Scheme 1. Alternatively, 3-Scheme 1 may be prepared by treatment of an aldehyde (such as cyclopropanecarboxaldehyde or isobutyraldehyde) (8-Scheme 1) with an amine (such as cyclopropylamine) in methylene chloride to provide 9-Scheme 1, which is treated with a reducing agent (such as lithim aluminum hydride in ether or sodium triacetoxyborohydride in methylene chloride). Treatment of 3-Scheme 1 with thiophosgene and pyridine in methylene chloride, followed by treanment with ammonia in methanol provides 4-Scheme 1. Alternatively, 4-Scheme 1 may be prepared by treatment of 3-Scheme 1 with benzoyl isothiocyanate, followed by treatment of the intermediate benzoyl thiourea with potassium carbonate in methanol/water. 4-Scheme 1 is treated with hydrazine hydrate in ethanol to give 5-Scheme 1. Treatment of 5-Scheme 1 with a carboxylic acid (such as N-(2-pyridinylmethoxycarbonyl)-L-leucine, N-(3-pyridinylmethoxycarbonyl)-L-leucine, N-(4-pyridinylmethoxycarbonyl)-L-leucine, N-methyl-N-(4-pyridinylmethoxycarbonyl)-L-leucine, N-methyl-N-(3-pyridinylmethoxycarbonyl)-L-leucine, N-methyl-N-(2-pyridinylmethoxycarbonyl)-L-leucine, 4-methyl-2-(3-phenylphenyl)pent-4enoic acid, 4-methyl-2-(3-phenylphenyl)pentanoic acid, N-tert-butoxycarbonyl-L-leucine, N-(4-tert-butoxycarbonylbenzyloxycarbonyl)-L-leucine, N-(6-methyl-3-pyridinylmethoxycarbonyl)-L-leucine, N-(2-methyl-3-pyridinylmethoxycarbonyl)-L-leucine, N-(4-tert-butoxycarbonylbenzyloxycarbonyl)-L-leucine, N-tert-butoxycarbonyl-L-b-tert-butylalanine and N-tert-butoxycarbonyl-L-b-cyclopropylalanine) and a peptide coupling reagent (such as EDC.HCl/1-HOBT) in an aprotic solvent (such as DMF) or with a carbamoyl chloride (such as N,N-diisobutylcarbamoyl chloride) and triethylamine in methylene chloride affords 6-Scheme 1.

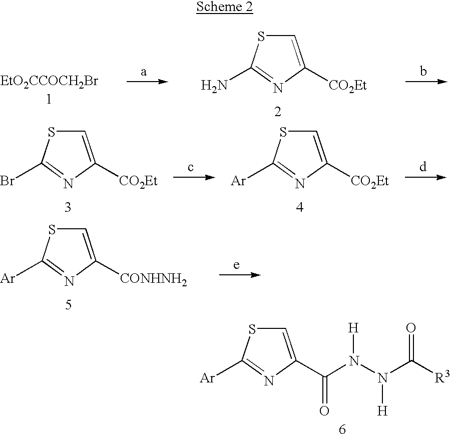

a) Thiourea, EtOH; b) i. NaNO₂, 16% aqueous HBr; ii. CuBr, 16% aqueous HBr; iii. HBr (cat.), EtOH; c) ArB(OH)₂, Pd(PPh₃)₄, NaHCO₃, toluene, EtOH, H₂O; d) H₂NNH₂.H₂O, EtOH; e) R³CO₂H, EDC.HCl, 1-HOBT, DMF where W is C(O), or R³SO₂Cl, NMM, CH₂Cl₂ where W is SO₂.

Compounds of the Formula I wherein X=S, Y=CH, Z=N and L=Ar or Het, are prepared by methods analogous to those described in Scheme 2. Ethyl bromopyruvate (1-Scheme 2) is treated with thiourea in refluxing ethanol to provide 2-Scheme 2 which is treated successively with sodium nitrite and copper (I) bromide in 16% aqueous HBr, and the product was heated in ethanol with a catalytic amount of HBr to give 3-Scheme 2. Treatment of this material with an arylboronic acid (such as 2-benzyloxyphenylboronic acid, 1-naphthylboronic acid, 4-methyl1-naphthylboronic acid, 5-acenaphthylboronic acid, 2-methoxy-1-naphthylboronic acid, 2-methoxymethoxy-1-naphthylboronic acid, 9-anthracenylboronic acid, 9-phenanthenylboronic acid, 2-(4-tert-butoxycarbonylbenzyloxy)phenylboronic acid, 4-methoxymethoxynaphthylboronic acid or 8-quinolineboronic acid), tetrakis(triphenylphosphine)palladium(0) and sodium bicarbonate in refluxing toluenelethaonlwater provides 4-Scheme 2. Treatment of 4-Scheme 2 with hydrazine hydrate in ethanol provides 5-Scheme 2, which is treated with a carboxylic acid (such as N-(2-pyridinylmethoxycarbonyl)-L-leucine, N-(3-pyridinylmethoxycarbonyl)-L-leucine, N-(4-pyridinylmethoxycarbonyl)-L-leucine, N-methyl-N-(3-pyridinylmethoxycarbonyl)-L-leucine, N-benzyloxycarbonyl-L-leucine, 4-methyl-2-(3-phenylphenyl)pentanoic acid, 4-methyl-2-(3-phenoxyphenyl)pentanoic acid, 4-methyl-2-(4-phenoxyphenyl)pentanoic acid, N-benzyloxycarbonyl-L-b-tert-butylalanine, N-benzyloxycarbonyl-L-b-cyclopropylalanine, N-benzyloxycarbonyl-L-norvaline, N-benzyloxycarbonyl-L-norleucine, N-benzyloxycarbonyl-L-isoleucine, N-(4-dimethylaminomethylbenzyloxycarbonyl-L-leucine), N-tert-butoxycarbonyl-L-leucine, N-(6-methyl-3-pyridinylmethoxycarbonyl)-L-leucine, N-(2-methyl-3-pyridinylmethoxycarbonyl)-L-leucine, N-(8-quinolinoyl)-L-leucine, N-(8-quinolinoyl)glycine, N-tert-butoxycarbonyl-L-allylglycine, N-tert-butoxycarbonyl-L-norleucine, N-tert-butoxycarbonyl-L-norvaline, N-tert-butoxycarbonyl-L-b-tert-butylalanine, N-tert-butoxycarbonyl-L-b-cyclopropylalanine) and a peptide coupling reagent (such as EDC.HCl/1-HOBT) in an aprotic solvent (such as DMF) or with a carbamoyl chloride (N-isobutyl-N-phenylcarbamoyl chloride) and triethylamine in methylene chloride to provide 6-Scheme 2 where W is C(O). Where W═SO₂, 5-Scheme 2 is treated with a corresponding sulfonyl chloride, R³SO₂Cl, and n-methylmorpholine (NMM) in methylene chloride.

Compounds of the Formula I wherein X=S, Y=CH and Z=N, are prepared by methods analogous to those described in Scheme 1.

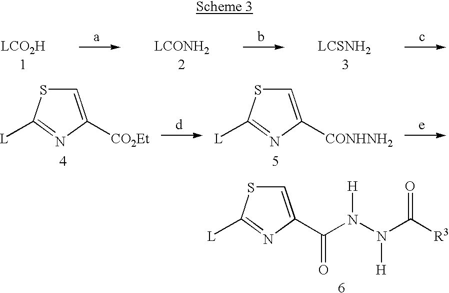

a) i-BuOCOCl, NMM, NH₃, TBF; b) Lawesson's reagent, THF; c) i. EtO₂CCOCH₂Br; ii. TFAA, Py, CH₂Cl₂; d) H₂NNH₂.H₂O, EtOH; e) R³CO₂H, EDC.HCl, 1-HOBT, DMF where W is C(O), or R³SO₂Cl, NMM, CH₂Cl₂ where W is SO₂.

A carboxylic acid (such as N-benzyloxycarbonyl-L-leucine) (1-Scheme 3) is converted to 2-Scheme 3 by treatment with isobutyl chloroformate, N-methylmorpholine and ammonia in TBF. 2-Scheme 3 is treated with Lawesson′s reagent in THF to provide the thioamide 3-Scheme 3. This material is converted to the thiazole by condensation with an a-ketoester followed by treatment with trifluoroacetic anhydride and pyridine in methylene chloride to afford 4-Scheme 3 which is converted to 5-Scheme 3 by treatment with hydrazine monohydrate. This material is treated with a carboxylic acid (such as (1S)-1-benzyloxycarbonylamino-1-(4-carboxythiazol-2-yl)-3-methylbutane) and a peptide coupling reagent (such as EDC.HCl/1-HOBT) in an aprotic solvent (such as DMF) to provide 6-Scheme 3 where W is C(O). Where W=SO₂, 5-Scheme 3 is treated with a corresponding sulfonyl chloride, R³SO₂Cl, and n-methylmorpholine (NMM) in methylene chloride.

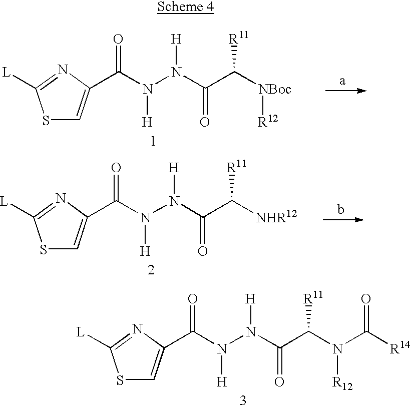

a) TFA; b) R¹⁴CO₂H, EDC.HCl, 1-HOBT, DMF.

Compounds of the Formula I wherein X=S, Y=CH, Z=N, R³=CH(R¹¹)NR¹²R¹³ where R¹³=R¹⁴CO are prepared by methods analogous to those described in Scheme 4. 1-Scheme 4 is treated with trifluoroacetic acid to provide 2-Scheme 4. This material is treated with a carboxylic acid (such as pryazinecarboxylic acid, picolinic acid, 2-quinolinecarboxylic acid, 3-quinolinecarboxylic acid, 4-quinolinecarboxylic acid, 5-quinolinecarboxylic acid, 6-quinolinecarboxylic acid, 7-quinolinecarboxylic acid, 8-quinolinecarboxylic acid, 1-isoquinolinecarboxylic acid, 3-isoquinolinecarboxylic acid, N-methylpiperidinecarboxlic acid, 4-methylimidazole-5-carboxylic acid, N-benzylproline, N-methylproline, 1-benzyl-5-methylimidazole 4-carboxylic acid, 6-methylnicotinic aicd, 2-methylnicotinic acid, 2-methylisonicotinic acid, 4-dimethyaminomethylbenzoic acid, 4-(4-morpholino)benzoic acid, 5-hydroxymethylimidazole-4-carboxylic acid, 5-butylpicolinic acid or 4-fluorobenzoic acid) and a peptide coupling reagent (such as EDC.HCl/1-HOBT) in an aprotic solvent (such as DMF) to provide 3-Scheme 4.

The starting materials used herein are commercially available amino acids or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well known to the art. The methods of peptide synthesis generally set forth by Bodansky et al., THE PRACTICE OF PEPTIDE SYNTHESIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIDE SYNTHESIS, 2d Ed., Pierce Chemical Co., Rockford, Ill., 1984, are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

This invention also provides a pharmaceutical composition which comprises a compound according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

### Utility of the Present Invention

The compounds of Formula I are useful as protease inhibitors, particularly as inhibitors of cysteine and serine proteases, more particularly as inhibitors of cysteine proteases, even more particularly as inhibitors of cysteine proteases of the papain superfamily, yet more particularly as inhibitors of cysteine proteases of the cathepsin family, most particularly as inhibitors of cathepsin K. The present invention also provides useful compositions and formulations of said compounds, including pharmaceutical compositions and formulations of said compounds.

The present compounds are useful for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy; and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease; hypercalcemia of malignancy, and metabolic bone disease.

Metastatic neoplastic cells also typically express high levels of proteolytic enzymes that degrade the surrounding matrix, and certain tumors and metastatic neoplasias may be effectively treated with the compounds of this invention.

The present invention also provides methods of treatment of diseases caused by pathological levels of proteases, particularly cysteine and serine proteases, more particularly cysteine proteases, even more particularly cysteine proteases of the papain superfamily, yet more particularly cysteine proteases of the cathepsin family, which methods comprise administering to an animal, particularly a mammal, most particularly a human, in need thereof an effective amount of a compound or combination of compounds of the present invention. The present invention especially provides methods of treatment of diseases caused by pathological levels of cathepsin K, which methods comprise administering to an animal, particularly a mammal, most particularly a human, in need thereof an effective amount of an inhibitor of cathepsin K, including a compound or combination of compounds of the present invention. The skilled artisan will understand that by the term “effective amount” is meant that amount of a compound or combination of compounds of the present invention sufficient to ameliorate or cure the clinically undesirable manifestations of disease (e.g. brittle and weakened bone in osteoporosis) caused by said pathological levels of target enzyme, e.g., cathepsin K, by inhibition of the target enzyme. The present invention particularly provides methods for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy, and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease, hypercalcemia of malignancy, and metabolic bone disease.

This invention further provides a method for treating osteoporosis or inhibiting bone loss which comprises internal administration to an animal, particularly a mammal, most particularly a human in need thereof an effective amount of a compound or combination of compounds of Formula I, alone or in combination with other inhibitors of bone resorption, such as bisphosphonates (i.e., allendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

### Biological Assays

The compounds of the present invention may be tested in one of several biological assays to determine the concentration of compound which is required to provide a given pharmacological effect.

Determination of Cathepsin K Proteolytic Catalytic Activity

All assays for cathepsin K were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 mM EDTA. Stock substrate solutions were prepared at concentrations of 10 or 20 mM in DMSO with 20 uM final substrate concentration in the assays. All assays contained 10% DMSO. Independent experiments found that this level of DMSO had no effect on enzyme activity or kinetic constants. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):

v=VₘA/[Kₐ(1+I/K_{i,app})+A] (1)

where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:

[AMC]=vₛₛt+(v₀−vₛₛ)[1−exp(−k_{obs}t)]/k_{obs} (2)

where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol., 1988, 61, 201).

Human Osteoclast Resorption Assay

Aliquots of osteoclastoma-derived cell suspensions were removed from liquid nitrogen storage, warmed rapidly at 37° C. and washed ×1 in RPMI-1640 medium by centrifugation (1000 rpm, 5 min at 4° C.). The medium was aspirated and replaced with murine anti-HLA-DR antibody, diluted 1:3 in RPMI-1640 medium, and incubated for 30 min on ice. The cell suspension was mixed frequently.

The cells were washed ×2 with cold RPMI-1640 by centrifugation (1000 rpm, 5 min at 4° C.) and then transferred to a sterile 15 mL centrifuge tube. The number of mononuclear cells were enumerated in an improved Neubauer counting chamber.

Sufficient magnetic beads (5/mononuclear cell), coated with goat anti-mouse IgG, were removed from their stock bottle and placed into 5 mL of fresh medium (this washes away the toxic azide preservative). The medium was removed by immobilizing the beads on a magnet and is replaced with fresh medium.

The beads were mixed with the cells and the suspension was incubated for 30 min on ice. The suspension was mixed frequently. The bead-coated cells were immobilized on a magnet and the remaining cells (osteoclast-rich fraction) were decanted into a sterile 50 mL centrifuge tube. Fresh medium was added to the bead-coated cells to dislodge any trapped osteoclasts. This wash process was repeated ×10. The bead-coated cells were discarded.

The osteoclasts were enumerated in a counting chamber, using a large-bore disposable plastic pasteur pipette to charge the chamber with the sample. The cells were pelleted by centrifugation and the density of osteoclasts adjusted to 1.5×10⁴/mL in EMEM medium, supplemented with 10% fetal calf serum and 1.7 g/liter of sodium bicarbonate. 3 mL aliquots of the cell suspension (per treatment) were decanted into 15 mL centrifuge tubes. These cells were pelleted by centrifugation. To each tube 3 mL of the appropriate treatment was added (diluted to 50 uM in the EMEM medium). Also included were appropriate vehicle controls, a positive control (87MEM1 diluted to 100 ug/mL) and an isotype control (IgG2a diluted to 100 ug/mL). The tubes were incubate at 37° C. for 30 min.

0.5 mL aliquots of the cells were seeded onto sterile dentine slices in a 48-well plate and incubated at 37° C. for 2 h. Each treatment was screened in quadruplicate. The slices were washed in six changes of warm PBS (10 mL/well in a 6-well plate) and then placed into fresh treatment or control and incubated at 37° C. for 48 h. The slices were then washed in phosphate buffered saline and fixed in 2% glutaraldehyde (in 0.2M sodium cacodylate) for 5 min., following which they were washed in water and incubated in buffer for 5 min at 37° C. The slices were then washed in cold water and incubated in cold acetate buffer/fast red garnet for 5 min at 4° C. Excess buffer was aspirated, and the slices were air dried following a wash in water.

The TRAP positive osteoclasts were enumerated by bright-field microscopy and were then removed from the surface of the dentine by sonication. Pit volumes were determined using the Nikon/Lasertec ILM21W confocal microscope.

General

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

Where indicated, certain of the materials were purchased from the Aldrich Chemical Co., Milwaukee, Wis., Chemical Dynamics Corp., South Plainfield, N.J., and Advanced Chemtech, Louisville, Ky.

### EXAMPLES

In the following synthetic examples, temperature is in degrees Centigrade (°C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

### Preparation of N-[2-(cis-2,6-dimethyl-4-morpholino)thiazol-4-ylcarbonyl]-N′-[N-(4-pyridinylmethoxycarbonyl)-L-leucinyl]hydrazide

### a) cis-2,6-dimethyl-4-morpholino-N-benzoylthiourea

Cis-2,6-dimethylmorpholine (1.40 g, 12.17 mmol, 1.5 mL) was dissolved in chloroform (20 mL) and benzoyl isothiocyanate (2.0 g, 12.17 mmol, 1.75 mL) was added. After stirring 45 minutes at room temperature, the solution was concentrated to giv the title compound as a yellow solid (3.94 g, 100%). MS (ESI): 279.2 (M+H)⁺.

### b) cis-2,6dimethyl-4-morpholinothiourea

The compound of Example 1(a) (3.38 g, 12.17 mmol) was dissolved in methanol (40 mL) and water (40 mL), potassium carbonate (8.4 g, 60.84 mmol) was added and the solution was heated at reflux overnight. The reaction mixture was concentrated, redissolved in ethyl acetate, washed with sodium bicarbonate and water, then dried (MgSO₄), filtered and concentrated to afford the title compound as a beige solid (1.7 g, 80%). MS (ESI): 174.9 (M+H)⁺.

### c) ethyl 2-(cis-2,6-dimethyl-4-morpholino)thiazole-4-carboxylate

The compound of Example 1(b) (1.7 g, 9.74 mmol) was dissolved in ethanol (25 mL) upon heating. The solution was cooled to room temperature and ethylbromopyruvate (1.22 mL, 9.74 mmol) was added. The reaction mixture was heated at reflux for 10 minutes, then concentrated. The residue was partitioned between ethyl acetate and saturated aqueous sodium bicarbonate. The aqueous phase was extracted with ethyl acetate and the combined organic phases were washed with saturated brine, dried (MgSO₄), filtered and concentrated to an orange oil. The crude product was passed trough silica gel eluting with ethyl acetate/hexane (1:8, then 1:3) to give the title compound as a yellow solid (2.07 g, 79%). MS (ESI): 271.3 (M+H)⁺.

### d) N-[2-(cis-2,6-dimethyl-4-morpholino)thiazol-4-ylcarbonyl]hydrazide

The compound of Example 1(c) (2.07 g, 7.65 mmol) was dissolved in ethanol (25 mL) and hydrazine monohydrate (3.7 mL, 76.56 mmol) was added. The solution was heated at reflux for 2 hours, then concentrated to afford the title compound as an orange solid (1.96 g, 100%). MS (ESI): 257.2 (M+H)⁺.

### e) a-isocyanato-L-leucine Methyl Ester

L-leucine methyl ester hydrochloride (25 g, 0.14 mol) was dissolved in methylene chloride (450 mL), cooled to 0° C., and pyridine (43.5 g, 0.55 mol, 44.5 mL) was added, then a 1.93 M solution of phosgene in toluene (0.18 mol, 92.7 ml) was added slowly. After stirring at 0° C. for 2 h, the mixture was poured into 0.5 N HCl (1400 mL) and ice (900 mL). The organic layer was washed with 0.5 N HCl (1400 mL) and ice (900 mL). The aqueous layers were extracted with methylene chloride (450 mL) and the combined organic layers were washed with saturated brine (1400 mL) and ice (900 mL), then dried (MgSO₄), filtered and concentrated. The residue was distilled (56-58° C.; 0.78 mmHg) to provide the title compound as a colorless liquid (20.4 g, 86%). ¹H NMR (250 MHz, CDCl₃) d 4.04 (dd, 1H), 3.82 (s, 3H), 1.92-1.72 (m, 1H), 1.69-1.62 (m, 2H), 0.96 (d, 3H), 0.94 (d, 3H).

### f) N-(4-pyridinylmethoxycarbonyl)-L-leucine Methyl Ester

A solution of the compound of Example 1(e) (5.10 g, 29.8 mmol) and 4-pyridylcarbinol (3.25 g, 29.8 mmol) in toluene (30 mL) was heated at reflux for 24 h. The solution was concentrated and the residue was purified by flash chromatography on 250 g of 230-400 mesh silica gel, eluting with 3:1 ethyl acetate/hexanes, to give the title compound (7.86 g, 94%). ¹H NMR (250 MHz, CDCl₃) d 8.59 (d, 2H), 7.24 (d, 2H), 5.33 (d, 1H), 5.13 (s, 3H), 4.40 (dt, 1H), 3.75 (s, 3H), 1.81-1.51 (m, 3H), 0.96 (d, 3H), 0.95 (d, 3H).

### g) N-(4-pyridinylmethoxycarbonyl)-L-leucine

To a stirring solution the compound of Example 1(f) (1.98 g, 7.06 mmol) in THF (7 mL) was added 7 mL of water followed by LiOH.H₂O (325 mg, 7.76 mmol). The mixture was stirred for 30 minutes and then concentrated. The residue was redissolved in water (10 mL) and 3 N HCl was added (2.6 mL). The solution was lyophilized to yield a white solid (2.015 g, 6.44 nmuol). MS (ESI): 267.2 (M+H)⁺.

### h) N-[2-(cis-2,6-dimethyl-4-morpholino)thiazol-4-ylcarbonyl]-N′-[N-(4-pyridinylmethoxycarbonyl)-L-leucinyl]hydrazide

To a stirring solution of the compound of Example 1(g) (104 mg, 0.39 mmol) in DMF (2.5 mL) was added the compound of Example 1(d) (100 mg, 0.39 mmol), 1-hydroxybenzotriazole (9.5 mg, 0.07 mmol), and 1-(3-dimethylaminopropyl)-3-ethylcarbodiimide hydrochloride (100 mg, 0.39 mmol). After stirring at room temperature for 16 h, the solution was partitioned between ethyl acetate and water. The aqueous layer was extracted with ethyl acetate. The combined organic layers were washed with saturated brine, dried (MgSO₄), filtered and concentrated. The crude product was purified by column chromatography on silica gel (6% methanol in methylene chloride) to afford the title compound as a white solid (125 mg, 51%). MS (ESI): 505.4 (M+H)⁺.

### Example 2

### Preparation of N-[2-[N-cyclopropylmethyl-N-(2-methylpropyl)amino]thiazol-4-ylcarbonyl]-N′-[N-(4-pyridinylmethoxycarbonyl)-L-leucinyl]hydrazide

### a) N-cyclopropylmethyl Isobutyramide

Triethylamine (1.53 g, 15.09 mmol, 2.1 mL) and isobutylamine (1.10 g, 15.09 mmol, 1.5 mL) were dissolved in methylene chloride (15 mL), cooled to 0° C., and cyclopropane carbonyl chloride (1.58 g, 15.09 mmol, 1.4 mL) was added dropwise. After stirring at 0° C. for one hour the mixture was diluted with methylene chloride (60 mL) and washed with NaOH (1M), then with saturated brine, dried (MgSO₄), filtered and concentrated. The residue was washed with ether and dried to give the title compound as a beige solid (2.1 g, 100%). MS (ESI): 141.9 (M+H)⁺.

### b) N-cyclopropylmethyl Isobutylamine

To a stirring solution of 1M LiAlH₄ in THF (11.3 mL, 11.3 mmol), cooled to 0° C., was added slowly over 20 minutes a solution of the the compound of Example 2(a) (1.595 g, 11.3 mmol) in THF (20 mL). After the addition was complete, the ice bath was removed and the solution was heated at 55° C. for 30 minutes. The mixture was cooled to 0° C. and quenched with water (0.43 mL) and 15% aqueous NaOH (0.43 mL) and water (1.29 mL). The solid was removed by filtration and washed with ether, dried (MgSO₄) and filtered. The filtrate was evaporated to dryness to give the title compound as a a colorless liquid (1.15 g, 80%). MS (ESI): 128.0 (M+H)⁺.

### c) N-[2-[N-cyclopropylmethyl-N-(2-methylpropyl)amino]thiazol-4-ylcarbonyl]-N′-[N-(4-pyridinylmethoxycarbonyl)-L-leucinyl]hydrazide

Following the procedure of Example 1(a)-1(h), except substituting N-cyclopropylmethyl isobutylamine for cis-2,6-dimethylmorpholine in step (a), the title compound was prepared as a yellow solid (60 mg, 31%). MS (ESI): 517.3 (M+H)⁺.

### Example 3

### Preparation of N-[2-(4-methyl-1-naphthyl)thiazol-4-ylcarbonyl]-N′-[N-(4-pyridinylmethoxycarbonyl)-L-leucinyl]hydrazide

### a) ethyl 2-aminothiazole-4-carboxylate Hydrobromide

To a stirring suspension of thiourea (46.7 g, 0.614 mol) in EtOH (640 mL) was added ethyl bromopyruvate (120 g, 0.614 mol, 77.2 mL) slowly. After stirring at 45° C. for 16 h the solution was cooled to room temperature and placed in the refrigerator overnight. The mixture was filtered, the crystals were washed with cold ethanol and air dried to give the product as pale yellow crystals (132.74 g, 85%). MS (ESI): 172.9 (M+H)⁺.

### b) 2-bromothiazole-4-carboxylic Acid

To a stirring suspension of the compound of Example 3(a) (32.11 g, 0.127 mol) in 16% HBr (aq) (400 mL) at 0° C. a solution of NaNO₂ (9.11 g, 0.132 mol) in water (16 mL) was added. After stirring for 35 min, CuBr (20.6 g, 0.144 mol) was added followed by additional 16% Hbr(aq) (150 mL). The mixture was heated at 70° C. for 1 h and immediately filtered. The filtrate was saturated with NaCl and extracted with ethyl acetate (2×500 mL). The organic phases were combined, dried (MgSO₄), filtered and concentrated to a brown solid. This was combined with solid collected by filtration and used without further purification or characterization in the next step.

### c) ethyl 2-bromothiazole-4-carboxylate

The compound of Example 1(b) was heated at reflux in EtOH (1 L) for 1 h, then filtered. To the filtrate was added 64 drops of 48% (aq) HBr. After stirring at reflux for 24 h the solution was concentrated and redissolved in EtOAc (1 L). The solution was washed successively with saturated aqueous NaHCO₃ (1 L) and brine (1 L), dried (MgSO₄), filtered, decolorized with charcoal, filtered through Celite, and concentrated to a pale yellow solid (16.95 g, 56%). ¹H NMR (400 MHz, CDCl₃) d 8.14 (s, 1H), 4.46, (q, 2H), 1.43 (t, 3H).

### d) 4-methyl-L-naphthalene Boronic Acid

To a stirring solution of 1-bromo-4-methylnaphthalene (1.0 g, 4.52 mmol) in THF (5 mL) at −78° C. was added N-butyllithium (1.8 mL, 4.52 mrnol, 2.5M in hexane) dropwise. After stirring at −78° C. for 1 h, triisopropylborate (4.52 g, 22.6 mmol) was added. After stirring at room temperature for 3 h, the solution was partitioned between 3N HCl and ethyl acetate. The organic phase was washed successively with saturated aqueous NaHCO₃ and brine, then dried (MgSO₄), filtered and concentrated to a yellow solid which was washed with hexane to yield the title compound as a pale yellow solid (0.5 g, 59%). ¹H NMR (400 MHz, CDCl₃) d 9.35 (d, 1H), 8.58 (d, 1H), 8.14 (d, 1H), 7.64 (m, 2H), 7.54 (d, 1H), 2.82, (s, 3H).

### e) ethyl 2-(4-methyl-1-naphthyl)thiazol-4-carboxylate

To a stirring mixture of the compound of Example 1(c) (0.30 g, 1.27 mmol), the compound of Example 1(d) (0.355 g, 1.91 mmol), and Pd(Ph₃P)₄ (0.059 g, 0.05 mmol)) in EtOH (4 mL) and toluene (4 mL) was added NaHCO₃ (4.42 mL, 1.0 M in water). After stirring at reflux for 4 h,the mixture was cooled and partitioned between 1 N HCl (25 mL) and ethyl acetate (25 mL). The organic layer was washed with brine, dried (MgSO₄), filtered and concentrated. The residue was purified by column chromatography (silica gel, ethyl acetate/hexane) to yield the title compound as a foamy solid. (0.257 g, 68%). MS (ESI): 298.2 (M+H)⁺.

### f) N-[2-(4-methyl-1-naphthyl)thiazol-4-ylcarbonyl]hydrazide

Following the procedure of Example 1(d), except substituting ethyl 2-(4-methyl-1-naphthyl)thiazole-4-carboxylate for ethyl 2-(cis-2,6-dimethyl-4-morpholino)thiazole-4-carboxylate, the title compound was prepared as a pale yellow solid (0.245 g, 100%). MS (ESI): 284.2 (M+H)⁺.

### g) N-[2-(4-methyl-1-naphthyl)thiazol-4-ylcarbonyl]-N′-[N-(4-pyridinylmethoxycarbonyl)-L-leucinyl]hydrazide

Following the procedure of Example 1(e)-1(h), except N-[2-(4-methyl-1-naphthyl)thiazol-4-ylcarbonyl]hydrazide for N-[2-(cis-2,6-dimethyl-4-morpholino)thiazol-4-ylcarbonyl]hydrazide in step (h), the title compound was prepared as a white solid (0.122 g, 48%). MS (ESI): 532.1 (M+H)⁺.

### Example 4

### Preparation of N-[2-[N-methyl-N-(2-methylpropyl)amino]thiazol-4-ylcarbonyl]-N′-[N-methyl-N-(4-pyridinylmethoxycarbonyl)-L-leucinyl]hydrazide

### a) N-(4-pyridinylmethoxycarbonyl)-L-leucine tert-butyl Ester

Following the procedure of Example 1(e)-1(f), except substituting L-leucine tert-butyl ester hydrochloride for L-leucine methyl ester hydrochloride in step (e), the title compound was prepared as a colorless oil (2.945 g, 64%). MS (ESI): 323.4 (M+H)⁺.

### b) N-Methyl-N-(4-pyridinylmethoxycarbonyl)-L-leucine Tertbutoxycarbonyl Ester

The compound of Example 3(a) (2.9 g, 8.99 mmol ) was dissolved in THF (40 mL) and methyl iodide (2.24 mL , 35.98 mmol) was added. The reaction mixture was cooled to 0° C. in a flask protected from moisture. Sodium hydride dispersion (1.214 mg, 13.49 mmol) was added cautiously and the suspension was stirred for 5 h at room temperature. Ethyl acetate was then added (to consume the sodium hydroxide formed from the excess of sodium hydride), followed by water, dropwise, to destroy the excess of sodium hydride. The solution was concentrated in vacuo, and the oily residue partitioned between ether and water. The ether layer was washed with saturated aqueous sodium bicarbonate. The product was extracted with ethyl acetate, the extract was washed with water, dried (MgSO₄), filtered and concentrated. The crude product was purified by column chromatography on silica gel (ethyl acetate/hexane, 3:1) to give a yellow oil (2.07 mg, 68%). MS (ESI): 337.5 (M+H)⁺.

### c) N-Methyl-N-(4-pyridinylmethoxycarbonyl)-L-leucine

To the compound of Example 3(b) (2.07 g, 6.15 mmol) in methylene chloride (20 mL) was added trifluoroacetic acid (3 mL). After stirring one hour at room temperature the solution was concentrated and the residue was redissolved in methylene chloride, washed with saturated aqueous sodium bicarbonate, dried (MgSO₄) and concentrated to afford the title compound as a white solid (1.72 g, 100%). MS (ESI): 281.3 (M+H)⁺.

### d) N-[2-[-methyl-N-(2-methylpropyl)amino]thiazol-4-ylcarbonyl]-N′-[N-methyl-N-(4-pyridinylmethoxycarbonyl)-L-leucinyl]hydrazide

Following the procedure of Example 1(a)-1(h), except substituting N-methyl isobutylamine for cis-2,6-dimethylmorpholine in step (a), and N-Methyl-N-(4-pyridinylmethoxycarbonyl)-L-leucine for N-(4-pyridinylmethoxycarbonyl)-L-leucine in step (h), the title compound was prepared as a pale yellow solid (91.8 mg; 43%). MS (ESI): 491.3 (M+H)⁺.

### Example 5

### Preparation of N-[2-(1-naphthyl)thiazol-4-ylcarbonyl]-N′-[N-(3-pyridinylmethoxycarbonyl)-L-leucinyl]hydrazide

### a) N-(3-pyridinylmethoxycarbonyl)-L-leucine

Following the procedure of Example 1(f)-1(g), except substituting 3-pyridylcarbinol for 4-pyridylcarbinol in step (f), the title compound was prepared as a white solid. MS (ESI): 267.2 (M+H)⁺.

### b) N-[2-(1-naphthyl)thiazol-4-ylcarbonyl]-N′-[N-(3-pyridinylmethoxycarbonyl)-L-leucinyl]hydrazide

Following the procedure of Examples 3(a)-3(c) and 3(e)-3(g), except substituting 1-naphthylboronic acid for 4-methyl-1-naphthalene boronic acid in step (e) and N-(3-pyridinylmethoxycarbonyl)-L-leucine for N-(4-pyridinylmethoxycarbonyl)-L-leucine in step (g), the title compound was prepared as a white solid (0.029 g, 28%). MS (ESI): 518.2 (M+H)⁺.

Example 6

### Preparation of N-[2-(1-naphthyl)thiazol-4-ylcarbonyl]-N′N-[N-2-pyridinylmethoxycarbonyl)-L-leucinyl]hydrazide

Following the procedure of Example 5(a)-5(b), except substituting 2-pyridylcarbinol for 3-pyridylcarbinol in step (a), the title compound was prepared as a white solid (0.084 g, 82%). MS (ESI): 518.2 (M+H)⁺.

### Example 7

### Preparation of N-[2-(5-acenaphthyl)thiazol-4-ylcarbonyl]-N′-[N-(4-pyridinylmethoxycarbonyl)-L-leucinyl]hydrazide

Following the procedure of Example 3(a)-3(g), except substituting 5-bromoacenaphthene for 1-bromo-4-methylnaphthalene in step (d), the title compound was prepared as a white solid (0.166 g, 74%). MS (ESI): 544.2 (M+H)⁺.

### Example 8

### Preparation of N-[2-[N-cyclopropylmethyl-N-(2-methylpropyl)amino]thiazol-4-ylcarbonyl]-N′-[N-methyl-N-(4-pyridinylmethoxycarbonyl)-L-leucinyl]hydrazide

Following the procedure of Example 2(a)-2(c), except substituting N-Methyl-N-(4pyridinylmethoxycarbonyl)-L-leucine for N-(4-pyridinylmethoxycarbonyl)-L-leucine in step (c), the title compound was prepared as a yellow solid (50 mg, 25%). MS (ESI): 531.3 (M+H)⁺.

### Example 9

### Preparation of N-[2-(N-cyclopropyl-N-cyclopropylmethylamino)thiazol-4-ylcarbonyl]-N′-[N-(4-pyridinylmethoxycarbonyl)-L-leucinyl]hydrazide

### a) N-cyclopropylmethyl Cyclopropylamine

Cyclopropylamine (1.14 g, 20.0 mmol, 1.4 mL) and cyclopropanecarboxaldehyde (1.40 g, 20.0 mmol, 1.5 mL) were dissolved in methylene chloride (10 mL) and stirred at room temperature. After two hours, the solution was dried (MgSO₄), and concentrated to afford the pure imine. The compound was dissolved in ether (10 mL), the solution was cooled to 0° C. and lithium aluminum hydride (30 mL, 30 mmol, 1 M in ether) was added slowly. The solution was stirred for two hours and then quenched at 0° C. with water (1.14 mL), 15% sodium hydroxyde (1.14 mL), water (3.42 mL). The solid was removed by filtration and washed with ether. The filtrate was dried (MgSO₄), filtered and concentrated to afford a colorless liquid (1.58 g, 71%). MS (ESI): 111.9 (M+H)⁺.

### b) N-[2-(N-cyclopropyl-N-cyclopropylmethylamino)thiazol-4-ylcarbonyl]-N′-[N-(4-pyridinylmethoxycarbonyl)-L-leucinyl]hydrazide

Following the procedure of Example 1(a)-1(h), except substituting N-cyclopropylmethyl cyclopropylamine for cis-2,6-dimethylmorpholine in step (a), the title compound was prepared as a white solid (165 mg, 88% yield). MS (ESI): 501.4 (M+H)⁺.

### Example 10

### Preparation of N-[2-[N-cyclopropylmethyl-N-(2-methylpropyl)amino]thiazol-4-ylcarbonyl]-N′-[N-(3-pyridinylmethoxycarbonyl)-L-leucinyl]hydrazide

Following the procedure of Example 2(a)-2(c), except substituting N-(3-pyridinylmethoxycarbonyl)-L-leucine for N-(4-pyridinylmethoxycarbonyl)-L-leucine in step (c), the title compound was prepared as a yellow solid (154 mg, 89%). MS (ESI): 517.4 (M+H)⁺.

### Example 11

### Preparation of N-[2-[N-cyclopropylmethyl-N-(2-methylpropyl)amino]thiazol-4-ylcarbonyl]-N′[N-(2-pyridinylmethoxycarbonyl)-L-leucinyl]hydrazide

Following the procedure of Example 2(a)-2(c), except substituting N-(2-pyridinylmethoxycarbonyl)-L-leucine for N-(4-pyridinylmethoxycarbonyl)-L-leucine in step (c), the title compound was prepared as a yellow solid (100 mg, 65%). MS (ESI): 517.3 (M+H)⁺.

### Example 12

### Preparation of N-[2-[N-cyclopropylmethyl-N-(2-methylpropyl)amino]thiazol-4-ylcarbonyl]-N′-[N-methyl-N-(3-p2pyridinylmethoxycarbonyl)-L-leucinyl]hydrazide

Following the procedure of Example 4(a)-4(d), except substituting 3-pyridylcarbinol for 4-pyridylcarbinol in step (a) and N-cyclopropylmethyl isobutylamine for N-methyl isobutylamine in step (d), the title compound was prepared as a yellow solid (30 mg, 22%). MS (ESI): 531.4 (M+H)⁺.

### Example 13

### Preparation of N-[2-(N-cyclopropyl-N-cyclopropylmethylamino)thiazol-4-ylcarbonyl]-N′-[N-(3-pyridinylmethoxycarbonyl)-L-leucinyl]hydrazide

Following the procedure of Example 1(a)-1(h), except substituting N-cyclopropylmethyl cyclopropylamine for cis-2,6-dimethylmorpholine in step (a) and 3-pyridylcarbinol for 4-pyridylcarbinol in step (f), the title compound was prepared as a white solid (85 mg, 43%). MS (ESI): 501.4 (M+H)⁺.

### Example 14

### Preparation of N-[2-(N-cyclopropyl-N-cyclopropylmethylamino)thiazol-4-ylcarbonyl]-N′-[N-methyl-N-(4-pyridinylmethoxycarbonyl)-L-leucinyl]hydrazide

Following the procedure of Example 4(a)-4(d), except substituting N-cyclopropylmethyl cyclopropylamine for N-methyl isobutylamine in step (d), the title compound was prepared as a white solid (58 mg, 35%). MS (ESI): 515.3 (M+H)⁺.

### Example 15

### Preparation of N-[2-[N,N-bis-(2-methylpropyl)amino]thiazol-4-ylcarbonyl]-N′-[N-(2-pyridinylmethoxycarbonyl)-L-leucinyl]hydrazide

Following the procedure of Example 1(a)-1 (h), except substituting diisobutylamine for cis-2,6-dimethylmorpholine in step (a) and 2-pyridylcarbinol for 4-pyridylcarbinol in step (f), the title compound was prepared as a yellow solid (140 mg, 77%). MS (ESI): 519.4 (M+H)⁺.

### Example 16

### Preparation of N-[N-(4-pyridinylmethoxycarbonyl)-L-leucinyl]-N′-[2-[1-(1,2,3,4-tetrahydroquinolino]thiazol-4-ylcarbonyl]hydrazide

Following the procedure of Example 1(a)-1(h), except substituting 1,2,3,4-tetrahydroquinoline for cis-2,6-dimethylmorpholine in step (a), the title compound was prepared as a yellow solid (168 mg, 88%). MS (ESI): 523.4 (M+H)⁺.

### Example 17

### Preparation of N-[4-methyl-2-(3-phenoxy)phenylpentanoyl]-N′-[2-(1-naphthyl)thiazol-4-ylcarbonyl]hydrazide

### a) 2-(3-phenoxyphenyl)-4-methylpent-4-enoic Acid

To a stirring solution of diisopropylamine (4.99 g, 49.3 mmol) in THF (50 mL) cooled to −78° C. was added n-butyllithium (19.4 mL, 48.5 mmol, 2.5M in hexane) dropwise. After stirring for 15 min at −78° C., a solution of 3-phenoxyphenylacetic acid (5.0 g, 21.9 mmol) in THF (20 mL) was added dropwise. The mixture was warmed to 0° C. then cooled to −78° C. and 3-bromo-2-methylpropene (4.4 g, 32.9 mmol) was added to the mixture in one portion. After stirring at −78° C. for 2 h, the reaction was quenched with 10 mL of water then concentrated. The residue was redissolved in water and extracted with ether (200 mL). The aqueous layer was acidified (3 N HCl) and extracted with ether (2×200 mL). The organic layers were combined, dried (MgSO₄), filtered and concentrated to yield the title compound as a white solid (5.4 g, 87%). 1H NMR (400 MHz, CDCl₃) d 7.36 (m, 3H), 7.14 (m, 2H), 7.01 (m, 4H), 4.78 (d, 2H), 3.82 (t, 1H), 2.83 (dd, 1H), 2.47 (dd, 1H), 1.75 (s, 3H).

### b) 2-(3-phenoxyphenyl)-4-methylpentanoic Acid

To a stirring solution of the compound of Example 17(a) (5.4 g, 19.1 mmol) in ethyl acetate (75 mL) was added palladium on carbon (2.0 g). After stirring under a balloon of hydrogen for 16 h, the mixture was filtered through celite. The filtrate was concentrated and the residue was purified by column chromatography (silica gel, ethyl acetate/hexane) to yield the title compound as a white solid (2.1 g, 39%). MS (ESI): 283.2 (M−H)⁻.

### c) (±)-N-[4-methyl-2-(3-phenoxy)phenylpentanoyl]-N′-[2-(1-naphthyl)thiazol-4-ylcarbonyl]hydrazide

Following the procedure of Example 1(h), except substituting 2-(1-naphthyl)thiazol-4-ylcarbonylhydrazide for N-[2-(cis-2,6-dimethyl-4-morpholino)thiazol-4-ylcarbonyl]hydrazide and 2-(3-phenoxyphenyl)-4-methylpentanoic acid for N-(4-pyridinylmethoxycarbonyl)-L-leucine, the title compound was prepared as a white solid (0.246 g, 82%). MS (ESI): 536.2 (M+H)⁺.

### Example 18

### Preparation of N-[2-[N-methyl-N-(2-methylpropyl)amino]thiazol-4-ylcarbonyl]-′-[-4-methyl-2-(3-phenyl)phenylpent-4-enoyl]hydrazide

### a) 2-(3-phenylphenyl)-4-methylpent-4-enoic Acid

Following the procedure of Example 17(a), except substituting 3-biphenylacetic acid for 3-phenoxyacetic acid, the title compound was prepared as a white solid. MS (ESI): 265.3 (M−H)⁻.

### b) N-[2-[N-methyl-N-(2-methylpropyl)amino]thiazol-4-ylcarbonyl]-N′-[-4-methyl-2-(3-phenyl)phenylpent-4enoyl]hydrazide

Following the procedure of Example 1(a)-(1(h), except substituting N-methyl isobutylamine for cis-2,6-dimethylmorpholine in step (a), and 2-(3-phenylphenyl-4-methylpent-4-enoic acid for N-(4-pyridinylmethoxycarbonyl)-L-leucine in step (h), the title compound was prepared as a white solid. MS (ESI): 477.3 (M+H)⁺.

### Example 19

### Preparation of N-[2-[N,N-bis-(2-methylpropyl)amino]thiazol-4-ylcarbonyl]-N′-[N-methyl-N-(3-pyridinylmethoxycarbonyl)-L-leucinyl]hydrazide

Following the procedure of Example 1(a)-1(h), except substituting diisobutylamine for cis-2,6-dimethylmorpholine in step (a) and 3-pyridylcarbinol for 4-pyridylcarbinol in step (f), the title compound was prepared as a yellow solid (110 mg, 30%). MS (ESI): 519.4 (M+H)⁺.

### Example 20

### Preparation of N-[2-(N-cyclopropyl-N-cyclopropylmethylamino)thiazol-4-ylcarbonyl]-N′-[N-methyl-N-(3-pyridinylmethoxycarbonyl)-L-leucinyl]hydrazide

Following the procedure of Example 4(a)-4(d), except substituting 3-pyridylcarbinol for 4-pyridylcarbinol in step (a) and N-cyclopropylmethyl cyclopropylamine for N-methyl isobutylamine in step (d), the title compound was prepared as a yellow solid (33 mg, 25%). MS (ESI): 515.4 (M+H)⁺.

### Example 21

### Preparation of N-[2-(N-cyclopropylmethyl-N-propylamino)thiazol-4-ylcarbonyl]-N′-[N-(3-pyridinylmethoxycarbonyl)-L-leucinyl]hydrazide

Following the procedure of Example 1(a)-1(h), except substituting N-cyclopropyl propylamine for cis-2,6-dimethylmorpholine in step (a) and 3-pyridylcarbinol for 4-pyridylcarbinol in step (f), the title compound was prepared as a yellow solid (40 mg, 25%). MS (ESI): 503.3 (M+H)⁺.

### Example 22

### Preparation of N-[2-[N-methyl-N-(2-methoxypropylamino]thiazol-4-ylcarbonyl]-N′-[4-methyl-2-(3-phenyl)phenylpentanoyl]hydrazide

### a) 2-3-phenylphenyl)-4-methylpentanoic Acid

Following the procedure of Example 17(a)-17(b), except substituting 3-biphenylacetic acid for 3-phenoxyacetic acid, the title compound was prepared as a white solid. MS (ESI): 267.4 (M−H)⁻.

### b) N-[2-[N-methyl-N-(2-methylpropyl)amino]thiazol-4-ylcarbonyl]-N′-[4-methyl-2-(3-phenyl)phenylpentanoyl]hydrazide

Following the procedure of Example 1(a)-1(h), except substituting N-methyl isobutylamine for cis-2,6-dimethylmorpholine in step (a), and 2-(3-phenylphenyl)-4-methylpentanoic acid for N-(4-pyridinylmethoxycarbonyl)-L-leucine in step (h), the title compound was prepared as a white solid (185 mg, 88%). MS (ESI): 479.4 (M+H)⁺.

### Example 23

### Preparation of N-[N-(2-methylpropyl)-N-(3-phenylphenyl)carbamoyl]-N′-[2-(1-naphthyl)thiazol-4-ylcarbonyl]hydrazide

### a) 3-phenylaniline

To a stirring solution of 3-nitrobiphenyl (1.2 g, 6.0 mmol) in ethyl acetate (25 mL) was added 10% Palladium on carbon (500 mg, 40% w/w). After stirring under a balloon of hydrogen for 24 h, the mixture was filtered through Celite and concentrated to yield the title compound as a white solid (0.956 g, 94%). MS (ESI): 170.0 (M+H)⁺.

### b) N-(3-phenyl)phenyl Isobutylamine

Following the procedure of Example 2(a)-2(b), except substituting 3-phenylaniline for isobutylamine, and isobutyryl chloride for cyclopropane carbonyl chloride in step (a), the title compound was prepared as a brown oil (1.1 g, 90%). MS (ESI): 226.1 (M+H)⁺.

### c) N-[2-(1-naphthyl)thiazol-4-ylcarbonyl]-N′-[[N-isobutyl-N-(3-biphenyl)]amido]hydrazine

To a solution of phosgene (0.289 mL, 1.93M in toluene) was added a mixture of the compound of Example 23(b) (0.126 g, 0.558 mmol) and N-methylmorpholine (0.056 g, 0.55 8 mmol) in dichloromethane (3 mL) dropwise. After stirring for 20 min., 2-(1-naphthyl)thiazol-4-ylcarbonylhydrazide (0.150 g, 0.558 mmol) and N-methylmorpholine (0.056 g, 0.558 mmol) in dichloromethane (3 mL) was added followed by DMF (3 mL). After stirring at 50° C. for 16 h, the solution was diluted with ethyl acetate and washed successively with water, saturated aqueous sodium bicarbonate, and brine. The organic layer was, dried (MgSO₄), filtered and concentrated. The residue was purified by column chromatography (silica gel, ethyl acetate/hexane) to yield the title compound as a white solid (0.122 g, 42%). MS (ESI): 521.3 (M+H)⁺.

### Example 24

### Preparation of N-[4-methyl-2-(3-phenyl)phenylpentanoyl]-N′-[2-(1-naphthyl)thiazol-4-ylcarbonyl]hydrazide

Following the procedure of Examples 3(a)-3(c) and 3(e)-3(g), except substituting 1-naphthylboronic acid for 4-methyl-1-naphthalene boronic acid in step (e) and 2-(3-phenylphenyl)-4-methylpentanoic acid for N-(4-pyridinylmethoxycarbonyl)-L-leucine in step (g), the title compound was prepared as a white solid (0.119 g, 49%). MS (ESI): 520.3 (M+H)⁺.

### Example 25

### Preparation of N-[4-methyl-2-(3-phenyl)phenylpentanoyl]-N′-[2-[N-(2-methylpropyl)-N-phenylamino]thiazol-4-ylcarbonyl]hydrazide

Following the procedure of Example 2(a)-2(c), except substituting aniline for isobutylamine and isobutyryl chloride for cyclopropane carbonyl chloride in step (a), and 2-(3-phenylphenyl)-4-methylpentanoic acid for N-(4-pyridinylmethoxycarbonyl)-L-leucine in step (c), the title compound was prepared as a white solid (72 mg, 52%). MS (ESI): 541.3 (M+H)⁺.

### Example 26

### Preparation of N-[2-(2-methoxy-1-naphthyl)thiazol-4-ylcarbonyl]-N′-[N-(4-pyridinylmethoxycarbonyl)-L-leucinyl]hydrazide

Following the procedure of Example 3(a)-3(g), except substituting 1-bromo-2-methoxynaphthalene for 1-bromo-4-methylnaphthalene in step (d), the title compound was prepared as a white solid (0.194 g, 85%). MS (ESI): 548.3 (M+H)⁺.

### Example 27

Preparation of N-[2-(2-benzyloxyphenyl)thiazol-4-ylcarbonyl]-N′-[4-methyl-2-(3-phenyl)phenylpentanoyl]hydrazide

### a) 2-benzyloxybromobenzene

To a stirring solution of 2-bromophenol (10.0 g, 57.8 mmol), and benzyl bromide (9.9 g, 57.8 mmol) in acetone (150 mL) was added K₂CO₃ (12.0 g, 86.7 mmol). After stirring at reflux for 4 h, the mixture was partitioned between ethyl acetate and water. The organic layer was washed with brine, dried (MgSO₄), filtered and concentrated. The residue was purified by column chromatography (silica gel, ethyl acetate/hexane) to yield the title compound as a colorless oil (15.2 g, 57.8 mmol). ¹HNMR (400 MHz, CDCl₃) d 7.62 (m, 1H), 7.54 (m, 2H), 7.45 (m, 2H), 7.37 (m, 1H), 7.28 (m, 1H), 6.98 (m, 1H), 6.01 (m, 1H), 5.17 (s, 2H).

### b) 2-benzyloxyphenylboronic Acid

To a stirring solution of the compound of Example 27(a) (15.2 g, 57.8 mmol) in THF (100 mL) at −78° C. was added dropwise n-BuLi (23.1 mL, 2.5M in hexane, 57.8 mmol). The mixture stirred at −78° C. for 25 min when added via cannulation to a stirring solution of triisopropylborate (54.4 g, 289 mmol) in THF (100 mL) at −78° C. After warming to room temperature and stirring for 3 h, the mixture was poured into 3N HCl (100 mL) and extracted with ethyl acetate (3×200 mL). The organic layers were combined, washed successively with water and brine, dried (MgSO₄), filtered and concentrated. The residue was purified by column chromatography (silica gel, ethyl acetate/hexane) to yield the title compound as a pale yellow solid (6.9 g, 30.3 mmol). ¹HNMR (400 MHz, CDCl₃) d 7.90 (d, 1H), 7.42 (m, 6H), 7.07 (t, 1H), 7.02 (d, 1H), 6.05 (s, 2H), 5.16 (s, 2H).

### c) N-[2-(2-benzyloxyphenyl)thiazol-4-ylcarbonyl]-N′-[4-methyl-2-(3-phenyl)phenylpentanoyl]hydrazide

Following the procedure of Examples 3(a)-3(c) and 3(e)-3(g), except substituting 2-benzyloxyphenylboronic acid acid for 4-methyl-1-naphthalene boronic acid in step (e) and 2-(3-phenylphenyl)-4-methylpentanoic acid for N-(4-pyridinylmethoxycarbonyl)-L-leucine in step (g), the title compound was prepared as a white solid (0.194 g, 85%). MS (ESI): 576.3 (M+H)⁺.

### Example 28

### Preparation of N-[2-(2-benzyloxy-1-naphthyl)thiazol-4-ylcarbonyl]-N′-[N-(4-pyridinylmethoxycarbonyl)-L-leucinyl]hydrazide

### a) 1-bromo-2-methoxymethoxynaphthalene

To a stirred suspension of sodium hydride (1.6 g, 40.3 mmol, 60% dispersion in mineral oil) in DMF (150 mL) at 0° C. was added 1-bromo-2-naphthol (5.0 g, 22.4 mmol) dropwise. After stirring for 20 min, bromomethyl methyl ether (2.8 g, 22.4 mmol) was added slowly. After warning to room temperature and stirring for 4 h, the mixture was poured into water and extracted with ethyl acetate. The organic phase was washed with saturated aqueous NaHCO₃ and brine then dried (MgSO₄), filtered and concentrated to a red oil (5.98 g, 100%). ¹H NMR (400 MHz, CDCl₃) d 8.27 (d, 1H), 7.79 (d, 2H), 7.60(t, 1H), 7.46 (m, 2H), 5.38 (s, 2H), 3.61 (s, 3H).

### b) ethyl 2-(2-methoxymethoxy-1-naphthyl)thiazole-4carboxylate

Following the procedure of Example 3(a)-3(e), except substituting 1-bromo-2-methoxymethoxynaphthalene for 1-bromo-4-methylnaphthalene in step (d), the title compound was prepared as an off-white solid (0.136 g, 15%). MS (ESI): 344.2 (M+H)⁺.

### c) ethyl 2-(2-hydroxy-1-naphthyl)thiazole-4-carboxylate

To a stirring solution of the compound of Example 28(b) (0.136 g, 0.397 mmol) in EtOH (3 mL) was added concentrated hydrochloric acid (5 drops). After stirring at reflux for 3 h, the solution was concentrated, redissolved in ethyl acetate, and washed successively with saturated aqueous NaHCO₃ and brine. The organic layer was dried (MgSO₄), filtered and concentrated. The residue was purified by column chromatography (silica gel, ethyl acetate/hexane) to yield the title compound as a white solid (0.080 g, 67%). MS (ESI): 300.2 (M+H)⁺.

### d) ethyl 2-(2-benzyloxy-1-naphthyl)thiazole-4-carboxylate

To a stirring solution of the compound of Example 28(c) (0.080 g, 0.268 mmol), benzyl alcohol (0.038 g, 0.348 mmol) and triphenylphosphine (0.091 g, 0.348 mmol) in THF (3 mL) at 0° C. was added diisopropyl azodicarboxylate (0.070 g, 0.348 mmol) dropwise. After stirring at room temperature for 16 h, the solution was concentrated and the residue purified by column chromatography (silica gel, ethyl acetate/hexane) to yield the title compound as a white solid (0.060 g, 58%). ¹H NMR (400 MHz, CDCl₃) d 8.41 (s, 1H), 8.12 (d, 1H), 7.91 (d, 1H), 7.80 (d, 1H), 7.52 (t, 1H), 7.41 (t, 1H), 7.34 (m, 6H), 5.24 (s, 2H), 4.49 (q, 2H), 1.44 (t, 3H).

### e) N-[2-(2-benzyloxy-1-naphthyl)thiazol-4-ylcarbonyl]-N′-[N-(4-pyridinylmethoxycarbonyl)-L-leucinyl]hydrazide

Following the procedure of Example 1(d)-1(h), except substituting ethyl 2-(2-benzyloxy-1-naphthyl)thiazole-4-carboxylate for ethyl 2-(cis-2,6-dimethyl-4-morpholino)thiazole-4-carboxylate (d), the title compound was prepared as a white solid (0.050 g, 52%). MS (ESI): 624.2 (M+H)⁺.

### Example 29

### Preparation of N-[2-[N,N-bis-(2-methylpropyl)amino]thiazol-4-ylcarbonyl]-N′-[N-methyl-N-(2-pyridinylmethoxycarbonyl)-L-leucinyl]hydrazide

Following the procedure of Example 4(a)-4(d), except substituting 2-pyridylcarbinol for 4-pyridylcarbinol in step (a) and diisobutylamine for N-methyl isobutylamine in step (d), the title compound was prepared as a yellow solid (40 mg, 20%). MS (ESI): 533.4 (M+H)⁺.

### Example 30

### Preparation of N-[2-(9-phenanthrenyl)thiazol-4-ylcarbonyl]-N′-[N-(4-pyridinylmethoxycarbonyl)-L-leucinyl]hydrazide

Following the procedure of Example 3(a)-3(g), except substituting 9-bromophenanthrene for 1-bromo-4-methylnaphthalene in step (d), the title compound was prepared as an off-white solid (0.085 g, 48%). MS (ESI): 568.2 (M+H)⁺.

### Example 31

### Preparation of N-[2-(9-anthracenyl)thiazol-4ylcarbonyl]-N′-[N-(4-pyridinylmethoxycarbonyl)-L-leucinyl]hydrazide

Following the procedure of Example 3(a)-3(g), except substituting 9-bromoanthracene for 1-bromo-4-methylnaphthalene in step (d), the title compound was prepared as a white solid (0.101 g, 67%). MS (ESI): 568.2 (M+H)⁺.

### Example 32

### Preparation of N-[2-[N,N-bis-(2-methylpropyl)amino]thiazol-4-ylcarbonyl]-N-(-tert-butoxycarbonyl-L-leucinyl)ydrazide

Following the procedure of Example 1(a)-1(d) and 1(h), except substituting diisobutylamine for cis-2,-dimethylmorpholine in step (a) and N-tert-butoxycarbonyl-L-leucine for N-(4-pyridinylmethoxycarbonyl)-L-leucine in step (h), the title compound was prepared as a yellow solid (950 mg, 78% yield). MS (ESI): 484.3 (M+H)⁺.

### Example 33

### Preparation of N-[2-[N,N-bis-(2-methylpropylamino]thiazol-4-ylcarbonyl]-N′-[N-(L-leucinyl)]hydrazide

Following the procedure of 4(c), except substituting N-[2-[N,N-bis-(2-methylpropyl)amino]thiazol-4-ylcarbonyl]-N′-(-tert-butoxycarbonyl-L-leucinyl)ydrazide for N-Methyl-N-(4-pyridinylmethoxycarbonyl)-L-leucine tertbutoxycarbonyl ester, the title compound was prepared as a yellow solid (370 mg, 85%). MS (ESI): 384.3 (M+H)⁺.

### Example 34

### Preparation of N-[2-(1-naphthyl)thiazol-4-ylcarbonyl]-N′-[N-methyl-N-(3-pyridinylmethoxycarbonyl)-L-leucinyl]hydrazide

Following the procedure of Examples 3(a)-3(c) and 3(e)-3(g), except substituting 1-naphthylboronic acid for 4-methyl-1-naphthalene boronic acid in step (e) and N-methyl-N-(3-pyridinylmethoxycarbonyl)-L-leucine for N-(4-pyridinylmethoxycarbonyl)-L-leucine in step (g), the title compound was prepared as a white solid (0.100 g, 59%). MS (ESI): 532.2 (M+H)⁺.

### Example 35

### Preparation of N-[2-[N,N-bis-(2-methylpropyl)amino]thiazol-4-ylcarbonyl]-N′-(N-picolinoyl-L-leucinyl)hydrazide

Following the procedure of Example 1(h), except substituting N-[2-[N,N-bis-(2-methylpropyl)amino]thiazol-4-ylcarbonyl]-N′-[N-(L-leucinyl)]hydrazide for N-[2-(cis-2,-dimethyl-4-morpholino)thiazol-4-ylcarbonyl]hydrazide and picolinic acid for N-(4-pyridinylmethoxycarbonyl)-L-leucine, the title compound was prepared as a yellow solid (40 mg, 30%). MS (ESI): 489.3 (M+H)⁺.

### Example 36

### Preparation of N-[2-[N,N-bis-(2-methylproplylamino]thiazol-4-ylcarbonyl]-N′-[N-(2-pyrazinecarbonyl)-L-leucinyl]hydrazide

Following the procedure of Example 1 (h), except substituting N-[2-[N,N-bis-(2-methylpropyl)amino]thiazol-4-ylcarbonyl]-N′-[N-(L-leucinyl)]hydrazide for N-[2-(cis-2,6-dimethyl-4-morpholino)thiazol-4-ylcarbonyl]hydrazide and pyrazinecarboxylic acid for N-(4-pyridinylmethoxycarbonyl)-L-leucine, the title compound was prepared as a yellow solid (45 mg, 35%). MS (ESI): 490.3 (M+H)⁺.

### Example 37

### Preparation of N-[N,N-bis-(2-methylpropyl)carbamoyl]-N′-[2-[N-(2-methylpropyl)-N-phenylamino]thiazol-4-ylcarbonyl]hydrazide

### a) Nisobutylaniline

Following the procedure of Example 2(a)-2(d), except substituting aniline for isobutylamine and isobutyryl chloride for cyclopropane carbonyl chloride in step (a), the title compound was prepared as an orange liquid (2.11 g, 83% yield). MS (ESI): 172.2 (M+Na)⁺.

### b) N-[2-[N-(2-methylpropyl)-N-phenyl]thiazol-4-ylcarbonyl]hydrazide

Following the procedure of Example 1(a)-1(d), except substituting N-isobutylaniline for cis-2,6-dimethylmorpholine in step (a), the title compound was prepared as a white solid. MS (ESI): 291.3 (M+H)⁺.

### c) N-[N,N-bis-(2-methylpropyl)carbamoyl]-N′-[2-[N-(2-methylpropyl)-N-phenylamino]thiazol-4-ylcarbonyl]hydrazide

Following the procedure of Example 23(c), except substituting N-[2-[N-(2-methylpropyl)-N-phenyl]thiazol-4-ylcarbonyl]hydrazide for 2-(1-naphthyl)thiazol-4-ylcarbonylhydrazide and diisobutylamine for N-(3-phenyl)phenyl isobutylamine, the title compound was prepared as a white solid (25 mg, 25%). MS (ESI): 446.3 (M+H)⁺.

### Example 38

### Preparation of N-(2-phenylthiazol-4-ylcarbonyl)-N′-[N-(4-pyridinylmethoxycarbonyl)-L-leucinyl]hydrazide

Following the procedure of Examples 3(a)-3(c) and 3(e)-3(g), except substituting phenylboronic acid for 4-methyl-1-naphthalene boronic acid in step (e), the title compound was prepared as a white solid (0.077 g, 27%). MS (ESI): 468.2 (M+H)⁺.

### Example 39

### Preparation of N-[2-[2-(4-tert-butoxycarbonyl)benzyloxyphenyl]thiazol-4-ylcarbonyl]-N′-[N-(4-pyridinylmethoxycarbonyl)-L-leucinyl]hydrazide

### a) tert-butyl 4-bromomethylbenzoate

To a stirring solution of 4-bromomethylbenzoic acid (4.0 g, 18.6 mmol) in cyclohexane (37 mL), dichloromethane (19 mL) and THF (2 mL) was added a solution of tert-butyl-2,2,2-trichloroacetimidate (8.1 g, 37.2 mmol) in cyclohexane (12 mL) followed by a catalytic amount of boron trifluoride etherate. After stirring at room temperature for 18 h, NaHCO₃ (4 g) was added and the mixture filtered. The mixture was filtered through a short plug of silica gel and concentrated to yield the title compound as a colorless oil that solidifies on standing (3.6 g, 71%). ¹H NMR (400 MHz, CDCl₃) d 7.98 (d, 2H), 7.44 (d, 2H), 4.50 (s, 2H), 1.59 (s, 9H).

### b) ethyl 2-(2-hydroxyphenyl)thiazole-4-carboxylate

Following the procedure of Example 28(a)-28(c), except substituting 2-bromophenol for 1-bromo-2-naphthol in step (a), the title compound was prepared as a white solid (0.560 g, 53%). MS (ESI): 250.1 (M+H)⁺.

### c) ethyl 2-[2-(4-tert-butoxycarbonylbenzyloxy)phenyl]thiazole-4-carboxylate

To a stirring mixture of the compound of Example 39(b) (0.094 g, 0.379 mmol) and potassium carbonate (0.136 g, 0.985 mmol) in acetone (10 mL) was added the compound of Example 39(a) (0.133 g, 0.417 mmol). After stirring at reflux for 16 h, the mixture was partitioned between ethyl acetate and water. The organic layer was washed with brine, dried (MgSO₄), filtered and concentrated. The residue was purified by column chromatography (silica gel, ethyl acetate/hexane) to yield the title compound as a white solid (0.160 g, 96%). MS (ESI): 440.2 (M+H)⁺.

### d) N-[2-[2-(4-tert-butoxycarbonyl)benzyloxyphenyl]thiazol-4-ylcarbonyl]-N′-[N-(4-pyridinylmethoxycarbonyl)-L-leucinyl]hydrazide

Following the procedure of Example 1(d)-1(h), except substituting ethyl 2-[2-(4-tert-butoxycarbonylbenzyloxy)phenyl]thiazole-4-carboxylate for ethyl 2-(cis-2,6-dimethyl-4-morpholino)thiazole-4-carboxylate in step (d), the title compound was prepared as a white solid (0.124 g, 47%). MS (ESI): 674.2 (M+H)⁺.

### Example 40

### Preparation of N-[2-[2-(4-carboxybenzyloxy)phenyl]thiazol-4-ylcarbonyl]-N′-[N-(4-pyridinylmethoxycarbonyl)-L-leucinyl]hydrazide

Following the procedure of 4(c), except substituting N-[2-[2-(4-tert-butoxycarbonyl)benzyloxyphenyl]thiazol-4-ylcarbonyl]-N′-[N-(4-pyridinylmethoxycarbonyl)-L-leucinyl]hydrazide for N-Methyl-N-(4-pyridinylmethoxycarbonyl)-L-leucine tertbutoxycarbonyl ester, the title compound was prepared as a pale yellow solid (0.130 g, 100%). MS (ESI): 618.2 (M+H)⁺.

### Example 41

### Preparation of N-[N-(4-tert-butoxycarbonylbenzyloxycarbonyl)-L-leucinyl]-N′-[2-[N-(2-methylpropyl)-N-phenylamino]thiazol-4-ylcarbonyl]hydrazide

### a) N-isobutylaniline

Following the procedure of Example 2(a)-2(b), except substituting aniline for isobutylamine and isobutyryl chloride for cyclopropane carbonyl chloride in step (a), the title compound was prepared as an orange liquid (2.11 g, 83%). MS (ESI): 335.3 (M+Na)⁺.

### b) (4-tert-butoxycarbonyl)benzyl Alcohol

Water (5 mL) and potassium carbonate (710 mg, 5.15 mmol) were added to a solution of the compound of Example 39(a) (280 mg, 1.03 mmol) in dioxane (5 mL). The mixture was heated at reflux overnight, then the dioxane was removed under reduced pressure. Methylene chloride was added followed by treatment with dilute HCl until all solid had dissolved. The organic phase was separated, washed with aqueous sodium bicarbonate, dried (MgSO₄), filtered and concentrated to afford the title compound as a white solid (214 mg, 100%). ¹H NMR (400 MHz, CDCl₃) d 7.89 (d, 2H), 7.33 (d, 2H), 4.67 (s, 2H), 3.08 (s, 1H), 1.57 (s, 9H).

### c) N-[N-(4-tert-butoxycarbonylbenzyloxycarbonyl)-L-leucinyl]-N′-[2-[N-(2-methylpropyl)-N-phenylamino]thiazol-4-ylcarbonyl]hydrazide

Following the procedure of Example 1(a)-1(h), except substituting N-isobutylaniline for cis-2,6-dimethylmorpholine in step (a) and (4-tertbutoxycarbonyl)benzyl alcohol for 4-pyridylcarbinol in step (f), the title compound was prepared as a white solid (15 mg, 17%). MS (ESI): 638.2 (M+H)⁺.

### Example 42

### Preparation of N-[2-[N,N-bis-(2-methylpropyl)amino]thiazol-4-ylcarbonyl]-N′-[N-(4-tert-butoxycarbonylbenzyloxycarbonyl)-L-leucinyl]hydrazide

Following the procedure of Example 1(a)-1(h), except substituting diisobutylamine for cis-2,6-dimethylmorpholine in step (a) and (4-tertbutoxycarbonyl)benzyl alcohol for 4-pyridylcarbinol in step (f), the title compound was prepared as a white solid (35 mg, 16%). MS (ESI): 618.4 (M+H)⁺.

### Example 43

### Preparation of N-[N-(4-carboxybenzyloxycarbonyl)-L-leucinyl]-N′-[2-[N-(2-methylpropyl)-N-phenylamino]thiazol-4-ylcarbonyl]hydrazide

Following the procedure of 4(c), except substituting N-[N-(4-tert-butoxycarbonylbenzyloxycarbonyl)-L-leucinyl]-N′-[2-[N-(2-methylpropyl)-N-phenylamino]thiazol-4-ylcarbonyl]hydrazide for N-Methyl-N-(4-pyridinylmethoxycarbonyl)-L-leucine tertbutoxycarbonyl ester, the title compound was prepared as a yellow solid (10 mg, 85%). MS (ESI): 582.2 (M+H)⁺.

### Example 44

### Preparation of N-(N-benzyloxycarbonyl-L-leucinyl)-N′-[2-[2-(4-tert-butoxycarbonyl)benzyloxyphenyl]thiazol-4-ylcarbonyl]hydrazide

Following the procedure of Example 1(d) and 1(h), except substituting ethyl 2-[2-(4-tert-butoxycarbonylbenzyloxy)phenyl]thiazole-4-carboxylate for ethyl 2-(cis-2,6-dimethyl-4-morpholino)thiazole-4-carboxylate in step (d) and N-benzyloxycarbonyl-L-leucine for N-(4-pyridinylmethoxycarbonyl)-L-leucine in step (h), the title compound was prepared as a white solid (0.102 g, 68%). MS (ESI): 673.2 (M+H)⁺.

### Example 45

### Preparation of N-(N-benzyloxycarbonyl-L-leucinyl)-N′-[2-[2-(4-carboxybenzyloxy)phenyl]thiazol-4-ylcarbonyl]hydrazide

Following the procedure of 4(c), except substituting N-(N-benzyloxycarbonyl-L-leucinyl)-N′-[2-[2-(4-tert-butoxycarbonyl)benzyloxyphenyl]thiazol-4-ylcarbonyl]hydrazide for N-Methyl-N-(4-pyridinylmethoxycarbonyl)-L-leucine tertbutoxycarbonyl ester, the title compound was prepared as a pale yellow solid (0.103 mg, 100%). MS (ESI): 632.2 (M+H)⁺.

### Example 46

### Preparation of N-[N-(6-methyl-3-pyridinylmethoxycarbonyl)-L-leucinyl]-N′-[2-(1-naphthyl)thiazol-4-ylcarbonyl]hydrazide

### a) 6-methyl-3-pyridylcarbinol

Following the procedure of Example 2(b), except substituting methyl 6-methylnicotinate for N-cyclopropylmethyl isobutyramide, the title compound was prepared as a yellow oil (4.32 g, 83%). MS (ESI): 123.8 (M+H)⁺.

### b) N-[N-(6-methyl-3-pyridinylmethoxycarbonyl)-L-leucinyl]-N′-[2-(1-naphthyl)thiazol-4-ylcarbonyl]hydrazide

Following the procedure of Example 5(a)-5(b), except substituting 6-methyl-3-idylcarbinol for 3-pyridylcarbinol in step (a), the title compound was prepared as a white solid (0.155 g, 63%). MS (ESI): 532.2 (M+H)⁺.

### Example 47

### Preparation of N-(N-benzyloxycarbonyl-L-leucinyl)-N′-[2-(N-cyclopropyl-N-cyclopropylmethylamino)thiazol-4-ylcarbonyl]hydrazide

Following the procedure of Example 1(a)-1(d) and 1(h), except substituting N-cyclopropylmethyl cyclopropylamine for cis-2,6-dimethylmorpholine in step (a) and N-benzyloxycarbonyl-L-leucine for N-(4-pyridinylmethoxycarbonyl)-L-leucine in step (h), the title compound was prepared as a white solid (156 mg, 75%). MS (ESI): 500.3 (M+H)⁺.

### Example 48

### Preparation of N-[2-(N-cyclopropyl-N-cyclopropylmethylamino)thiazol-4-ylcarbonyl]-N′-[N-(2-pyridinylmethoxycarbonyl)-L-leucinyl]hydrazide

Following the procedure of Example 1(a)-1(h), except substituting N-cyclopropylmethyl cyclopropylamine for cis-2,6-dimethylmorpholine in step (a) and 2-pyridylcarbinol for 4-pyridylcarbinol in step (f), the title compound was prepared as a white solid (260 mg, 73%). MS (ESI): 501.1 (M+H)⁺.

### Example 49

### Preparation of N-[2-(N-cyclopropyl-N-cyclopropylmethylamino)thiazol-4-ylcarbonyl]-N′-[N-(6-methyl-3-pyridinylmethoxycarbonyl)-L-leucinyl]hydrazide

Following the procedure of Example 1(a)-1(h), except substituting N-cyclopropylmethyl cyclopropylamine for cis-2,6-dimethylmorpholine in step (a) and 6-methyl-3-pyridylcarbinol for 4-pyridylcarbinol in step (f), the title compound was prepared as a white solid (151 mg, 71%). MS (ESI): 515.3 (M+H)⁺.

### Example 50

### Preparation of N-(N-tert-butoxycarbonyl-L-leucinyl)-N′-[2-(N-cyclopropyl-N-cyclopropylmethylamino)thiazol-4-ylcarbonyl]hydrazide

Following the procedure of Example 1(a)-1(d) and 1(h), except substituting N-cyclopropylmethyl cyclopropylamine for cis-2,6-dimethylmorpholine in step (a) and N-tert-butoxycarbonyl-L-leucine for N-(4-pyridinylmethoxycarbonyl)-L-leucine in step (h), the title compound was prepared as a white solid (1.2 g, 72%). MS (ESI): 466.3 (M+H)⁺.

### Example 51

### Preparation of N-[2-(N-cyclopropyl-N-cyclopropylmethylamino)thiazol-4-ylcarbonyl]-N′[-N-methyl-N-(2-pyridinylmethoxycarbonyl)-L-leucinyl]hydrazide

Following the procedure of Example 4(a)-4(d), except substituting 2-pyridylcarbinol for 4-pyridylcarbinol in step (a) and cyclopropylmethyl cyclopropylamine for N-methyl isobutylamine in step (d), the title compound was prepared as a white solid (60 mg, 25%). MS (ESI): 515.3 (M+H)⁺.

### Example 52

### Preparation of N-[2-(2-benzyloxyphenyl)thiazol-4-ylcarbonyl]-N′-[N-(4-methyl-3-pyridinylmethoxycarbonyl)-L-leucinyl]hydrazide

### a) N-(6-methyl-3-pyridinylmethoxycarbonyl)-L-leucine

Following the procedure of Example 1(f)-1(g), except substituting 6-methyl-3-pyridylcarbinol for 4-pyridylcarbinol in step (f), the title compound was prepared as an off-white solid (5.8 g, 100%). ¹H NMR (400 MHz, CDCl₃) d 8.36 (s, 1H), 7.61 (d, 1H), 7.15 (d, 1H), 5.85 (d, 1H), 5.01 (s, 2H), 4.20 (m, 1H), 2.50 (s, 3H), 1.62 (m, 2H), 1.49 (m, 1H), 0.87 (t, 6H).

### b) N-[2-(2-benzyloxyphenyl)thiazol-4-ylcarbonyl]-N′-[N-(4-methyl-3-pyridinylmethoxycarbonyl)-L-leucinyl]hydrazide

Following the procedure of Examples 3(a)-3(c) and 3(e)-3(g), except substituting 2-benzyloxyphenylboronic acid acid for 4-methyl-1-naphthalene boronic acid in step (e) and N-(6-methyl-3-pyridinylmethoxycarbonyl-L-leucine for N-(4-pyridinylmethoxycarbonyl)-L-leucine in step (g), the title compound was prepared as a white solid (178 mg, 99%). MS (ESI): 588.3 (M+H)⁺.

### Example 53

### Preparation of N-[2-(2-benzyloxyphenyl)thiazol-4-ylcarbonyl]-N′-[N-(2-methyl-3-pyridinylmethoxycarbonyl)-L-leucinyl]hydrazide

### a) 2-methyl-3-pyridylcarbinol

Following the procedure of Example 2(b), except substituting methyl 2-methylnicotinate for N-cyclopropylmethyl isobutyramide, the title compound was prepared as a pale yellow oil (4.89 g, 100%). MS (ESI): 123.8 (M+H)⁺.

### b) N-3-(6-methyl)pyridylmethoxycarbonyl-(L)-leucine

Following the procedure of Example 1(f)-1(g), except substituting 2-methyl-3-pyridylcarbinol for 4-pyridylcarbinol in step (f), the title compound was prepared as a white solid (6.73 g, 100%). MS (ESI): 281.3 (M+H)⁺. c) N-[2-(2-benzyloxyphenyl)thiazol-4-ylcarbonyl]-N′-[N-(2-methyl-3-pyridinylmethoxycarbonyl)-L-leucinyl]hydrazide

Following the procedure of Examples 3(a)-3(c) and 3(e)-3(g), except substituting 2-benzyloxyphenylboronic acid acid for 4-methyl-1-naphthalene boronic acid in step (e) and N-(2-methyl-3-pyridinylmethoxycarbonyl-L-leucine for N-(4-pyridinylmethoxycarbonyl)-L-leucine in step (g), the title compound was prepared as a pale yellow solid (179.1 mg, 99%). MS (ESI): 588.3 (M+H)⁺.

### Example 54

### Preparation of N-[2-[N-methyl-N-(2-methylpropylamino]thiazol-4-ylcarbonyl]-N′-[N-(6-methyl-3-pyridinylmethoxycarbonyl)-L-leucinyl]hydrazide

Following the procedure of Example 1(a)-1(h), except substituting N-methyl isobutylamine for cis-2,6-dimethylmorpholine in step (a) and 6-methyl-3-pyridylcarbinol for 4-pyridylcarbinol in step (f), the title compound was prepared as a pale yellow solid (215 mg, 100%). MS (ESI): 491.3 (M+H)⁺.

### Example 55

### Preparation of N-[2-[N-methyl-N-(2-methylpropyl)amino]thiazol-4-ylcarbonyl]-N′-[N-(2-methyl-3-pyridinylmethoxycarbonyl)-L-leucinyl]hydrazide

Following the procedure of Example 1(a)-1(h), except substituting N-methyl isobutylamine for cis-2,6-dimethylmorpholine in step (a) and 2-methyl-3-pyridylcarbinol for 4-pyridylcarbinol in step (f), the title compound was prepared as a pale yellow solid (215 mg, 100%). MS (ESI): 491.3 (M+H)⁺.

### Example 56

### Preparation of N-[2-(N-cyclopropyl-N-cyclopropylmethylamino)thiazol-4-ylcarbonyl]-N′(N-picolinoyl-L-leucinyl)hydrazide

### a) N-[2-(N-cyclopropyl-N-cyclopropylmethylamino)]thiazol-4-yl-carbonyl]-N′-(L-leucinyl)hydrazide

Following the procedure of 4(c), except substituting N-(N-tert-butoxycarbonyl-L-leucinyl)-N′-[2-(N-cyclopropyl-N-cyclopropylmethylamino)thiazol-4-ylcarbonyl]hydrazide for N-Methyl-N-(4-pyridinylmethoxycarbonyl)-L-leucine tertbutoxycarbonyl ester, the title compound was prepared as a white solid (668 mg, 81%). MS (ESI): 366.3 (M+H)⁺.

### b) N-[2-(N-cyclopropyl-N-cyclopropylmethylamino)thiazol-4-ylcarbonyl]-N′-(N-picolinoyl-L-leucinyl)hydrazide

Following the procedure of Example 1(h), except substituting N-[2-(N-cyclopropyl-N-cyclopropylmethylamino)]thiazol-4-yl-carbonyl]-N′-(L-leucinyl)hydrazide for N-[2-(cis-2,6-dimethyl-4-morpholino)thiazol-4-ylcarbonyl]hydrazide and picolinic acid for N-(4-pyridinylmethoxycarbonyl)-L-leucine, the title compound was prepared as a white solid (183 mg, 95%). MS (ESI): 471.2 (M+H)⁺.

### Example 57

### Preparation of N-[2-(N-cyclopropyl-N-cyclopropylmethylamino)thiazol-4-ylcarbonyl]-N′-[N-(2-methyl-3-pyridinylmethoxycarbonyl)-L-leucinyl]hydrazide

Following the procedure of Example 1(a)-1(h), except substituting N-cyclopropylmethyl cyclopropylamine for cis-2,6-dimethylmorpholine in step (a) and 2-methyl-3-pyridylcarbinol for 4-pyridylcarbinol in step (f), the title compound was prepared as a white solid (310 mg, 84%). MS (ESI): 515.4 (M+H)⁺.

### Example 58

### Preparation of N-[N-(3-tert-butoxycarbonylbenzyloxycarbonyl)-L-leucinyl]-N′-[2-(N-cyclopropyl-N-cyclopropylmethylamino)thiazol-4-ylcarbonyl]hydrazide

### a) 3-bromomethylbenzoic Acid

A mixture of 3-toluic acid (15.0 g, 110 mmol), N-bromosuccinimide (19.60 g, 110 mmol) and t-butyl peroxybenzoate (2.1 mL, 110 mmol) in carbon tetrachloride (50 mL) was heated at reflux overnight. The mixture was cooled and concentrated under reduced pressure. The residue obtained was washed with carbon tetrachloride and filtered under vacuum. The filtrate was evaporated to dryness to yield a white solid (12.57 g, 53%). ¹H NMR (400 MHz, CDCl₃) d 7.93 (m, 2H), 7.43 (m, 2H), 4.55 (s, 2H).

### b) tert-butyl 3-bromomethylbenzoate

Followiong the procedure of Exmaple 39(a), except substituting 3-bromomethylbenzoic acid for 4-bromomethylbenzoic acid, the title compound was prepared as a yellow oil (7.9 g, 100%). ¹H NMR (400 MHz, CDCl₃) d 7.93 (m, 2H), 7.43 (m, 2H), 4.55 (s, 2H), 1.55 (s, 9H).

### c) (3-tert-butoxycarbonyl)benzyl alcohol

Followiong the procedure of Example 41(b), except substituting tert-butyl 3-bromomethylbenzoate for tert-butyl 4-bromomethylbenzoate, the title compound was prepared as a yellow oil (5.6 g, 92%). MS (ESI): 208.1 (M+H)⁺.

### d) N-[N-(3-tert-butoxycarbonylbenzyloxycarbonyl)-L-leucinyl]-N′-[2-(N-cyclopropyl-N-cyclopropylmethylamino)thiazol-4-ylcarbonyl]hydrazide

Following the procedure of Example 1(a)-1(h), except substituting N-cyclopropylmethyl cyclopropylamine for cis-2,6-dimethylmorpholine in step (a) and (3-tertbutoxycarbonyl)benzyl alcohol for 4-pyridylcarbinol in step (f), the title compound was prepared as a white solid (90 mg, 29%). MS (ESI): 600.4 (M+H)⁺.

### Example 59

### Preparation of N-[2-(1-naphthyl)thiazol-4-ylcarbonyl]-N′-[N-(8-quinolinoyl)-L-leucinyl]hydrazide

### a) N-[2-(1-naphthyl)thiazol-4-ylcarbonyl]hydrazide

Following the procedure of Example 3(a)-3(f), except substituting 1-naphthalene boronic acid for 4-methyl-1-naphthalene boronic acid in step (e), the title compound was prepared as a pale yellow solid. MS (ESI): 270.1 (M+H)⁺.

### b) N-(N-tert-butoxycarbonyl-L-leucinyl)-N′-[2-(1-naphthyl)thiazol-4-ylcarbonyl]hydrazide

Following the procedure of Example 1(h), except substituting N-[2-(1-naphthyl)thiazol-4-ylcarbonyl]hydrazide for N-[2-(cis-2,6-dimethyl-4-morpholino)thiazol-4-ylcarbonyl]hydrazide in and N-tert-butoxycarbonyl-L-leucine for N-(4-pyridinylmethoxycarbonyl)-L-leucine in step, the title compound was prepared as a white solid (2.2 g, 96%). MS (ESI): 483.2 (M+H)⁺.

### c) N-(L-leucinyl)-N′-[2-(1-naphthyl)thiazol-4-ylcarbonyl]hydrazide

Following the procedure of 4(c), except substituting N-(N-tert-butoxycarbonyl-L-leucinyl)-N′-[2-(1-naphthyl)thiazol-4-ylcarbonyl]hydrazide for N-Methyl-N-(4-pyridinylmethoxycarbonyl)-L-leucine tertbutoxycarbonyl ester, the title compound was prepared as an off-white solid (1.7 g, 97%). MS (ESI): 383.3 (M+H)⁺.

### d) N-[2-(1-naphthyl)thiazol-4-ylcarbonyl]-N′-[N-(8-quinolinoyl)-L-leucinyl]hydrazide

Following the procedure of Example 1(h), except substituting N-(L-leucinyl)-N′-[2-(1-naphthyl)thiazol-4-ylcarbonyl]hydrazide for N-[2-(cis-2,6-dimethyl-4-morpholino)thiazol-4-ylcarbonyl]hydrazide and 8-quinolinecarboxylic acid for N-(4-pyridinylmethoxycarbonyl)-L-leucine, the title compound was prepared as a white solid (118 mg, 84%). MS (ESI): 538.2 (M+H)⁺.

### Example 60

### Preparation of N-[N-(2-methyl-3-pyridinylmethoxycarbonyl)-L-leucinyl]-N′-[2-(1-naphthyl)thiazol-4-ylcarbonyl]hydrazide

Following the procedure of Example 5(a)-5(b), except substituting 2-methyl-3-idylcarbinol for 3-pyridylcarbinol in step (a), the title compound was prepared as a white solid (0.160 g, 65%). MS (ESI): 532.2 (M+H)⁺.

### Example 61

### Preparation of N-[2-(1-naphthyl)thiazol-4-ylcarbonyl]-N′-(N-picolinoyl-L-leucinyl)hydrazide

Following the procedure of Example 59(a)-59(c), except substituting picolinic acid for 8-quinolinecarboxylic acid in step (d), the title compound was prepared as a white solid (0.086 g, 54%). MS (ESI): 488.3 (M+H)⁺.

### Example 62

### Preparation of N-[N-(3-carboxybenzyloxycarbonyl)-L-leucinyl]-N′-[2-(N-cyclopropyl-N-cyclopropylmethylamino)thiazol-4-ylcarbonyl]hydrazide

Following the procedure of 4(c), except substituting N-[N-(3-tert-butoxycarbonylbenzyloxycarbonyl)-L-leucinyl]-N′-[2-(N-cyclopropyl-N-cyclopropylmethylamino)thiazol-4-ylcarbonyl]hydrazide for N-Methyl-N-(4-pyridinylmethoxycarbonyl)-L-leucine tertbutoxycarbonyl ester, the title compound was prepared as a white solid (21 mg, 93%). MS (ESI): 544.3 (M+H)⁺.

### Example 63

### Preparation of N-[2-(1-naphthyl)thiazol-4-ylcarbonyl]-N′-[N-(2-quinolinoyl)-L-leucinyl]hydrazide

Following the procedure of Example 59(a)-59(d), except substituting 2-quinolinecarboxylic acid for 8-quinolinecarboxylic acid in step (d), the title compound was prepared as a white solid (0.123 g, 80%). MS (ESI): 538.2 (M+H)⁺.

### Example 64

### Preparation of N-[2-(1-naphthyl)thiazol-4-ylcarbonyl]-N′-[N-(3-quinolinoyl)-L-leucinyl]hydrazide

Following the procedure of Example 59(a)-59(d), except substituting 3-quinolinecarboxylic acid for 8-quinolinecarboxylic acid in step (d), the title compound was prepared as a white solid (0.109 g, 77%). MS (ESI): 538.2 (M+H)⁺.

### Example 65

### Preparation of N-[2-(1-naphthyl)thiazol-4-ylcarbonyl]-N′-[N-(4-methylpiperidinecarbonyl-L-leucinyl]hydrazide

Following the procedure of Example 59(a)-59(d), except substituting 1-methylpiperidine-4-carboxylic acid for 8-quinolinecarboxylic acid in step (d), the title compound was prepared as a white solid (0.059 g, 45%). MS (ESI): 508.2 (M+H)⁺.

### Example 66

### Preparation of N-[2-(1-naphthyl)thiazol-4-ylcarbonyl]-N′-[N-(4-quinolinoyl)-L-leucinyl]hydrazide

Following the procedure of Example 59(a)-59(d), except substituting 4-quinolinecarboxylic acid for 8-quinolinecarboxylic acid in step (d), the title compound was prepared as a white solid (0.096 g, 68%). MS (ESI): 538.3 (M+H)⁺.

### Example 67

### Preparation of N-[2-(1-naphthyl)thiazol-4-ylcarbonyl]-N′-[N-(5-quinolinoyl)-L-leucinyl]hydrazide

Following the procedure of Example 59(a)-59(d), except substituting 5-quinolinecarboxylic acid for 8-quinolinecarboxylic acid in step (d), the title compound was prepared as a white solid (0.089 g, 63%). MS (ESI): 538.3 (M+H)⁺.

### Example 68

### Preparation of N-[2-(1-naphthyl)thiazol-4-ylcarbonyl]-N′-[N-(7-quinolinoyl)-L-leucinyl]hydrazide

Following the procedure of Example 59(a)-59(d), except substituting 7-quinolinecarboxylic acid for 8-quinolinecarboxylic acid in step (d), the title compound was prepared as a white solid (0.106 g, 75%). MS (ESI): 538.2 (M+H)⁺.

### Example 69

### Preparation of N-[2-(1-naphthyl)thiazol-4-ylcarbonyl]-N′-[N-(6-quinolinoyl)-L-leucinyl]hydrazide

Following the procedure of Example 59(a)-59(d), except substituting 6-quinolinecarboxylic acid for 8-quinolinecarboxylic acid in step (d), the title compound was prepared as a white solid (0.111 g, 79%). MS (ESI): 538.2 (M+H)⁺.

### Example 70

### Preparation of N-[N-(1-isoquinolinoyl)-L-leucinyl]-N′-[2-(1-naphthyl)thiazol-4-ylcarbonyl]hydrazide

Following the procedure of Example 59(a)-59(d), except substituting 1-isoquinolinecarboxylic acid for 8-quinolinecarboxylic acid in step (d), the title compound was prepared as a white solid (0.076 g, 54%). MS (ESI): 538.2 (M+H)⁺.

### Example 71

### Preparation of N-[N-(3-isoquinolinoyl)-L-leucinyl]-N′-[2-(1-naphthyl)thiazol-4-ylcarbonyl]hydrazide

Following the procedure of Example 59(a)-59(d), except substituting 3-isoquinolinecarboxylic acid for 8-quinolinecarboxylic acid in step (d), the title compound was prepared as a white solid (0.055 g, 39%). MS (ESI): 538.2 (M+H)⁺.

### Example 72

### Preparation of N-[N-(4-methylimidazol-5-ylcarbonyl)-L-leucinyl]-N′-[2-(1-naphthyl)thiazol-4-ylcarbonyl]hydrazide

### a) 4-methylimidazole-5-carboxylic Acid

Following the procedure of Example 1(g), except substituting ethyl 4-methylimidazole-5-carboxylate for N-(4-pyridinylmethoxycarbonyl)-L-leucine methyl ester, the title compound was prepared as a white solid (0.428 g, 52%). MS (ESI): 126.8 (M+H)⁺.

### b) N-[N-(4-methylimidazol-5-ylcarbonyl)-L-leucinyl]-N′-[2-(1-naphthyl)thiazol-4-ylcarbonyl]hydrazide

Following the procedure of Example 1(h), except substituting N-(L-leucinyl)-N′-[2-(1-naphthyl)thiazol-4-ylcarbonyl]hydrazide for N-[2-(cis-2,6-dimethyl-4-morpholino)thiazol-4-ylcarbonyl]hydrazide and 4-methylimidazole-5-carboxylic acid for N-(4-pyridinylmethoxycarbonyl)-L-leucine, the title compound was prepared as a white solid (0.108 g, 84%). MS (ESI): 491.3 (M+H)⁺.

### Example 73

### Preparation of N-(N-benzyl-L-prolinyl-L-leucinyl)-N′-[2-(1-naphthyl)thiazol-4-ylcarbonyl]hydrazide

Following the procedure of Example 59(a)-59(d), except substituting N-benzyl proline for 8-quinolinecarboxylic acid in step (d), the title compound was prepared as a white solid (0.075 g, 50%). MS (ESI): 570.2 (M+H)⁺.

### Example 74

### Preparation of N-[N-(1-benzyl-5-methylimidazol-4-ylcarbonyl)-L-leucinyl]-N′-[2-(1-naphthyl)thiazol-4-ylcarbonyl]hydrazide

Following the procedure of Example 72(a)-72(b), except substituting 1-benzyl-5-methylimidazole-4-carboxylic acid for 4-methylimidazole-5-carboxylic acid in step (a), the title compound was prepared as a white solid (0.115 g, 75%). MS (ESI): 581.1 (M+H)⁺.

### Example 75

### Preparation of N-[N-(3-methylisonicotinoyl)-L-leucinyl]-N′-[2-(1-naphthyl)thiazol-4-ylcarbonyl]hydrazide

### a) 4-cyano-2-methylpyridine

To neat picoline N-oxide (10.0 g, 91.7 mmol) at room temperature was added iodoethane (51.5 g, 330 mmol) dropwise. After standing for 24 h, the salt was filtered off and washed with ether. The solid was dissolved in ethanol/water (70 mL/30 mL) and potassium cyanide (11.0 g, 172 mmol) in water (31 mL) was added dropwise over 100 min at 48-50° C. After the addition was complete, the solution continued stirring at the same temperature for 30 min. The solution was then extracted with chloroform. The organic layer was washed with brine, dried (MgSO₄), filtered and concentrated. The residue was purified by column chromatography (silica gel, ethyl acetate/hexane) to yield the title compound as a pale yellow oily solid (3.9 g, 36%). MS (ESI): 118.8 (M+H)⁺.

### b) 2-methylpyridine-4-carboxylic Acid

The compound of Example 75(a) (0.300 g, 2.5 mmol) was dissolved in concentrated hydrochloric acid (3 mL). After stirring at reflux for 18 h, the solution was concentrated to yield the title compound as a white solid (0.342 g, 100%). MS (ESI): 137.8 (M+H)⁺.

### c) N-[N-(3-methylisonicotinoyl)-L-leucinyl]-N′-[2-(1-naphthyl)thiazol-4-ylcarbonyl]hydrazide

Following the procedure of Example 1(h), except substituting N-(L-leucinyl)-N′-[2-(1-naphthyl)thiazol-4-ylcarbonyl]hydrazide for N-[2-(cis-2,6-dimethyl-4-morpholino)thiazol-4-ylcarbonyl]hydrazide and 2-methylpyridine-4-carboxylic acid for N-(4-pyridinylmethoxycarbonyl)-L-leucine, the tide compound was prepared as a white solid (0.095 g, 72%). MS (ESI): 502.2 (M+H)⁺.

### Example 76

### Preparation of N-[2-cyclopropylamino)thiazol-4-ylcarbonyl]-N′-[N-(2-pyridinylmethoxycarbonyl)-L-leucinyl]hydrazide

Following the procedure of Example 1(a)-1(h), except substituting cyclopropylamine for cis-2,6-dimethylmorpholine in step (a) and 2-pyridylcarbinol for 4-pyridylcarbinol in step (f), the title compound was prepared as a white solid (140 mg, 50%). MS (ESI): 447.3 (M+H)⁺.

### Example 77

### Preparation of N-[N-(2-benzoxazolyl)-L-leucinyl]-N′-[2-(1-naphthyl)thiazol-4-ylcarbonyl]hydrazide

A solution of the compound of Example 59(c) (100 mg, 0.26 mmol), 2-chlorobenzoxazole (40.2 mg, 0.26 mmol, 0.03 mL) and triethylamine (26.5 mg, 0.26 mmol, 0.365 mL) in 1:1 THF/methanol (1 mL) was heated at 60° C. for 48 h. The solution was diluted with ethyl acetate, washed with saturated aqueous NaHCO₃, water (2×) and saturated brine, then dried (MgSO₄), filtered and concentrated. The residue was purified by flash chromatography on 4 g of 230-400 mesh silica gel, eluting with 1:1 ethyl acetate/hexanes, to give the title compound as a pale yellow solid (50.2 mg, 38%). MS (ESI): 500.2 (M+H)⁺.

### Example 78

### Preparation of N-(N-benzyloxycarbonyl-L-leucinyl)-N′-[2-[N,N-bis-(2-methylpropyl)amino]oxazol-4-ylcarbonyl]hydrazide

### a) N,N-diisobutylurea

A solution of diisobutylamine (4.5 g, 34.8 mmol, 6.08 mL) and chlorosulfonyl isocyanate (4.93 g, 34.8 mmol, 3.03 mL) in THF (200 mL) was allowed to stir at room temperature for 20 min, then water (10 mL) was added and the solution was allowed to stir at room temperature for 17 h. The solution was concentrated, the residue was redissolved in ethyl acetate, washed with water, saturated aqueous NaHCo₃ and saturated brine, then dried (MgSO₄), filtered and concentrated to give the title compound as a colorless oil which crystallized upon standing (6.0 g, 100%). MS (ESI): 173.3 (M+H)⁺.

### b) N-(N-benzyloxycarbonyl-L-leucinyl)-N′-[2-[N,N-bis-(2-methylpropyl)amino]oxazol-4-ylcarbonyl]hydrazide

Following the procedure of Examples 1(c)-1(d) and 1(h), except substituting N,N-diisobutylurea for cis-2,6-dimethyl-4-morpholinothiourea in step (c) and N-benzyloxycarbonyl-L-leucine for N-(4-pyridinylmethoxycarbonyl)-L-leucine in step (h), the title compound was prepared as a white solid (126 mg, 64%). MS (ESI): 502.3 (M+H)⁺.

### Example 79

### Preparation of N-[2-[N-cyclopropyl-N-(2-methylpropyl)amino]thiazol-4-ylcarbonyl]-N′-[N-(2-pyridinylmethoxycarbonyl)-L-leucinyl]hydrazide

### a) N-cyclopropyl Isobutylamine

Following the procedure of Example 9(a), except substituting isobutyraldehyde for cyclopropane carboxaldehyde, the title compound was prepared as a colorless oil (1.9 g, 58%). MS (ESI): 113.9 (M+H)⁺.

### b) N-[2-[N-cyclopropyl-N-(2-methylpropyl)amino]thiazol-4-ylcarbonyl]-N′-[N-(2-pyridinylmethoxycarbonyl)-L-leucinyl]hydrazide

Following the procedure of Example 1(a)-1(h), except substituting N-cyclopropyl isobutylamine for cis-2,6-dimethylmorpholine in step (a) and 2-pyridyocarbinol for 4-pyridylcarbinol in step (f), the title compound was prepared as a white solid (150 mg, 69% yield). MS (ESI): 503.2 (M+H)⁺.

### Example 80

### Preparation of N-[2-[N-cyclopropyl-N-(2-methylpropyl)amino]thiazol-4-ylcarbonyl]-N′-[N-methyl-N-(2-pyridinylmethoxycarbonyl)-L-leucinyl]hydrazide

Following the procedure of Example 4(a)-4(d), except substituting 2-pyridylcarbinol for 4-pyridylcarbinol in step (a) and N-cyclopropyl isobutylamine for N-methyl isobutylamine in step (d), the title compound was prepared as a white solid (85 mg, 32%). MS (ESI): 517.3 (M+H)⁺.

### Example 81

### Preparation of N-[2-(1-naphthyl)thiazol-4-ylcarbonyl]-N′-[N-(1-piperazinecarbonyl)-L-leucinyl]hydrazide

### a) N-[2-(1-naphthyl)thiazol-4-ylcarbonyl]-N′-[N-(4-tert-butoxycarbonyl-1-piperazinecarbonyl)-L-leucinyl]hydrazide

Following the procedure of Example 23(c), except substituting N-tert-butoxycarbonylpiperazine for N-(3-phenyl)phenyl isobutylamine, the title compound was prepared as a white solid (131 mg, 85%). MS (ESI): 595.2 (M+H)⁺.

### b) N-[2-(1-naphthyl)thiazol-4-ylcarbonyl]-N′-[N-(1-piperazinecarbonyl)-L-leucinyl]hydrazide

Following the procedure of 4(c), except substituting N-[2-(1-naphthyl)thiazol-4-ylcarbonyl]-N′-[N-(4-tert-butoxycarbonyl-1-piperazinecarbonyl)-L-leucinyl]hydrazide for N-Methyl-N-(4-pyridinylmethoxycarbonyl)-L-leucine tertbutoxycarbonyl ester, the title compound was prepared as a white solid (47 mg, 54%). MS (ESI): 495.2 (M+H)⁺.

### Example 82

### Preparation of N-[4-methyl-2-(4-phenoxy)phenylpentanoyl]-N′-[2-(1-naphthyl)thiazol-4-ylcarbonyl]hydrazide

Following the procedure of Example 17(a)-17(c), except substituting 4-phenoxyphenylacetic acid for 4-phenoxyphenylacetic acid in step (a), the title compound was prepared as a white solid (134 mg, 67%). MS (ESI): 536.2 (M+H)⁺.

### Example 83

### Preparation of N-[2-[N-bis-(cyclopropylmethyl)amino]thiazol-4-ylcarbonyl]-N′-[N-(2-pyridinylmethoxycarbonyl)-L-leucinyl]hydrazide

### a) bis-(cyclopropylmethyl)amine

Following the procedure of Example 9(a), except substituting aminomethylcyclporopane for cyclopropylamine, the title compound was prepared as a colorless liquid (2.5 g, 96%). MS (ESI): 125.8 (M+H)⁺.

### b) N-[2-[N-bis-(cyclopropylmethyl)amino]thiazol-4-ylcarbonyl]-N′-[N-(2-pyridinylmethoxycarbonyl)-L-leucinyl]hydrazide

Following the procedure of Example 1(a)-1(h), except bis-(cyclopropylmethyl)amine for cis-2,6-dimethylmorpholine in step (a) and 2-pyridyocarbinol for 4-pyridylcarbinol in step (f), the title compound was prepared as a yellow solid (115 mg, 30%). MS (ESI): 515.3 (M+H)⁺.

### Example 84

### Preparation of N-[2-(N-cyclopropyl-N-cyclopropylmethylamino)thiazol-4-ylcarbonyl]-N′-[N-(2-quinolinoyl)-L-leucinyl]hydrazide

Following the procedure of Example 56(a)-56(b), except substituting 2-quinolinecarboxylic acid for picolinic acid in step (b), the title compound was prepared as a white solid (125 mg, 59%). MS (ESI): 521.2 (M+H)⁺.

### Example 85

### Preparation of N-[N-(8-quinolinoyl)-L-leucinyl]-N′-[2-(8-quinolinyl)thiazol-4-ylcarbonyl]hydrazide

### a) N-[2-(8-quinolinyl)thiazol-4-ylcarbonyl]hydrazide

Following the procedure of Example 3(a)-3(f), except substituting 8-bromoquinoline for 1-bromo-4-methylnaphthalene in step (d), the title compound was prepared as a pale yellow solid (0.306 g, 100%). MS (ESI): 271.2 (M+H)⁺.

### b) N-[N-(8-quinolinoyl)-L-leucinyl]-N′-[2-(8-quinolinyl)thiazol-4-ylcarbonyl]hydrazide

Following the procedure of Example 59(b)-59(d), except substituting N-[2-(8-quinolinyl)thiazol-4-ylcarbonyl]hydrazide for N-[2-(1-naphthyl)thiazol-4-ylcarbonyl]hydrazide in step (b), the title compound was prepared as a white solid (0.111 g, 66%). MS (ESI): 539.2 (M+H)⁺.

### Example 86

### Preparation of N-(N-benzyloxycarbonyl-L-leucinyl)-N′-[2-(1-naphthyl)thiazol-4-ylcarbonyl]hydrazide

Following the procedure of Examples 3(a)-3(c) and 3(e)-3(g), except substituting 1-naphthylboronic acid for 4-methyl-1-naphthalene boronic acid in step (e) and N-benzyloxycarbonyl-L-leucine for N-(4-pyridinylmethoxycarbonyl)-L-leucine in step (g), the title compound was prepared as a white solid (0.145 g, 60%) MS(ESI): 517.3 (M+H)⁺.

### Example 87

### Preparation of N-[2-(N-cyclopropyl-N-cyclopropylmethylamino)thiazol-4-ylcarbonyl]-N′-[N-(3-quinolinoyl)-L-leucinyl]hydrazide

Following the procedure of Example 56(a)-56(b), except substituting 3-quinolinecarboxylic acid for picolinic acid in step (b), the title compound was prepared as a white solid (150 mg, 75%). MS (ESI): 521.2 (M+H)⁺.

### Example 88

### Preparation of N-[2-N-cyclppropyl-N-cyclopropylmethylamino)thiazol-4-ylcarbonyl]-N′-[N-(3-isoquinolinoyl)-L-leucinyl]hydrazide

Following the procedure of Example 56(a)-56(b), except substituting 3-isoquinolinecarboxylic acid for picolinic acid in step (b), the title compound was prepared as a white solid (187 mg, 82%). MS (ESI): 521.1 (M+H)⁺.

### Example 89

### Preparation of N-[2-(N-cyclopropyl-N-cyclopropylmethylamino)thiazol-4-ylcarbonyl]-N′-[N-(6-quinolinoyl)-L-leucinyl]hydrazide

Following the procedure of Example 56(a)-56(b), except substituting 6-quinolinecarboxylic acid for picolinic acid in step (b), the title compound was prepared as a white solid (155 mg, 59%). MS (ESI): 521.3 (M+H)⁺.

### Example 90

### Preparation of N-[2-[N-bis-(cyclopropylmethyl)amino]thiazol-4-ylcarbonyl]-N′-[N-(2-methyl-3-pyridinylmethoxycarbonyl)-L-leucinyl]hydrazide

Following the procedure of Example 83(a)-83(b), except substituting 2-methyl-3-pyridylcarbinol for 2-pyridylcarbinol in step (b), the title compound was prepared as a yellow solid (105 mg, 46%). MS (ESI): 529.3 (M+H)⁺.

### Example 91

### Preparation of N-(N-benzyloxycarbonyl-L-b-tert-butylalanyl)-N′-[2-(1-naphthyl)thiazol-4-ylcarbonyl]hydrazide

### a) N-benzyloxycarbonyl-L-b-tert-butylalanine

To a stirring solution of L-b-tert-butylalanine (1.0 g, 6.89 mmol) in water (2.1 mL) and 5 N NaOH (1.38 mL) at 0° C. was added benzyl chloroformate (1.3 g, 7.58 mmol) and 2 N NaOH (3.8 mL) in ten alternating portions, over 1.5 h. After the additions are complete the mixture was stirred for another 30 min. at room temperature. The pH is then taken to 10 and the mixture is extracted with ether (50 mL). The aqueous layer was acidified to pH 3 with 3 N HCl and extracted with ether (3×50 mL). The organic layers are combined, dried (MgSO₄), filtered and concentrated to yield the title compound as a colorless oil (1.59 g, 83%). MS(ESI): 278.2 (M+H)⁻.

### b) N-(N-benzyloxycarbonyl-L-b-tert-butylalanyl)-N′-[2-(1-naphthyl)thiazol-4-ylcarbonyl]hydrazide

Following the procedure of Examples 3(a)-3(c) and 3(e)-3(g), except substituting 1-naphthylboronic acid for 4-methyl-1-naphthalene boronic acid in step (e) and N-benzyloxycarbonyl-L-tert-butylalanine for N-(4-pyridinylmethoxycarbonyl)-L-leucine in step (g), the title compound was prepared as a white solid (0.214 g, 87%). MS (ESI): 531.3 (M+H)⁺.

### Example 92

### Preparation of N-(N-benzyloxycarbonyl-L-cyclopropylalanyl)-N′-[2-(1-naphthyl)thiazol-4-ylcarbonyl]hydrazide

### a) N-benzyloxycarbonyl-L-allylglycine

Following the procedure of Example 91(a), except substituting L-allylglycine for L-tert-butylalanine, the title compound was prepared.

### b) N-benzyloxycarbonyl-L-cyclopropylalanine Methyl Ester

Diazomethane (4.8 mmol in 18 ml Et₂O) was added to a solution of the compound of Example 92(a) (0.210 g, 0.48 mmol) in 1 ml Et₂O at room temperature and was stirred for 5 minutes. Then Pd(OAc)₂ (2 mg) was added and the reaction was stirred overnight, filtered through silica gel, concentrated in vacuo, and was used in the next reaction without further purification (205 mg, 95%). MS (ESI): 300.1 (M+Na)⁺.

### c) N-benzyloxycarbonyl-L-cyclopropylalanine

Following the procedure of Example 1(g) except substituting N-benzyloxycarbonyl-L-cyclopropylalanine methyl ester for N-(4-pyridinylmethoxycarbonyl)-L-leucine methyl ester, the title compound was prepared as a white solid (165 mg, 82%). MS (ESI): 264.2 (M+H)⁺.

### d) N-(N-benzyloxycarbonyl-L-cyclopropylalanyl)-N′-[2-(1-naphthyl)thiazol-4-ylcarbonyl]hydrazide

Following the procedure of Examples 3(a)-3(c) and 3(e)-3(g), except substituting 1-naphthylboronic acid for 4-methyl-1-naphthalene boronic acid in step (e) and N-benzyloxycarbonyl-(L)-tert-butylalanine for N-(4-pyridinylmethoxycarbonyl)-L-leucine in step (g), the title compound was prepared as a white solid (0.054 g, 67%). MS (ESI): 515.2 (M+H)⁺.

### Example 93

### Preparation of N-[2-(1-naphthyl)thiazol-4-ylcarbonyl]-N′-[N-[3-(2-pyridyl)phenylacetyl]-L-leucinyl]hydrazide

### a) methyl 3-9trifluoromethanesulfonyloxyphenylacetate

To an oven-dried flask under Argon atmosphere containing sodium hydride (2.54 g, 60% dispersion in mineral oil, 63.5 mmol) was added anhydrous pentane (20 mL). The slurry was stirred for 5 min, allowed to settle, most of the pentane was removed, and anhydrous THF (40 mL) was added. To this suspension was added a solution of methyl 3-hydroxyphenylacetate (9.99 g, 60.1 mmol) in anhydrous THF (20 mL) and the reaction was stirred at room temperature for 20 min. To this mixture was then added a solution of N-phenyltrifluoromethanesulfonimide (22.53 g, 63.1 mmol)) in anhydrous THF (40 mL) and the reaction was stirred at room temperature until TLC analysis indicated the complete consumption of starting material (1.5 h). The reaction was quenched by the addition of H₂O (10 mL), concentrated to one half original volume, then diluted with CHCl₃ (200 mL) and washed with H₂O. The aqueous layer was washed with fresh CHCl₃ (50 mL), the combined organic layers were washed with 10% Na₂CO₃, H₂O, and brine, then dried (MgSO₄), filtered and concentrated. Column chromatography of the residue (silica gel, 5:95 EtOAc: hexanes, then 10:90 EtOAc: hexanes) gave 17.47 g of the title compound. ¹H NMR (400 MHz, CDCl₃) d 7.42 (m, 1H), 7.31-7.19 (m, 3H), 3.72 (s, 3H), 3.68 (s, 2H).

### b) methyl 3-(2-pyridyl)phenylacetate

To a solution of the compound of Example 93(a) (6.86 g, 23.0 mmol) in anhydrous dioxane (100 mL) was added 2-pyridylstannane (8.89 g, 24.1 mmol), LiCl (2.94 g, 69.3 mmol), 2,6-di-tert-butyl-4-methylphenol (a few crystals), and Pd(PPh₃)₄ (632.1 mg, 0.55 mmol). The reaction was protected from light with foil and heated to reflux overnight. The reaction was allowed to cool to room temperature and concentrated. Column chromatography of the residue (silica gel, 1:3 EtOAc: hexanes, then 1:2 EtOAc: hexanes) gave 3.85 g of the title compound: MS (ESI): 228.1 (M+H)⁺.

### c) 3-(2-pyridyl)phenyl Acetic Acid

Following the procedure of Examples 1(g), except substituting methyl 3-(2-pyridyl)phenylacetate for N-(4-pyridinylmethoxycarbonyl)-L-leucine methyl ester, the title compound was prepared. MS (ESI): 214.3 (M+H)⁺.

### e) N-[2-(1-naphthyl)thiazol-4-ylcarbonyl]-N′-[N-[3-(2-pyridyl)phenylacetyl]-L-leucinyl]hydrazide

Following the procedure of Examples 3(a)-3(c) and 3(e)-3(g), except substituting 1-naphthylboronic acid for 4-methyl-1-naphthalene boronic acid in step (e) and 3-(2-pyridyl)phenylacetic acid for N-(4-pyridinylmethoxycarbonyl)-L-leucine in step (g), the title compound was prepared as a white solid (0.149 g, 79%). MS (ESI): 578.1 (M+H)⁺.

### Example 94

### Preparation of N-[2-[N-bis-(cyclopropylmethyl)amino]thiazol-4-ylcarbonyl]-N′-(N-picolinyl-L-leucinyl)hydrazide

### a) N-[2-[N-bis-(cyclopropylmethyl)amino]thiazol-4-ylcarbonyl]-N′-(N-tert-butoxycarbonyl-L-leucinyl)hydrazide

Following the procedure of Example 1(a)-1(d) and 1(h), except substituting bis-(cyclopropylmethyl)amine cyclopropylamine for cis-2,6-dimethylmorpholine in step (a) and N-tert-butoxycarbonyl-L-leucine for N-(4-pyridinylmethoxycarbonyl)-L-leucine in step (h), the title compound was prepared as an orange oil. MS (ESI): 480.3 (M+H)⁺.

### b) N-[2-[N-bis-(cyclopropylmethyl)amino]thiazol-4-ylcarbonyl]-N′-(N-picolinyl-L-leucinyl)hydrazide

Following the procedure of Example 56(a)-56(b), except substituting N-[2-[N-bis-(cyclopropylmethyl)amino]thiazol-4-ylcarbonyl]-N′-(N-tert-butoxycarbonyl-L-leucinyl)hydrazide for N-[2-(N-cyclopropyl-N-cyclopropylmethylamino)thiazol-4-ylcarbonyl]-N′-(N-tert-butoxycarbonyl-L-leucinyl)hydrazide in step (a), the title compound was prepared as a yellow solid (74 mg, 41%). MS (ESI): 485.3 (M+H)⁺.

### Example 95

### Preparation of N-(N-benzyloxycarbonyl-L-leucinyl)-N′-[2-[N-bis-(cyclopropylmethyl)amino]thiazol-4-ylcarbonyl]hydrazide

Following the procedure of Example 1(a)-1(d) and 1(h), except substituting bis-(cyclopropylmethyl)amine for cis-2,6-dimethylmorpholine in step (a) and N-bejnzyloxycarbonyl L-leucine for N-(4-pyridinylmethoxycarbonyl)-L-leucine in step (h), the title compound was prepared as as a yellow solid (140 mg, 69%). MS (ESI): 514.3 (M+H)⁺.

### Example 96

### Preparation of N-[2-(N-cyclopropyl-N-cyclopropylmethylamino)thiazol-4-ylcarbonyl]-N′-[N-(6-methylnicotinoyl)-L-leucinyl]hydrazide

### a) 6-methylnicotinic Acid

Following the procedure of Example 1(g), except substituting methyl-6-methylnicotinate for N-(4-pyridinylmethoxycarbonyl)-L-leucine methyl ester, the title compound was prepared as a white solid (506 mg, 100%). MS (ESI): 137.9 (M+H)⁺.

### b) N-[2-(N-cyclopropyl-N-cyclopropylmethylamino)thiazol-4-ylcarbonyl]-N′-[N-(4-methylnicotinoyl)-L-leucinyl]hydrazide

Following the procedure of Example 56(a)-56(b), except substituting 6-methylnicotinic acid for picolinic acid in step (b), the title compound was prepared as a white solid (150 mg, 41%). MS (ESI): 485.4 (M+H)⁺.

### Example 97

### Preparation of N-[2-(N-cyclopropyl-N-cyclopropylmethylamino)thiazol-4-ylcarbonyl]-N′-[N-(2-methylnicotinoyl)-L-leucinyl]hydrazide

### a) 2-methylnicotinic Acid

Following the procedure of Example 1(g), except substituting methyl-2-methylnicotinate for N-(4-pyridinylmethoxycarbonyl)-L-leucine methyl ester, the title compound was prepared as a white solid (1.6 g, 100%). MS (ESI): 138.2 (M+H)⁺.

### b) N-[2-(N-cyclopropyl-N-cyclopropylmethylamino)thiazol-4-ylcarbonyl]-N′-[N-(2-methylnicotinoyl)-L-leucinyl]hydrazide

Following the procedure of Example 56(a)-56(b), except substituting 2-methylnicotinic acid for picolinic acid in step (b), the title compound was prepared as a white solid (170 mg, 71%). MS (ESI): 485.3 (M+H)⁺.

### Example 98

### Preparation of N-[2-(N-cyclopropyl-N-cyclopropylmethylamino)thiazol-4-ylcarbonyl]-N′-[N-(3-methylisonicotinoyl)-L-]leucinyl]hydrazide

Following the procedure of Example 56(a)-56(b), except substituting 2-methylpyridine-4-carboxylic acid for picolinic acid in step (b), the title compound was prepared as a white solid (120 mg, 57%). MS (ESI): 485.2 (M+H)⁺.

### Example 99

### Preparation of N-[2-(N-cyclopropyl-N-cyclopropylmethylamino)thiazol-4-ylcarbonyl]-N′-[N-(8-quinolinoyl)-L-leucinyl]hydrazide

Following the procedure of Example 56(a)-56(b), except substituting 8-quinolinecarboxylic acid for picolinic acid in step (b), the title compound was prepared as a white solid (200 mg, 94%). MS (ESI): 521.2 (M+H)⁺.

### Example 100

### Preparation of N-[2-[N-bis-(cyclopropylmethyl)amino]thiazol-4-ylcarbonyl]-N′-[N-(8-quinolinoyl)-L-leucinyl]hydrazide

Following the procedure of Example 56(a)-56(b), except substituting N-[2-[N-bis-(cyclopropylmethyl)amino]thiazol-4-ylcarbonyl]-N′-(N-tert-butoxycarbonyl-L-leucinyl) hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 8-quinolinecarboxylic acid for picolinic acid in step (b), the title compound was prepared as a yellow solid (25 mg, 12%). MS (ESI): 535.3 (M+H)⁺.

### Example 101

### Preparation of N-[2-[N-bis-(cyclopropylmethyl)amino]thiazol-4-ylcarbonyl]-N′-[N-(3-isoquinolinoyl)-L-leucinyl]hydrazide

Following the procedure of Example 56(a)-56(b), except substituting N-[2-[N-bis-(cyclopropylmethyl)amino]thiazol-4-ylcarbonyl]-N′-(N-tert-butoxycarbonyl-L-leucinyl)hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 3-isoquinolinecarboxylic acid for picolinic acid in step (b), the title compound was prepared as a yellow solid (25 mg, 10%). MS (ESI): 535.3 (M+H)⁺.

Example 102

### Preparation of N-[2-[4-(2,2-dimethylaminoethoxy)-1-naphthyl]thiazol-4-ylcarbonyl]-N′-[N-(8-quinolinoyl)-L-leucinyl]hydrazide

### a) 4-bromo-1-naphthol

To a vigorously stirred suspension of 1,4-dibromonaphthalene (15.3 g, 53.7 mmol) in hexane/THF (300 mL) at −78° C. was added n-butyllithium (22.3 mL, 56.4 mmol, 2.5 M in hexane) dropwise. After stirring for an additional 30 min. at −78° C., bis(trimethylsilyl)peroxide (11 g, 61.8 mmol) [Taddei, M., Ricci, A., Synthesis, 1986, 633] was added slowly via syringe. After warming to room temperature and stirring an additional 3 h, the mixture was diluted with ethyl acetate and washed with 1 N HCl then brine. The organic layer was dried (MgSO₄), filtered and concentrated. The residue was purified by column chromatography (silica gel, ethyl acetate/hexane) to yield the title compound as an off-white solid (6.5 g, 54%). MS (ESI): 221.1 (M+H)⁻.

### b) 4-bromo-naphthyl tert-butyl Ether

Following the procedure of Example 39(a), except substituting 4-bromo-1-naphthol for 4-bromomethylbenzoic acid, the title compound was prepared as a colorless oil (2.34 g, 62%). ¹H NMR (400 MHz, CDCl₃) d 8.28 (d, 1H), 8.18 (d, 1H), 7.67 (d, 1H), 7.60 (t, 1H), 7.54 (t, 1H), 7.01 (d, 1H), 1.51 (s, 9H).

### c) ethyl 2-(4-tert-butoxy-1-naphthyl)thiazole-4-carboxylate

Following the procedure of Example 3(a)-3(e), except substituting 4-bromo-1-naphthyl tert-butyl ether for 1-bromo-4-methylnaphthalene in step (d), the title compound was prepared as a white solid (0.783 g, 67%). MS (ESI): 356.2 (M+H)⁺.

### d) ethyl 2-(4-hydroxy-1-naphthyl)thiazole-4-carboxylate

Following the procedure of 4(c), except substituting ethyl 2-(4-tert-butoxy-1-naphthyl)thiazole-4-carboxylate for N-Methyl-N-(4-pyridinylmethoxycarbonyl)-L-leucine tertbutoxycarbonyl ester, the title compound was prepared as a yellow solid (0.580 g, 88%). MS (ESI): 300.1 (M+H)⁺.

### e) ethyl 2-[4-(2-N,N-dimethylaminoethoxy)-1-naphthyl]thiazol-4-carboxylate

Following the procedure of Example 28(d), except substituting ethyl 2-(4-hydroxy-1-naphthyl)thiazole-4-carboxylate for ethyl 2-(2-hydroxy-1-naphthyl)thiazole-4-carboxylate and 2-(N,N-dimethylamino)ethanol for benzyl alcohol, the title compound was prepared as a white solid (0.097 g, 52%). MS (ESI): 371.3 (M+H)⁺.

### f) N-(8-quinolinoyl)-L-leucine Methyl Ester

Following the procedure of Example 1(h), except substituting L-leucine methyl ester hydrochloride for N-[2-(cis-2,6-dimethyl-4-morpholino)thiazol-4-ylcarbonyl]hydrazide and 8-quinoline carboxylic acid for N-(4-pyridylmethoxycarbonyl)-L-leucine, the title compound was prepared as a white solid (0.637 g, 41%). MS (ESI): 301.2 (M+H)⁺.

### g) N-(8-quinolinoyl)-L-leucine

Following the procedure of Example 1(g), except substituting N-(8-quinolinoyl)-L-leucine methyl ester for N-(4-pyridylmethoxycarbonyl)-L-leucine methyl ester, the title compound was prepared as a white solid (0.150 g, 25%). ¹H NMR (400 MHz, CDCl₃) d 8.89 (t, 1H), 8.78 (d, 1H), 8.21 (d, 1H), 7.90 (d, 1H), 7.57 (t, 1H), 7.43 (t, 1H),4.88 (m, 1H), 1.92 (m, 3H), 1.03 (m, 6H).

### h) N-[2-[4-(2,2-dimethylaminoethoxy)-1-naphthyl]thiazol-4-ylcarbonyl]hydrazide

Following the procedure of Example 1(d), except substituting ethyl 2-[4-(2-N,N-dimethylaminoethoxy)-1-naphthyl]thiazole-4-carboxylate for ethyl 2-(cis-2,6-dimethyl-4-morpholino)thiazole-4-carboxylate, the title compound was prepared as a yellow solid (0.091 g, 100%). MS (ESI): 357.2 (M+H)⁺.

### i) N-[2-[4-(2,2-dimethylaminoethoxy)-1-naphthyl]thiazol-4-ylcarbonyl]-N′-[N-(8-quinolinoyl)-L-leucinyl]hydrazide

Following the procedure of Example 1(h), except substituting N-[2-[4-(2,2-dimethylaminoethoxy)-1-naphthyl]thiazol-4-ylcarbonyl]hydrazide for N-[2-(cis-2,6-dimethyl-4-morpholino)thiazol-4-ylcarbonyl]hydrazide and N-(8-quinolinoyl)-L-leucine for N-(4-pyridylmethoxycarbonyl)-L-leucine, the title compound was prepared as a yellow solid (0.050 g, 31%). MS (ESI): 625.2 (M+H)⁺.

### Example 103

### Preparation of N-[2-(N-cyclopropyl 1-N-cyclopropylmethylamino)thiazol-4-ylcarbonyl]-N′-[N-(7-quinolinoyl)-L-leucinyl]hydrazide

Following the procedure of Example 56(a)-56(b), except substituting 7-quinolinecarboxylic acid for picolinic acid in step (b), the title compound was prepared as a white solid (100 mg, 50%). MS (ESI): 521.2 (M+H)⁺.

### Example 104

### Preparation of N-[2-[N-bis-(cyclopropylmethyl)amino]thiazol-4-ylcarbonyl]-N′-[N-(6-methylnicotinoyl)-L-leucinyl]hydrazide

Following the procedure of Example 56(a)-56(b), except substituting N-[2-[N-bis-(cyclopropylmethyl)amino]thiazol-4-ylcarbonyl]-N′-(N-tert-butoxycarbonyl-L-leucinyl)hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 6-methylnicotinic acid for picolinic acid in step (b), the title compound was prepared as a yellow solid (40 mg, 15%). MS (ESI): 499.3 (M+H)⁺.

### Example 105

### Preparation of N-[2-[N-bis-(cyclopropylmethyl)amino]thiazol-4-ylcarbonyl]-N′-(N-methyl-L-prolinyl-L-leucinyl)hydrazide

Following the procedure of Example 56(a)-56(b), except substituting N-methyl-L-proline for picolinic acid in step (b), the title compound was prepared as a white solid (62 mg, 48%). MS (ESI): 477.3 (M+H)⁺.

### Example 106

### Preparation of N-(N-benzyloxycarbonyl-L-norvalinyl)-N′-[2-(1-naphthyl)thiazol-4-ylcarbonyl]hydrazide

Following the procedure of Examples 3(a)-3(c) and 3(e)-3(g), except substituting 1-naphthylboronic acid for 4-methyl-1-naphthalene boronic acid in step (e) and N-benzyloxycarbonyl-L-norvaline for N-(4-pyridinylmethoxycarbonyl)-L-leucine in step (g), the title compound was prepared as a white solid (180 mg, 96%). MS (ESI): 503.2 (M+H)⁺.

### Example 107

### Preparation of N-(N-benzyloxycarbonyl-L-isoleucinyl)-N′-[2-(1-naphthyl)thiazol-4-ylcarbonyl]hydrazide

Following the procedure of Examples 3(a)-3(c) and 3(e)-3(g), except substituting 1-naphthylboronic acid for 4-methyl-1-naphthalene boronic acid in step (e) and N-benzyloxycarbonyl-L-isoleucine for N-(4-pyridinylmethoxycarbonyl)-L-leucine in step (g), the title compound was prepared as a white solid (167 mg, 87%). MS (ESI): 517.1 (M+H)⁺.

### Example 108

### Preparation of N-[N-(4-dimethylaminomethylbenzoyl)-L-leucinyl]-N′-[2-(1-naphthyl)thiazol-4-ylcarbonyl]hydrazide

### a) methyl 4(N,N-dimethylaminomethyl)benzoate

Methyl 4-(bromomethyl)benzoate (2.0 g, 8.73 mmol) was added to a saturated solution of dimethylamine in methanol. After stirring for 25 min, the solution was concentrated and the residue was partitioned between 1N NaOH and ethyl acetate. The organic layer was washed with saturated brine, dired (MgSO₄), filtered, and concentrated to provide the title compound as a colorless liquid (1.67 g, 99%). ¹H NMR (250 MHz, CDCl₃) d 8.00 (d, 2H), 7.39 (d, 2H), 3.91 (s, 3H), 3.47 (d, 2H), 2.25 (s, 6H).

### b) 4-(N,N-dimethylaminomethyl)benzoic Acid

Following the procedure of Example 1(g), except substituting methyl 4-(N,N-dimethylaminomethyl)benzoate for N-(4-pyridinylmethoxycarbonyl)-L-leucine methyl ester, the title compound was prepared as a white solid (1.6 g, 100%). ¹H NMR (400 MHz, CD₃OD) d 7.94 (d, 2H), 7.36 (d, 2H), 3.64 (s, 2H), 2.35 (s, 6H).

### c) N-[N-(4-dimethylaminomethylbenzoyl)-L-leucinyl]-N′-[2-(1-naphthyl)thiazol-4-ylcarbonyl]hydrazide

Following the procedure of Example 59(a)-59(d), except substituting 4-(N,N-dimethylaminomethyl)benzoic acid for 8-quinolinecarboxylic acid in step (d), the title compound was prepared as a white solid (87 mg, 61%). MS (ESI): 544.2 (M+H)⁺.

### Example 109

### Preparation of N-(N-benzyloxycarbonyl-L-norleucinyl)-N′-[2-(1-naphthyl)thiazol-4-ylcarbonyl]hydrazide

Following the procedure of Examples 3(a)-3(c) and 3(e)-3(g), except substituting 1-naphthylboronic acid for 4-methyl-1-naphthalene boronic acid in step (e) and N-benzyloxycarbonyl-L-norleucine for N-(4-pyridinylmethoxycarbonyl)-L-leucine in step (g), the title compound was prepared as a white solid (184 mg, 96%). MS (ESI): 517.1 (M+H)⁺.

### Example 110

### Preparation of N-[N-(4-dimethylaminomethylbenzyloxycarbonyl)-L-leucinyl]-N′-[2-(1-naphthyl)thiazol-4-ylcarbonyl]hydrazide

### a) 4-(N,N-dimethylamino)benzyl Alcohol

To a stirring solution of the compound of Example 108(a) (1.63 g, 8.4 mmol ) in 25 mL of ether, cooled to 0° C., was added dropwise a 1 M solution of lithium aluminum hydride (8.4 mmol, 8.4 mL). After 5 min, the reaction was quenched by the addition of water (0.33 mL), 15% aqueous NaOH (0.33 mL) and water (1.0 mL). The precipitate was removed by filtration, washed with ether 2 times and the filtrate was concentrated to provide the title compound as a colorless oil (1.36 g, 98%). ¹H NMR (250 MHz, CDCl₃) d 7.32 (d, 2H), 7.28 (d, 2H), 4.68 (s, 2H), 3.41 (s, 2H), 2.22 (s, 6H).

### b) N-[N-(4-dimethylaminomethylbenzyloxycarbonyl)-L-leucinyl]-N′-[2-(1-naphthyl)thiazol-4-ylcarbonyl]hydrazide

Following the procedure of Example 1(e)-1(h), except substituting 4-(N,N-dimethylamino)benzyl alcohol for 4-pyridylcarbinol in step (f) and N-[2-(1-naphthyl)thiazol-4-ylcarbonyl]hydrazide for N-[2-(cis-2,6-dimethyl-4-morpholino)thiazol-4-ylcarbonyl]hydrazide, the title compound was prepared as a white solid (186 mg, 87%). MS (ESI): 574.3 (M+H)⁺.

### Example 111

### Preparation of N-(N-benzyloxcarbonyl-L-norvalinyl)-N′-[2-(2-benzyloxyphenyl)thiazol-4-ylcarbonyl]hydrazide

Following the procedure of Example 27(a)-27(c), except substituting N-benzyloxycarbonyl-L-norvaline for 2-(3-phenylphenyl)-4-methylpentanoic acid in step (c), the title compound was prepared as a white solid. MS (ESI): 559.0 (M+H)⁺.

### Example 112

### Preparation of N-[2-(N-cyclopropyl-N-cyclopropylmethylamino)thiazol-4-ylcarbonyl]-N′-[N-(4-methylimidazol-5-ylcarbonyl)-L-leucinyl]hydrazide

Following the procedure of Example 56(a)-56(b), except substituting 4-methylimidazole-5-carboxylic acid for picolinic acid in step (b), the title compound was prepared as a white solid (100 mg, 65%). MS (ESI): 474.3 (M+H)⁺.

### Example 113

### Preparation of N-[N-[4-(4-morpholinomethyhlbenzoyl]-L-leucinyl]-N′-[2-(1-naphthyl)thiazol-4-ylcarbonyl]hydrazide

Following the procedure of Example 108(a)-108(c), except substituting morpholine for dimethylmaine in step (a), the title compound was prepared as a white solid (0.097 g, 51%). MS (ESI): 586.2 (M+H)⁺.

### Example 114

### Preparation of N-[N-(2-methylnicotinoyl)-L-leucinyl]-N′-[2-(1-naphthylithiazol-4-ylcarbonyl]hydrazide

Following the procedure of Example 59(a)-59(d), except substituting 2-methylnicotinic acid for 8-quinolinecarboxylic acid in step (d), the title compound was prepared as a white solid (0.103 g, 60%). MS (ESI): 502.2 (M+H)⁺.

### Example 115

### Preparation of N-[N-(6-methylnicotinoyl)-L-leucinyl]-N′-[2-(1-naphthyl)thiazol-4-ylcarbonyl]hydrazide

Following the procedure of Example 59(a)-59(d), except substituting 6-methylnicotinic acid for 8-quinolinecarboxylic acid in step (d), the title compound was prepared as a white solid (0.134 g, 79%). MS (ESI): 502.2 (M+H)⁺.

### Example 116

### Preparation of N-[N-(4-methylimidazol-5-ylcarbonyl)-L-allylglycinyl]-N′-[2-(1-naphthyl)thiazol-4-ylcarbonyl]hydrazide

### a) N-tert-butoxycarbonyl-L-allylglycine

To a stirring solution of L-allylglycine (6.28 g, 54.5 mmol) in dioxane/water/1 N NaOH (110 mL/55 mL/55 mL ) at 0° C. was added di-tert-butyl dicarbonate (12.5 g, 57.2 mmol). After stirring for 30 min., the solution was concentrated and redissolved in 60 mL of water. A layer of ethyl acetate was added and the aqueous layer was acidified to pH 3 with 0.3 N KHSO₄. The aqueous layer was extracted with ethyl acetate (2×). The organic layers were combined, washed with water (2×), dried (MgSO₄), filtered and concentrated to yield the title compound as a white solid (10.11 g, 86%). MS (ESI): 453.2 (2 M+Na)⁺.

### b) N-[N-(4-methylimidazol-5-ylcarbonyl)-L-allylglycinyl]-N′-[2-(1-naphthyl)thiazol-4-ylcarbonyl]hydrazide

Following the procedure of Example 59(a)-59(d), except substituting N-tert-butoxycarbonyl-L-allylglycine for N-tert-butoxycarbonyl-L-leucine in step (b) and 4-methylimidazole-5-carboxylic acid for 8-quinolinecarboxylic acid in step (d), the title compound was prepared as a white solid (0.112 g, 67%). MS (ESI): 475.1 (M+H)⁺.

### Example 117

### Preparation of N-(N-b-tert-butoxycarbonyl-L-tert-butylalanyl)-N′-[2-(N-cyclopropyl-N-cyclopropylmethylamino)thiazol-4-ylcarbonyl]hydrazide

### a) N-tert-butoxycarbonyl-L-b-tert-butylalanine

Following the procedure of Example 116(a), except substituting L-tert-butylalanine for L-allylglycine, the title compound was prepared as a white solid (2.36 g, 70%). MS(ESI): 268.3 (M+Na)⁺.

### b) N-(N-b-tert-butoxycarbonyl-L-tert-butylalanyl)-N′-[2-(N-cyclopropyl-N-cyclopropylmethylamino)thiazol-4-ylcarbonyl]hydrazide

Following the procedure of Example 1(a)-1(d) and 1(h), except substituting N-cyclopropylmethyl cyclopropylamine for cis-2,6-dimethylmorpholine in step (a) and N-tert-butoxycarbonyl-L-b-tert-butylalanine for N-(4-pyridinylmethoxycarbonyl)-L-leucine in step (h), the title compound was prepared as a white solid (0.96 g, 100%). MS (ESI): 480.3 (M+H)⁺.

### Example 118

### Preparation of N-[2-(N-cyclopropyl-N-cyclopropylmethylamino)thiazol-4-ylcarbonyl]-N′-[N-(8-quinolinoyl)-L-b-tert-butylalanyl]hydrazide

Following the procedure of Example 56(a)-56(b), except substituting N-(N-b-tert-butoxycarbonyl-L-tert-butylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 8-quinolinecarboxylic acid for picolinic acid in step (b), the title compound was prepared as a white solid (160 mg, 82%). MS (ESI): 535.3 (M+H)⁺.

### Example 119

### Preparation of N-[N-(4-methylimidazol-5-ylcarbonyl)-L-b-tert-butylalanyl]-N′-[2-(1-naphthyl)thiazol-4-ylcarbonyl]hydrazide

Following the procedure of Example 59(a)-59(d), except substituting N-tert-butoxycarbonyl-L-b-tert-butylalanine for N-tert-butoxycarbonyl-L-leucine in step (b) and 4-methylimidazole-5-carboxylic acid for 8-quinolinecarboxylic acid in step (d), the title compound was prepared as a white solid (0.096 g, 58%). MS (ESI): 505.2 (M+H)⁺.

### Example 120

### Preparation of N-[2-(N-cyclopropyl-N-cyclopropylmethylamino)thiazol-4-ylcarbonyl]-N′-[N-(4-methylimidazol-5-ylcarbonyl)-L-b-tert-butylalanyl]hydrazide

Following the procedure of Example 56(a)-56(b), except substituting N-(N-b-tert-butoxycarbonyl-L-tert-butylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 4-methylimidazole-5-carboxylic acid for picolinic acid in step (b), the title compound was prepared as a white solid (180 mg, 78%). MS (ESI): 488.2 (M+H)⁺.

### Example 121

### Preparation of N-[2-(1-naphthyl)thiazol-4-ylcarbonyl]-N′-(N-picolinoyl-L-b-tert-butylalanyl)hydrazide

Following the procedure of Example 59(a)-59(d), except substituting N-tert-butoxycarbonyl-L-b-tert-butylalanine for N-tert-butoxycarbonyl-L-leucine in step (b) and picolinic acid for 8-quinolinecarboxylic acid in step (d), the title compound was prepared as a white solid (0.098 g, 62%). MS (ESI): 502.3 (M+H)⁺.

### Example 122

### Preparation of N-[2-(1-naphthyl)thiazol-4-ylcarbonyl]-N′-[N-(8-quinolinoyl)-L-b-tert-butylalanyl]hydrazide

Following the procedure of Example 59(a)-59(d), except substituting N-tert-butoxycarbonyl-L-b-tert-butylalanine for N-tert-butoxycarbonyl-L-leucine in step (b), the title compound was prepared as a white solid (0.083 g, 46%). MS (ESI): 552.2 (M+H)⁺.

### Example 123

### Preparation of N-[2-(1-naphthyl)thiazol-4-ylcarbonyl]-N′-(N-picolinoyl-L-allylglycinyl)hydrazide

Following the procedure of Example 59(a)-59(d), except substituting N-tert-butoxycarbonyl-L-allylglycine for N-tert-butoxycarbonyl-L-leucine in step (b) and picolinic acid for 8-quinolinecarboxylic acid in step (d), the title compound was prepared as a white solid (0.141 g, 84%). MS (ESI): 472.2 (M+H)⁺.

### Example 124

### Preparation of N-[2-(1-naphthyl)thiazol-4-ylcarbonyl]-N′-(N-picolinoyl-L-b-cyclopropylalanyl)hydrazide

### a) N-tert-butoxycarbonyl-L-b-cyclopropylalanine Methyl Ester

To a stirring solution of the compound of Example 116(a) (7.81 g, 36.3 mmol) in ether (100 mL) at 0° C. was added a solution of diazomethane (made from 10 eq of 1-methyl-3-nitro-1-nitrosoguanidine in ether (500 mL) and 40% NaOH (500 mL) at 0° C.). After stirring for 10 min., Pd(OAc)₂ (0.300 g) was added to the solution. After 20 min., the solution was concentrated and the residue was filtered through a short plug of silica gel to remove unused catalyst. Concentration of the solution yielded the title compound as a golden yellow oil (8.29 g, 99%). ¹H NMR (400 MHz, CDCl₃) d 5.17 (d, 1H), 4.39 (m, 1H), 3.73 (s, 3H), 1.66 (t, 2H), 1.44 (s, 9H), 0.68 (m, 1H), 0.49 (m, 2H), 0.08 (m, 1H).

### b) N-tert-butoxycarbonyl-L-b-cyclopropylalanine

Following the procedure of Example 1(g), except substituting N-tert-butoxycarbonyl-L-b-cyclopropylalanine methyl ester for N-(4-pyridinylmethoxycarbonyl)-L-leucine methyl ester, the title compound was prepared as a golden yellow oil (6.37 g, 82%). MS (ESI): 252.3 (M+Na)⁺.

### c) N-[2-(1-naphthyl)thiazol-4-ylcarbonyl]-N′-(N-picolinoyl-L-cyclopropylalanyl)hydrazide

Following the procedure of Example 59(a)-59(d), except substituting N-tert-butoxycarbonyl-L-b-cyclopropylalanine for N-tert-butoxycarbonyl-L-leucine in step (b) and picolinic acid for 8-quinolinecarboxylic acid in step (d), the title compound was prepared as a white solid (0.114 g, 71%). MS (ESI): 486.1 (M+H)⁺.

### Example 125

### Preparation of N-[N-(6-methylnicotinoyl)-L-b-cyclopropylalanyl]-N′-[2-(1-naphthyl)thiazol-4-ylcarbonyl]hydrazide

Following the procedure of Example 59(a)-59(d), except substituting N-tert-butoxycarbonyl-L-b-cyclopropylalanine for N-tert-butoxycarbonyl-L-leucine in step (b) and 6-methylnicotinic acid for 8-quinolinecarboxylic acid in step (d), the title compound was prepared as a white solid (0.097 g, 59%). MS (ESI): 500.1 (M+H)⁺.

### Example 126

### Preparation of N-[N-(4-methylimidazol-5-ylcarbonyl)-L-b-cyclopropylalanyl]-N′-[2-(1-naphthyl)thiazol-4-ylcarbonyl]hydrazide

Following the procedure of Example 59(a)-59(d), except substituting N-tert-butoxycarbonyl-L-b-cyclopropylalanine for N-tert-butoxycarbonyl-L-leucine in step (b) and 4-methylimidazole-5-carboxylic acid for 8-quinolinecarboxylic acid in step (d), the title compound was prepared as a white solid (0.095 g, 59%). MS (ESI): 489.1 (M+H)⁺.

### Example 127

### Preparation of N-[2-(1-naphthyl)thiazol-4-ylcarbonyl]-N′-[N-(8-quinolinoyl)-L-b-cyclopropylalanyl]hydrazide

Following the procedure of Example 59(a)-59(d), except substituting N-tert-butoxycarbonyl-L-b-cyclopropylalanine for N-tert-butoxycarbonyl-L-leucine in step (b), the title compound was prepared as a white solid (0.138 g, 78%). MS (ESI): 536.2 (M+H)⁺.

### Example 128

### Preparation of N-[N-(6-methylnicotinoyl)-L-b-tert-butylalanyl]-N′-[2-(1-naphthyl)thiazol-4-ylcarbonyl]hydrazide

Following the procedure of Example 59(a)-59(d), except substituting N-tert-butoxycarbonyl-L-b-tert-butylalanine for N-tert-butoxycarbonyl-L-leucine in step (b) and 6-methylnicotinic acid for 8-quinolinecarboxylic acid in step (d), the title compound was prepared as a white solid (0.124 g, 73%). MS (ESI): 516.1 (M+H)⁺.

### Example 129

### Preparation of N-[2-(N-cyclopropyl)-N-cyclopropylmethylamino)thiazol-4-ylcarbonyl]-N′-(N-picolinoyl-L-b-tert-butylalanyl)hydrazide

Following the procedure of Example 56(a)-56(b), except substituting N-(N-b-tert-butoxycarbonyl-L-tert-butylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a), the title compound was prepared as a white solid (143 mg, 83%). MS (ESI): 485.1 (M+H)⁺.

### Example 130

### Preparation of N-[2-(N-cyclopropyl-N-cyclopropylmethylamino)thiazol-4-ylcarbonyl]-N′-[N-(3-isoquinolinoyl)-L-b-tert-butylalanyl]hydrazide

Following the procedure of Example 56(a)-56(b), except substituting N-(N-b-tert-butoxycarbonyl-L-tert-butylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 3-isoquinolinecarboxylic acid for picolinic acid in step (b), the title compound was prepared as a white solid (138 mg, 85%). MS (ESI): 535.1 (M+H)⁺.

### Example 131

### Preparation of N-(N-tert-butoxycarbonyl-L-b-cyclopropylalanyl)-N′-[2-(N-cyclopropyl-N-cyclopropylmethylamino)thiazol-4-ylcarbonyl]hydrazide

Following the procedure of Example 1(a)-1(d) and 1(h), except substituting N-cyclopropylmethyl cyclopropylamine for cis-2,6-dimethylmorpholine in step (a) and N-tert-butoxycarbonyl-L-b-cyclopropylalanine for N-(4-pyridinylmethoxycarbonyl)-L-leucine in step (h), the title compound was prepared as a white solid (1.375 g, 76%). MS (ESI): 464.2 (M+H)⁺.

### Example 132

### Preparation of N-[2-(N-cyclopropylmethyl-N-propylamino)thiazol-4-ylcarbonyl]-N′-[N-(6-methyl-3-pyridinylmethoxycarbonyl)-L-leucinyl]hydrazide

Following the procedure of Example 1(a)-1(h), except substituting N-cyclopropyl propylamine for cis-2,6-dimethylmorpholine in step (a) and 6-methyl-3-pyridylcarbinol for 4-pyridylcarbinol in step (f), the title compound was prepared as an orange solid (84 mg, 33%). MS (ESI): 517.3 (M+H)⁺.

### Example 133

### Preparation of N-[N-(6-methylnicotinoyl)-L-allylglycinyl]-N′-[2-(1-naphthyl)thiazol-4-ylcarbonyl]hydrazide

Following the procedure of Example 59(a)-59(d), except substituting N-tert-butoxycarbonyl-L-allylglycine for N-tert-butoxycarbonyl-L-leucine in step (b) and 6-methylnicotinic acid for 8-quinolinecarboxylic acid in step (d), the title compound was prepared prepared as a white solid (0.097 g, 66%). MS (ESI): 486.1 (M+H)⁺.

### Example 134

### Preparation of N-[2-(1-naphthyl)thiazol-4-ylcarbonyl]-N′-[N-(8-quinolinoyl)-L-allylglycinyl]hydrazide

Following the procedure of Example 59(a)-59(d), except substituting N-tert-butoxycarbonyl-L-allylglycine for N-tert-butoxycarbonyl-L-leucine in step (b), the title compound was prepared as a white solid (0.105 g, 74%). MS (ESI): 522.1 (M+H)⁺.

### Example 135

### Preparation of N-[2-(1-naphthyl)thiazol-4-ylcarbonyl]-N′-[N-(2-quinolinoyl)-L-b-cyclopropylalanyl]hydrazide

Following the procedure of Example 59(a)-59(d), except substituting N-tert-butoxycarbonyl-L-b-cyclopropylalanine for N-tert-butoxycarbonyl-L-leucine in step (b) and 2-quinolinecarboxylic acid for 8-quinolinecarboxylic acid in step (d), the title compound was prepared as a white solid (0.151 g, 86%). MS (ESI): 536.3 (M+H)⁺.

### Example 136

### Preparation of N-[N-(3-isoquinolinoyl)-L-b-cyclopropylalanyl]-N′[-[2-(1-naphthyl)thiazol-4-ylcarbonyl]hydrazide

Following the procedure of Example 59(a)-59(d), except substituting N-tert-butoxycarbonyl-L-cyclopropylalanine for N-tert-butoxycarbonyl-L-leucine in step (b) and 3-isoquinolinecarboxylic acid for 8-quinolinecarboxylic acid in step (d), the title compound was prepared as a white solid (0.145 g, 82%). MS (ESI): 536.1 (M+H)⁺.

### Example 137

### Preparation of N-[N-(1-isoquinolinoyl)-L-b-cyclopropylalanyl]-N′-[2-(1-naphthyl)thiazol-4-ylcarbonyl]hydrazide

Following the procedure of Example 59(a)-59(d), except substituting N-tert-butoxycarbonyl-L-b-cyclopropylalanine for N-tert-butoxycarbonyl-L-leucine in step (b) and 1-isoquinolinecarboxylic acid for 8-quinolinecarboxylic acid in step (d), the title compound was prepared as a white solid (0.143 g, 81%). MS (ESI): 536.1 (M+H)⁺.

### Example 138

### Preparation of N-[2-(1-naphthyl)thiazol-4-ylcarbonyl]-N′-[N-(7-quinolinoyl)-L-b-cyclopropylalanyl]hydrazide

Following the procedure of Example 59(a)-59(d), except substituting N-tert-butoxycarbonyl-L-b-cyclopropylalanine for N-tert-butoxycarbonyl-L-leucine in step (b) and 7-quinolinecarboxylic acid for 8-quinolinecarboxylic acid in step (d), the title compound was prepared as a white solid (0.138 g, 78%). MS (ESI): 536.1 (M+H)⁺.

### Example 139

### Preparation of N-[2-(N-cyclopropyl-N-cyclopropylmethylamino)thiazol-4-ylcarbonyl]-N′-[N-(8-quinolinoyl)-L-b-cyclopropylalanyl]hydrazide

Following the procedure of Example 56(a)-56(b), except substituting N-(N-tert-butoxycarbonyl-L-b-cyclopropylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 8-quinolinecarboxylic acid for picolinic acid in step (b), the title compound was prepared as a white solid (120 mg, 73%). MS (ESI): 519.1 (M+H)⁺.

### Example 140

### Preparation of N-[2-(N-cyclopropyl-N-cyclopropylmethylamino)thiazol-4-ylcarbonyl]-N′-[N-(4-methylimidazol-5-ylcarbonyl)-L-b-cyclopropylalanyl]hydrazide

Following the procedure of Example 56(a)-56(b), except substituting N-(N-tert-butoxycarbonyl-L-b-cyclopropylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 4-methylimidazole-5-carboxylic acid for picolinic acid in step (b), the title compound was prepared as a white solid (120 mg, 81%). MS (ESI): 472.1 (M+H)⁺.

### Example 141

### Preparation of N-[2-(N-cyclopropyl-N-cyclopropylmethylamino)thiazol-4-ylcarbonyl]-N′-[N-(3-isoquinolinoyl)-L-b-cyclopropylalanyl]hydrazide

Following the procedure of Example 56(a)-56(b), except substituting N-(N-tert-butoxycarbonyl-L-b-cyclopropylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 3-isoquinolinecarboxylic acid for picolinic acid in step (b), the title compound was prepared obtained as a white solid (140 mg, 82%). MS (ESI): 519.2 (M+H)⁺.

### Example 142

### Preparation of N-[2-(N-cyclopropyl-N-cyclopropylmethylamino)thiazol-4-ylcarbonyl]-N′-[N-(6-methylnicotinoyl)-L-b-cyclopropylalanyl]hydrazide

Following the procedure of Example 56(a)-56(b), except substituting N-(N-tert-butoxycarbonyl-L-b-cyclopropylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 6-methylnicotinic acid for picolinic acid in step (b), the title compound was prepared as a white solid (105 mg, 62%). MS (ESI): 483.2 (M+H)⁺.

### Example 143

### Preparation of N-[N-(4-methylimidazol-5-ylcarbonyl)-L-norleucinyl]-N′-[2-(1-naphthyl)thiazol-4-ylcarbonyl]hydrazide

Following the procedure of Example 59(a)-59(d), except substituting N-tert-butoxycarbonyl-L-norleucine for N-tert-butoxycarbonyl-L-leucine in step (b) and 4-methylimidazole-5-carboxylic acid for 8-quinolinecarboxylic acid in step (d), the title compound was prepared as a white solid (0.112 g,70%). MS (ESI): 491.1 (M+H)⁺.

### Example 144

### Preparation of N-[2-(1-naphthyl)thiazol-4-ylcarbonyl]-N′-(N-picolinoyl-L-norleucinyl)hydrazide

Following the procedure of Example 59(a)-59(d), except substituting N-tert-butoxycarbonyl-L-norleucine for N-tert-butoxycarbonyl-L-leucine in step (b) and picolinic acid for 8-quinolinecarboxylic acid in step (d), the title compound was prepared as a white solid (0.114 g, 72%). MS (ESI): 488.2 (M+H)⁺.

### Example 145

### Preparation of N-[2-(1-naphthyl)thiazol-4-ylcarbonyl]-N′-[N-(8-quinolinoyl)-L-norleucinyl]hydrazide

Following the procedure of Example 59(a)-59(d), except substituting N-tert-butoxycarbonyl-L-norleucine for N-tert-butoxycarbonyl-L-leucine in step (b), the title compound was prepared as a white solid (0.082 g, 47%). MS (ESI): 538.1 (M+H)⁺.

### Example 146

### Preparation of N-[2-(N-cyclopropyl-N-cyclopropylmethylamino)thiazol-4-ylcarbonyl]-N′-[N-(2-quinolinoyl)-L-b-cyclopropylalanyl]hydrazide

Following the procedure of Example 56(a)-56(b), except substituting N-(N-tert-butoxycarbonyl-L-b-cyclopropylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 2-quinolinecarboxylic acid for picolinic acid in step (b), the title compound was prepared as a white solid (150 mg, 81%). MS (ESI): 519.2 (M+H)⁺.

### Example 147

### Preparation of N-[2-(N-cyclopropyl-N-cyclopropylmethylamino)thiazol-4-ylcarbonyl]-N′-[N-(1-isoquinolinoyl)-L-b-cyclopropylalanyl]hydrazide

Following the procedure of Example 56(a)-56(b), except substituting N-(N-tert-butoxycarbonyl-L-b-cyclopropylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 1-isoquinolinecarboxylic acid for picolinic acid in step (b), the title compound was prepared as a white solid (130 mg, 87%). MS (ESI): 519.2 (M+H)⁺.

### Example 148

### Preparation of N-[2-[N-cyclopropyl-N-(2-methoxypropyl)amino]thiazol-4-ylcarbonyl]-N′-[N-(6-methyl-3-pyridinylmethoxycarbonyl)-L-leucinyl]hydrazide

Following the procedure of Example 1(a)-1(h), except substituting N-isobutyl cyclopropylamine for cis-2,6-dimethylmorpholine in step (a) and 6-methyl-3-pyridylcarbinol for 4-pyridylcarbinol in step (f), the title compound was prepared as a white solid (220 mg, 88%). MS (ESI): 517.2 (M+H)⁺.

### Example 149

### Preparation of N-(N-tert-butoxycarbonyl-L-leucinyl)-N′-[2-[N-cyclopropyl-N-(2-methylpropyl)amino]thiazol-4-ylcarbonyl]hydrazide

Following the procedure of Example 1(a)-1(d) and 1(h), except substituting N-cyclopropyl isobutylamine for cis-2,6-dimethylmorpholine in step (a) and N-tert-butoxycarbonyl-L-leucine for N-(4-pyridinylmethoxycarbonyl)-L-leucine in step (h), the title compound was prepared as a white solid (1.01 g, 89%). MS (ESI): 466.3 (M+H)⁺.

### Example 150

### Preparation of N-[2-(1-naphthyl)thiazol-4-ylcarbonyl]-N′-[N-(7-quinolinoyl)-L-b-tert-butylalanyl]hydrazide

Following the procedure of Example 59(a)-59(d), except substituting N-tert-butoxycarbonyl-L-b-tert-butylalanine for N-tert-butoxycarbonyl-L-leucine in step (b) and 7-quinolinecarboxylic acid for 8-quinolinecarboxylic acid in step (d), the title compound was prepared as a white solid (0.139 g, 80%). MS (ESI): 552.2 (M+H)⁺.

### Example 151

### Preparation of N-[2-(1-naphthyl)thiazol-4-ylcarbonyl]-N′-[N-(2-quinolinoyl)-L-b-tert-butylalanyl]hydrazide

Following the procedure of Example 59(a)-59(d), except substituting N-tert-butoxycarbonyl-L-b-tert-butylalanine for N-tert-butoxycarbonyl-L-leucine in step (b) and 2-quinolinecarboxylic acid for 8-quinolinecarboxylic acid in step (d), the title compound was prepared as a white solid (0.158 g, 91%). MS (ESI): 552.2 (M+H)⁺.

### Example 152

### Preparation of N-[N-(1-isoquinolinoyl)-L-b-tert-butylalanyl]-N′-[2-(1-naphthyl)thiazol-4-ylcarbonyl]hydrazide

Following the procedure of Example 59(a)-59(d), except substituting N-tert-butoxycarbonyl-L-b-tert-butylalanine for N-tert-butoxycarbonyl-L-leucine in step (b) and 1-isoquinolinecarboxylic acid for 8-quinolinecarboxylic acid in step (d), the title compound was prepared as a white solid (0.143 g, 82%). MS (ESI): 552.2 (M+H)⁺.

### Example 153

### Preparation of N-[N-(3-isoquinolinoyl)-L-b-tert-butylalanyl]-N′-[2-(1-naphthyl)thiazol-4-ylcarbonyl]hydrazide

Following the procedure of Example 59(a)-59(d), except substituting N-tert-butoxycarbonyl-L-b-tert-butylalanine for N-tert-butoxycarbonyl-L-leucine in step (b) and 3-isoquinolinecarboxylic acid for 8-quinolinecarboxylic acid in step (d), the title compound was prepared as a white solid (0.130 g, 75%). MS (ESI): 552.2 (M+H)⁺.

### Example 154

### Preparation of N-[N-(6-methylnicotinoyl)-L-norleucinyl]-N′-[2-(1-naphthyl)thiazol-4-ylcarbonyl]hydrazide

Following the procedure of Example 59(a)-59(d), except substituting N-tert-butoxycarbonyl-L-norleucine for N-tert-butoxycarbonyl-L-leucine in step (b) and 6-methylnicotinic acid for 8-quinolinecarboxylic acid in step (d), the title compound was prepared as a white solid (0.109 g, 67%). MS (ESI): 502.2 (M+H)⁺.

### Example 155

### Preparation of N-[2-(1-naphthyl)thiazol-4-ylcarbonyl]-N′-[N-(7-quinolinoyl)-L-norleucinyl]hydrazide

Following the procedure of Example 59(a)-59(d), except substituting N-tert-butoxycarbonyl-L-norleucine for N-tert-butoxycarbonyl-L-leucine in step (b) and 7-quinolinecarboxylic acid for 8-quinolinecarboxylic acid in step (d), the title compound was prepared as a white solid (0.104 g, 59%). MS (ESI): 538.1 (M+H)⁺.

### Example 156

### Preparation of N-[2-(1-naphthyl)thiazol-4-ylcarbonyl]-N′-[N-(2-quinolinoyl-L-norleucinyl]hydrazide

Following the procedure of Example 59(a)-59(d), except substituting N-tert-butoxycarbonyl-L-norleucine for N-tert-butoxycarbonyl-L-leucine in step (b) and 2-quinolinecarboxylic acid for 8-quinolinecarboxylic acid in step (d), the title compound was prepared as a white solid (0.153 g, 87%). MS (ESI): 538.1 (M+H)⁺.

### Example 157

### Preparation of N-[N-(1-isoquinolinoyl)-L-norleucinyl]-N′-[2-(1-naphthyl)thiazol-4-ylcarbonyl]hydrazide

Following the procedure of Example 59(a)-59(d), except substituting N-tert-butoxycarbonyl-L-norleucine for N-tert-butoxycarbonyl-L-leucine in step (b) and 1-isoquinolinecarboxylic acid for 8-quinolinecarboxylic acid in step (d), the title compound was prepared as a white solid (0.151 g, 86%). MS (ESI): 538.1 (M+H)⁺.

### Example 158

### Preparation of N-[N-(3-isoquinolinoyl)-L-norleucinyl]-N′-[2-(1-naphthyl)thiazol-4-ylcarbonyl]hydrazide

Following the procedure of Example 59(a)-59(d), except substituting N-tert-butoxycarbonyl-L-norleucine for N-tert-butoxycarbonyl-L-leucine in step (b) and 3-isoquinolinecarboxylic acid for 8-quinolinecarboxylic acid in step (d), the title compound was prepared as a white solid (0.126 g, 72%). MS (ESI): 538.1 (M+H)⁺.

### Example 159

### Preparation of N-[2-(N-cyclopropyl-N-cyclopropylmethylamino)thiazol-4-ylcarbonyl]-N′-[N-(5-hydroxymethylimidazol-4-ylcarbonyl)-L-b-cyclopropylalanyl]hydrazide

Following the procedure of Example 56(a)-56(b), except substituting N-(N-tert-butoxycarbonyl-L-b-cyclopropylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 5-hydroxymehylimidazole-4-carboxylic acid for picolinic acid in step (b), the title compound was prepared as a white solid (50 mg, 44%). MS (ESI): 488.2 (M+H)⁺.

### Example 160

### Preparation of N-[2-[N-cyclopropyl-N-(2-methylpropyl)amino]thiazol-4-ylcarbonyl]-N′-[N-(8-Quinolinoyl)-L-b-cyclopropylalanyl]hydrazide

### a) N-(N-tert-butoxycarbonyl-L-b-cyclopropylalanyl)-N′-[2-[N-cyclopropyl-N-(2-methylpropyl)amino]thiazol-4-ylcarbonyl]hydrazide

Following the procedure of Example 1(a)-1(d) and 1(h), except substituting N-cyclopropyl isobutylamine for cis-2,6-dimethylmorpholine in step (a) and N-tert-butoxycarbonyl-L-b-cyclopropylalanine for N-(4-pyridinylmethoxycarbonyl)-L-leucine in step (h), the title compound was prepared as a white solid (1.01 g, 89%). MS (ESI): 466.3 (M+H)⁺.

### b) N-[2-[N-cyclopropyl-N-(2-methylpropyl)amino]thiazol-4-ylcarbonyl]-N′-[N-(8-quinolinoyl)-L-b-cyclopropylalanyl]hydrazide

Following the procedure of Example 56(a)-56(b), except substituting N-(N-tert-butoxycarbonyl-L-b-cyclopropylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 8-quinolinecarboxylic acid for picolinic acid in step (b), the title compound was prepared as a white solid (135 mg, 100%). MS (ESI): 521.2 (M+H)⁺.

### Example 161

### Preparation of N-[2-(N-cyclopropyl-N-cyclopropylmethylamino)thiazol-4-ylcarbonyl]-N′-(N-(6-methylnicotinoyl)-L-b-tert-butylalanyl]hydrazide

Following the procedure of Example 56(a)-56(b), except substituting N-[2-(N-cyclopropyl-N-cyclopropylmethylamino)thiazol-4-ylcarbonyl]-N′-(N-tert-butoxycarbonyl-L-b-tert-butylalanyl)hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 6-methylnicotinic acid for picolinic acid in step (b), the title compound was prepared as a white solid (85 mg, 79%). MS (ESI): 499.2 (M+H)⁺.

### Example 162

### Preparation of N-[2-[N-cyclopropyl-N-(2-methylpropyl)amino]thiazol-4-ylcarbonyl]-N′-[N-(4-methylimidazol-5-ylcarbonyl)-L-b-cyclopropylalanyl]hydrazide

Following the procedure of Example 56(a)-56(b), except substituting N-(N-tert-butoxycarbonyl-L-b-cyclopropylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 4-methylimidazole-5-carboxylic acid for picolinic acid in step (b), the title compound was prepared as a white solid (100 mg, 73%). MS (ESI): 474.2 (M+H)⁺.

### Example 163

### Preparation of N-[2-[N-cyclopropyl-N-(2-methylpropyl)amino]thiazol-4-ylcarbonyl]-N′-[N-(2-quinolinoyl)-L-b-cyclopropylalanyl]hydrazide

Following the procedure of Example 56(a)-56(b), except substituting N-(N-tert-butoxycarbonyl-L-b-cyclopropylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 2-quinolinecarboxylic acid for picolinic acid in step (b), the title compound was prepared as a white solid (75 mg, 59%). MS (ESI): 521.2 (M+H)⁺.

### Example 164

### Preparation of N-[2-[N-cyclopropyl-N-(2-methylpropyl)amino]thiazol-4-ylcarbonyl]-N′-[N-(6-methylnicotinoyl)-L-b-cyclopropylalanyl]hydrazide

Following the procedure of Example 56(a)-56(b), except substituting N-(N-tert-butoxycarbonyl-L-b-cyclopropylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 6-methylnicotinic acid for picolinic acid in step (b), the title compound was prepared as a white solid (112 mg, 65%). MS (ESI): 485.3 (M+H)⁺.

### Example 165

### Preparation of N-[2-(1-naphthyl)thiazol-4-ylcarbonyl]-N′-[N-(8-quinolinoyl)glycinyl]hydrazide

### a) N-(8-quinolinoyl)glycine

Following the procedure of Example 102(f)-102(g), except substituting glycine methyl ester hydrochloride for L-leucine methyl ester in step (f), the title compound was prepared as a pale yellow solid (0.207 g, 95%). MS (ESI): 231.1 (M+H)⁺.

### b) N-[2-(1-naphthyl)thiazol-4-ylcarbonyl]-N′-[N-(8-quinolinoyl)glycinyl]hydrazide

Following the procedure of Example 1(h), except substituting N-[2-(1-naphthyl)thiazol-4-ylcarbonyl]hydrazide for N-[2-(cis-2,6-dimethyl-4-morpholino)thiazol-4-ylcarbonyl]hydrazide and N-(8-quinolinoyl)glycine for N-(4-pyridylmethoxycarbonyl)-L-leucine, the title compound was prepared as a tan solid (0.028 g, 12%). MS (ESI): 482.1 (M+H)⁺.

### Example 166

### Preparation of N-[2-(1-naphthyl)thiazol-4-ylcarbonyl]-N′-[N-(8-quinolinoyl)-L-norvalinyl]hydrazide

Following the procedure of Example 59(a)-59(d), except substituting N-tert-butoxycarbonyl-L-norvaline for N-tert-butoxycarbonyl-L-leucine in step (b), the title compound was prepared as a white solid (0.131 g, 74%). MS (ESI): 524.1 (M+H)⁺.

### Example 167

### Preparation of N-[2-(1-naphthyl)thiazol-4-ylcarbonyl]-N′-[N-(2-quinolinoyl)-L-norvalinyl]hydrazide

Following the procedure of Example 59(a)-59(d), except substituting N-tert-butoxycarbonyl-L-norvaline for N-tert-butoxycarbonyl-L-leucine in step (b) and 2-quinolinecarboxylic acid for 8-quinolinecarboxylic acid in step (d), the title compound was prepared as a white solid (0.135 g, 75%). MS (ESI): 524.1 (M+H)⁺.

### Example 168

### Preparation of N-[2-(1-naphthyl)thiazol-4-ylcarbonyl]-N′-(N-picolinoyl-L-norvalinyl]hydrazide

Following the procedure of Example 59(a)-59(d), except substituting N-tert-butoxycarbonyl-L-norvaline for N-tert-butoxycarbonyl-L-leucine in step (b) and picolinic acid for 8-quinolinecarboxylic acid in step (d), the title compound was prepared as a white solid (0.126 g, 79%). MS (ESI): 474.2 (M+H)⁺.

### Example 169

### Preparation of N-[2-(1-naphthyl)thiazol-4-ylcarbonyl]-N′-[N-(6-methylnicotinoyl)-L-norvalinyl]hydrazide

Following the procedure of Example 59(a)-59(d), except substituting N-tert-butoxycarbonyl-L-norvaline for N-tert-butoxycarbonyl-L-leucine in step (b) and 6-methylnicotinic acid for 8-quinolinecarboxylic acid in step (d), the title compound was prepared as a white solid (0.141 g, 85%). MS (ESI): 488.2 (M+H)⁺.

### Example 170

### Preparation of N-[2-(1-naphthyl)thiazol-4-ylcarbonyl]-N′-[N-(4-methylimidazol-5-ylcarbonyl)-L-norvalinyl]hydrazide

Following the procedure of Example 59(a)-59(d), except substituting N-tert-butoxycarbonyl-L-norvaline for N-tert-butoxycarbonyl-L-leucine in step (b) and 4-methylimidazole-5-carboxylic acid for 8-quinolinecarboxylic acid in step (d), the title compound was prepared as a white solid (0.098 g, 51%). MS (ESI): 477.1 (M+H)⁺.

### Example 171

### Preparation of N-[2-(1-naphthyl)thiazol-4-ylcarbonyl]-N′-[N-(1-isoquinolinoyl)-L-norvalinyl]hydrazide

Following the procedure of Example 59(a)-59(d), except substituting N-tert-butoxycarbonyl-L-norvaline for N-tert-butoxycarbonyl-L-leucine in step (b) and 1-isoquinolinecarboxylic acid for 8-quinolinecarboxylic acid in step (d), the title compound was prepared as a white solid (0.146 g, 82%). MS (ESI): 524.2 (M+H)⁺.

### Example 172

### Preparation of N-[2-(1-naphthyl)thiazol-4-ylcarbonyl]-N′-[N-(3-isoquinolinoyl)-L-norvalinyl]hydrazide

Following the procedure of Example 59(a)-59(d), except substituting N-tert-butoxycarbonyl-L-norvaline for N-tert-butoxycarbonyl-L-leucine in step (b) and 3-isoquinolinecarboxylic acid for 8-quinolinecarboxylic acid in step (d), the title compound was prepared as a white solid (0.138 g, 78%). MS (ESI): 524.2 (M+H)⁺.

### Example 173

### Preparation of (1S, 1′S)-N,N′-bis-[4-[1-(N-benzyloxycarbonylamino)-3-methylbutyllthiazol-2-ylcarbonyl]hydrazide

### a) N-benzyloxycarbonyl-L-leucinamide

To a stirring solution of N-benzyloxycarbonyl-L-leucine (4.6 g, 17.3 mmol) in THF, cooled to −40° C., was added N-methylmorpholine (3.68 g, 36.4 mmol; 4.0 mL) and isobutyl chloroformate (2.37 g, 17.3 mmol; 2.25 mL). After stirring for 15 min, ammonia was bubbled through the solution for 5 min. The solution was warmed to room temperature, evaporated, and the residue was dissolved in ethyl acetate, washed with 0.1 N Hcl, and saturated brine, then dried (MgSO₄), filtered and evaporated to dryness to give the title compound as a white solid (4.58 g, 100%).

### b) N-benzyloxycarbonyl-L-leucinethioamide

A solution of the compound of Example 1(a) (4.58 g, 17.3 mmol) and Lawesson's reagent (4.21 g, 10.4 mmol) in THF was allowed to stir at room temperature for 16 h. The solution was concentrated and the residue was purified by flash chromatography on 230-400 mesh silica gel, eluting with 1:3 EtOAc/hexanes, to provide the title compound as a pale yellow solid (3.74 g, 77%).

### c) (1S)-1-benzyloxycarbonylamino-1-(4-carboethoxythiazol-2-yl)-3-methylbutane

The compound of Example 1(b) (2.20 g, 7.83 mmol) was dissolved in acetone (35 mL), cooled to −10° C., and ethyl bromopyruvate (1.68 g, 8.62 mmol, 1.08 mL) was added.

After stirring for 1 h, the solution was poured into methylene chloride/water, then into saturated aqueous NaHCO₃. The aqueous layer was extracted with methylene chloride and the combined organic layers were washed with saturated brine, dried (MgSO₄), filtered and concentrated. The residue was dissolved in methylene chloride, cooled to −20° C., pyridine (1.36 g, 17.2 mmol, 1.39 mL) and trifluroracetic anhydride (1.81 g, 8.62 mmol, 1.22 mL ) were added. After stirring for 1 h, the solution was washed with saturated squeous NaNCO₃ and saturated brine, then dired (MgSO₄), filtered, and concentrated. The residue was purified by flash chromatography on 90 g of 230-400 mesh silica gel, eluting with 1:3 ethyl acetate/hexanes, to provide the title compound as a pale yellow oil (2.36 g, 80%). ¹H NMR (400 MHz, CDCl₃) d 8.08 (s, 1H), 7.38 (m, 5H), 5.42 (s, 3H), 5.23-5.07 (m, 3H), 4.42 (q, 2H), 2.01-1.62 (m, 3H), 1.41 (t, 3H), 0.99 (d, 6H).

### d) (1S)-benzyloxycarbonylamino-1-(4-hydrazinocarbonylthiazol-2-yl)-3-methylbutane

Following the procedure of Example 1(d), except substituting (1S)-1-benzyloxycarbonylamino-1-(4-carboethoxythiazol-2-yl)-3-methylbutane for ethyl 2-(cis-2,6-dimethyl-4-morpholino)thiazole-4-carboxylate, the title compound was prepared as a pale yellow foam (2.01 g, 97%). ¹H NMR (400 MHz, CDCl₃) d 8.35 (bs, 1H), 8.03 (s, 1H), 7.37 (m, 5H), 5.29 (d, 1H), 5.14-5.09 (m, 3H), 4.07 (bs, 2H), 1.92-1.82 (m, 1H), 1.79-1.66 (m, 2H), 1.00 (d, 6H).

### e) (1S)-1-benzyloxycarbonylamino-1-(4-carboxythiazol-2-yl)-3-methylbutane

Following the procedure of Example 1(g), except substituting (1S)-1-benzyloxycarbonylamino-1-(4-carboethoxythiazol-2-yl)-3-methylbutane for N-(4-pyridinylmethoxycarbonyl)-L-leucine methyl ester, the title compound was prepared as a white solid. MS (ESI): 349.2 (M+H)⁺.

### f) (1S, 1′S)-N,N′-bis-[4-[1-(N-benzyloxycarbonylamino)-3-methylbutyl]thiazol-2-ylcarbonyl]hydrazide

Following the procedure of Example 1(h), except substituting (1S)-1-benzyloxycarbonylamino-1-(4-hydrazinocarbonylthiazol-2-yl)-3-methylbutane for N-[2-(cis-2,6-dimethyl-4-morpholino)thiazol-4-ylcarbonyl]hydrazide and (1S)-1-benzyloxycarbonylamino-1-(4-carboxythiazol-2-yl)-3-methylbutane for N-(4-pyridylmethoxycarbonyl)-L-leucine, the title compound was prepared as a white solid (0.028 g, 59%). MS (ESI): 693.1 (M+H)⁺.

### Example 174

### Preparation of N-[2-[N-cyclopropyl-N-(2-methylpropyl)amino]thiazol-4-ylcarbonyl]-N′-[N-(6-methylnicotinoyl)-L-b-tert-butylalanyl]hydrazide

### a) N-(N-tert-butoxycarbonyl-L-b-tert-butylalanyl)-N′-[2-[N-cyclopropyl-N-(2-methylpropyl)amino]thiazol-4-ylcarbonyl]hydrazide

Following the procedure of Example 1(a)-1(d) and 1(h), except substituting N-cyclopropyl isobutylamine for cis-2,6-dimethylmorpholine in step (a) and N-tert-butoxycarbonyl-L-b-tert-butylalanine for N-(4-pyridinylmethoxycarbonyl)-L-leucine in step (h), the title compound was prepared as a white solid (0.44 g, 100%). MS (ESI): 482.3 (M+H)⁺.

### b) N-[2-[N-cyclopropyl-N-(2-methylpropyl)amino]thiazol-4-ylcarbonyl]-N′-[N-(6-methylnicotinoyl)-L-b-tert-butylalanyl]hydrazide

Following the procedure of Example 56(a)-56(b), except substituting N-(N-tert-butoxycarbonyl-L-btert-butylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 6-methylnicotinic acid for picolinic acid in step (b), the title compound was prepared as a white solid (70 mg, 66%). MS (ESI): 501.2 (M+H)⁺.

### Example 175

### Preparation of N-[2-[N-cyclopropyl-N-(2-methylpropyl)amino]thiazol-4-ylcarbonyl]-N′-[N-(4-methylimidazol-5-ylcarbonyl)-L-b-tert-butylalanyl]hydrazide

Following the procedure of Example 56(a)-56(b), except substituting N-(N-tert-butoxycarbonyl-L-b-tert-butylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 4-methylimidazole-5-carboxylic acid for picolinic acid in step (b), the title compound was prepared as a white solid (70 mg, 39%). MS (ESI): 490.2 (M+H)⁺.

### Example 176

### Preparation of N-[2-(N-cyclopropyl-N-cyclopropylmethylamino)thiazol-4-ylcarbonyl]-N′-[N-(1-isoquinolinoyl)-L-b-tert-butylalanyl]hydrazide

Following the procedure of Example 56(a)-56(b), except substituting N-(N-tert-butoxycarbonyl-L-b-tert-butylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 1-isoquinolinecarboxylic acid for picolinic acid in step (b), the title compound was prepared as a white solid (123 mg, 88%). MS (ESI): 535.3 (M+H)⁺.

### Example 177

### Preparation of N-[N-(5-butylpicolinoyl)-L-b-tert-butylalanyl]-N′-[2-(N-cyclopropyl-N-cyclopropylmethylamino)thiazol-4-ylcarbonyl]hydrazide

Following the procedure of Example 56(a)-56(b), except substituting N-(N-tert-butoxycarbonyl-L-b-tert-butylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 5-butylpicolinic acid for picolinic acid in step (b), the title compound was prepared as a white solid (90 mg, 85%). MS (ESI): 541.3 (M+H)⁺.

### Example 178

### Preparation of N-[2-(Ncyclopropyl-N-cyclopropylmethylamino)thiazol-4-ylcarbonyl]-N′-[N-(6-methylpicolinoyl)-L-b-tert-butylalanyl]hydrazide

Following the procedure of Example 56(a)-56(b), except substituting N-(N-tert-butoxycarbonyl-L-b-tert-butylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 6-methylpicolinic acid for picolinic acid in step (b), the title compound was prepared as a white solid (170 mg, 86%). MS (ESI): 499.2 (M+H)⁺.

### Example 179

### Preparation of N-[2-(N-cyclopropyl-N-cyclopropylmethylamino)thiazol-4-ylcarbonyl]-N′-[N-(4-fluorobenzoyl)-L-leucinyl)hydrazide

Following the procedure of Example 56(a)-56(b), except substituting 4-fluorobenzoic acid for picolinic acid in step (b), the title compound was prepared as a white solid (88 mg, 97%). MS (ESI): 488.2 (M+H)⁺.

### Example 180

### Preparation of N-[N-(4-fluorobenzoyl)-L-leucinyl]-N′-[2-(1-naphthyl)thiazol-4-ylcarbonyl]hydrazide

Following the procedure of Example 59(a)-59(d), except substituting 4-fluorobenzoic acid for 8-quinolinecarboxylic acid in step (d), the title compound was prepared as a white solid (0.113 g, 69%). MS (ESI): 505.1 (M+H)⁺.

### Example 181

### Preparation of N-[2-(1-naphthyl)thiazol-4-ylcarbonyl]-N′-[N-(2-pyridinylmethoxycarbonyl)-L-b-tert-butylalanyl]hydrazide

### a)-L-b-tert-butylalanine Methyl Ester Hydrochloride

To a suspension of L-b-tert-butylalanine (2.0 g, 13.8 rnrnol) in 2,2-dimethoxypropane (75 mL) was added concentrated hydrochloric acid (12 mL). After standing at room temperature for 16 h, the solution was concentrated, redissolved in ethyl acetate and washed with 7.5% Na₂CO₃ (2×). The organic layer was dried (MgSO₄), filtered and concentrated to yield the free base (1.3 g, 8.2 mmol). This was dissolved in ether and HCl (8.2 mL, 1.0 M in ether) added. The white precipitate was collected by filtration yield the title compound as a white solid (1.32 g, 49%). MS (ESI): 159.7 (M+H)⁺.

### b) N-(2-pyridinylmethoxycarbonyl)-L-b-tert-butylalanine

Following the procedure of Example 1(e)-5(g), except substituting L-b-tert-butylalanine methyl ester hydrochloride for L-leucine methylo ester hydrochloride in step (e) and 2-pyridylcarbinol for 4-pyridylcarbinol in step (f), the title compound was prepared as a white solid (0.55 g, 100%). MS (ESI): 281.3 (M+H)⁺.

### c) N-[2-(1-naphthyl)thiazol-4-ylcarbonyl]-N′-[N-(2-pyridinylmethoxycarbonyl)-L-b-tert-butylalanyl]hydrazide

Following the procedure of Example 1(h), except substituting N-[2-(1-naphthyl)thiazol-4-ylcarbonyl]hydrazide for N-[2-(cis-2,6-dimethyl-4-morpholino)thiazol-4-ylcarbonyl]hydrazide and N-(2-pyridinylmethoxycarbonyl)-L-b-tert-butylalanine for N-(4-pyridylmethoxycarbonyl)-L-leucine, the title compound was prepared as a white solid (0.155 g, 47%). MS (ESI): 532.2 (M+H)⁺.

### Example 182

### Preparation of N-[N-(2-methyl-3-pyridinylmethoxycarbonyl)-L-b-tert-butylalanyl]-N′-[2-(1-naphthyl)thiazol-4-ylcarbonyl]hydrazide

Following the procedure of Example 181(a)-181(c), except substituting 2-methyl-3-pyridylcarbinol for 2-pyridylcarbinol in step (b), the title compound was prepared as a white solid (0.169 g, 67%). MS (ESI): 546.2 (M+H)⁺.

### Example 183

### Preparation of N-[2-(1-naphthyl)thiazol-1-ylcarbonyl]-N′-[N-(2-pyridinylmethoxycarbonyl)-L-b-cyclopropylalanyl]hydrazide

### a)-L-b-cyclopropylalanine Methyl Ester Hydrochloride

Following the procedure of Example 181(a), except substituting N-tert-butoxycarbonyl-L-b-cyclopropylalanine methyl ester for L-b-tert-butylalanine, the title compound was prepared as a white solid (2.2 g, 30%). MS (ESI): 144.0 (M+H)⁺.

### b) N-[2-(1-naphthyl)thiazol-4-ylcarbonyl]-N′-[N-(2-pyridinylmethoxycarbonyl)-L-b-cyclopropylalanyl]hydrazide

Following the procedure of Example 181(b)-181(c), except substituting L-b-cyclopropylalanine methyl ester hydrochloride for L-b-tert-butylalanine methyl ester hydrochloride in step (b), the title compound was prepared as a white solid (0.147 g, 61%). MS (ESI): 516.1 (M+H)⁺.

### Example 184

### Preparation of N-[N-(2-methyl-3-pyridinylmethoxycarbonyl)-L-b-cyclopropylalanyl]-N′-[2-(1-naphthyl)thiazol-4-ylcarbonyl]hydrazide

Following the procedure of Example 181(a)-181(c), except substituting N-tert-butoxycarbonyl-L-b-cyclopropylalanine methyl ester for L-b-tert-butylalanine in step (a) and 2-methyl-3-pyridylcarbinol for 2-pyridylcarbinol in step (b), the title compound was prepared as a white solid (0.159 g, 65%). MS (ESI): 530.2 (M+H)⁺.

### Example 185

### Preparation of N-[N-(6-methyl-3-pyridinylmethoxycarbonyl)-L-b-cyclopropylalanyl]-N′-[2-(1-naphthyl)thiazol-4-ylcarbonyl]hydrazide

Following the procedure of Example 181(a)-181(c), except substituting N-tert-butoxycarbonyl-L-b-cyclopropylalanine methyl ester for L-b-tert-butylalanine in step (a) and 6-methyl-3-pyridylcarbinol for 2-pyridylcarbinol in step (b), the title compound was prepared as a white solid (0.169 g, 69%). MS (ESI): 530.2 (M+H)⁺.

### Example 186

### Preparation of N-[N-(6-methyl-3-pyridinylmethoxycarbonyl)-L-b-tert-butylalanyl]-N′-[2-(1-naphthyl)thiazol-4-ylcarbonyl]hydrazide

Following the procedure of Example 181(a)-181(c), except substituting 6-methyl-3-pyridylcarbinol for 2-pyridylcarbinol in step (b), the title compound was prepared as a white solid (0.194 g, 77%). MS (ESI): 546.2 (M+H)⁺.

### Example 187

### Preparation of N,N′-bis-[2-(1-naphthyl)thiazol-4-ylcarbonyl]hydrazide

### a) ethyl 2-(1-naphthyl)thiazole-4-carbohydrazide

Following the procedure of Example 3(a)-3(c) and 3(e), except substituting 1-naphthylboronic acid for 4-methyl-1-naphthylboronic acid in step (e), the title compound was prepared as a pale yellow solid. MS (ESI): 270.1 (M+H)⁺.

### a) ethyl 2-(1-naphthyl)thiazole-4-carbohydrazide

Following the procedure of Example 1(g), except substituting ethyl 2-(1-naphthyl)thiazole-4-carbohydrazide for N-(4-pyridinylmethoxycarbonyl)-L-leucine methyl ester, the title compound was prepared as a white solid. MS (ESI): 256.0 (M+H)⁺.

### Example 188

### Preparation of N-[2-(N-cyclopropyl-N-cyclopropylmethylamino)thiazol-4-ylcarbonyl]-N′-[N-[2-(1,8-naphthyridinoyl)]-L-b-cyclopropylalanyl]hydrazide

Following the procedure of Example 56(a)-56(b), except substituting N-(N-tert-butoxycarbonyl-L-b-cyclopropylalanyl)-N′-[2-(N-cyclopropyl-N-cyclopropylmethylamino)thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 1,8-naphthyridine-2-carboxylic acid for picolinic acid in step (b), the title compound was prepared as a white solid (100 mg, 59%). MS (ESI): 520.2 (M+H)⁺.

### Example 189

### Preparation of N-[2-[N-cyclopropyl-N-(2-methylpropyl)amino]thiazol-4-ylcarbonyl]-N′-[N-(3,4-difluorobenzoyl)-L-b-cyclopropylalanyl]hydrazide

Following the procedure of Example 56(a)-56(b), except substituting N-(N-tert-butoxycarbonyl-L-b-cyclopropylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 3,4-difluorobenzoic acid for picolinic acid in step (b), the title compound was prepared as a white solid (208 mg, 100%). MS (ESI): 506.1 (M+H)⁺.

### Example 190

### Preparation of N-[2-[N-cyclopropyl-N-(2-methylpropyl)amino]thiazol-4-ylcarbonyl]-N′-[N-(4-flluorobenzoyl)-L-leucinyl]hydrazide

Following the procedure of Example 56(a)-56(b), except substituting N-(N-tert-butoxycarbonyl-L-leucinyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 4-fluorobenzoic acid for picolinic acid in step (b), the title compound was prepared as a white solid (130 mg, 70%). MS (ESI): 490.2 (M+H)⁺.

### Example 191

### Preparation of N-[N-(5-butylpicolinoyl)-L-leucinyl]-N′-[2-(N-cyclopropyl-N-cyclopropylmethylamino)thiazol-4-ylcarbonyl]hydrazide

Following the procedure of Example 56(a)-56(b), except substituting N-(N-tert-butoxycarbonyl-L-leucinyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 5-butylpicolinic acid for picolinic acid in step (b), the title compound was prepared as a white solid (100 mg, 63%). MS (ESI): 529.3 (M+H)⁺.

### Example 192

### Preparation of N-[2-[N-cyclopropyl-N-(2-methylpropyl)amino]thiazol-4-ylcarbonyl]-N′-[N-(3,4-dimethoxybenzoyl)-L-leucinyl]hydrazide

Following the procedure of Example 56(a)-56(b), except substituting N-(N-tert-butoxycarbonyl-L-leucinyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 3,4-dimethoxybenzoic acid for picolinic acid in step (b), the title compound was prepared as a white solid (130 mg, 84%). MS (EIS): 532.2 (M+H)⁺.

### Example 193

### Preparation of N-[2-[N-cyclopropyl-N-(2-methylpropyl)amino]thiazol-4-ylcarbonyl]-N′-[N-(3,4-difluorobenzoyl)-L-b-tert-butylalanyl]hydrazide

Following the procedure of Example 56(a)-56(b), except substituting N-(N-tert-butoxycarbonyl-L-b-tert-butylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 3,4-difluorobenzoic acid for picolinic acid in step (b), the title compound was prepared as a white solid (120 mg, 78%). MS (ESI): 522.2 (M+H)⁺.

### Example 194

### Preparation of N-[2-[N-cyclopropyl-N-(2-methylpropyl)amino]thiazol-4-ylcarbonyl]-N′-[N-(3,4-dimethoxybenzoyl)-L-b-tert-butylalanyl]hydrazide

Following the procedure of Example 56(a)-56(b), except substituting N-(N-tert-butoxycarbonyl-L-b-tert-butylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 3,4-dimethoxybenzoic acid for picolinic acid in step (b), the title compound was prepared as a white solid (73 mg, 51%). MS (ESI): 546.3 (M+H)⁺.

### Example 195

### Preparation of N-[N-(5-butylpicolinoyl)-L-b-tert-butylalanyl]-N′-[2-(N-cyclopropyl-N-cyclopropylmethylamino)thiazol-4-ylcarbonyl]hydrazide

Following the procedure of Example 56(a)-56(b), except substituting N-(N-tert-butoxycarbonyl-L-b-tert-butylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 5-butylpicolinic acid for picolinic acid in step (b), the title compound was prepared as a white solid (120 mg, 77%). MS (ESI): 543.2 (M+H)⁺.

### Example 196

### Preparation of N-[2-[N-cyclopropyl-N-(2-methylpropyl)amino]thiazol-4-ylcarbonyl]-N′-[N-(6-methylpicolinoyl)-L-b-tert-butylalanyl]hydrazide

Following the procedure of Example 56(a)-56(b), except substituting N-(N-tert-butoxycarbonyl-L-b-tert-butylalanyl)-N′-[2-[N-cyclopropyl-N-(2-methylpropyl)amino]thiazol-4-ylcarbonyl]hydrazide for N-[2-(N-cyclopropyl-N-cyclopropylmethylamino)thiazol-4-ylcarbonyl]-N′-(N-tert-butoxycarbonyl-L-leucinyl)hydrazide in step (a) and 6-methylpicolinic acid for picolinic acid in step (b), the title compound was prepared as a white solid (104 mg, 72%). MS (ESI): 501.3 (M+H)⁺.

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

We claim:

1. A compound of Formula I: 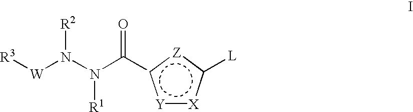
wherein:
L is C₂₋₆alkyl, Ar—C₀₋₆alkyl, Het—C₀₋₆alkyl, CH(R⁴)NR⁵R⁶, CH(R⁴)Ar, CH(R⁴)OAr′, or NR⁴R⁷;
Ar is phenyl or naphthyl;
Ar′ is phenyl or naphthyl;
Het is a stable 5- to 7-membered monocyclic or a stable 7- to 10-membered bicyclic heterocyclic ring, which is either saturated or unsaturated, and which consists of carbon atoms and from one to four heteroatoms selected from the group consisting of N, O and S, said heterocyclic ring being attached at any heteroatom or carbon atom which results in a stable structure, or any bicyclic group in which any of said monocyclic heterocyclic rings is fused to a benzene ring;
W is C(O), SO₂;
X, Y, and Z are independently N, O, S or CR¹⁰,
provided that at least two of X, Y and Z are heteroatoms and at least one of X, Y and Z is N, or that one of X, Y and Z is C═N, C═C or N═N and the other two are CR¹⁰ or N, further provided that at least two of X, Y and Z are N; [symbol]
indicates a single or double bond in the five-membered heterocycle;
R′, R¹, R², R⁵, R⁸, R⁹, R¹⁰, and R¹² are independently H, C₁₋₆alkyl, C₂₋₆alkenyl, Ar—C₀₋₆alkyl, or Het—C₀₋₆alkyl;
R³ is C₃₋₆alkyl, Ar, Het, CH(R¹¹)Ar, CH(R¹¹)OAr, NR¹¹R¹², CH(R¹¹)NR¹²R¹³; or 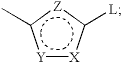
R⁴, R¹¹, and R¹⁵ are independently H, C₁₋₆alkyl, C₂₋₆alkenyl, C₃₋₆cycloalkyl-C₀₋₆-alkyl, Ar—C₀₋₆alkyl, or Het—C₀₋₆alkyl;
R⁷ is C₁₋₆alkyl, C₁₋₆alkenyl, C₃₋₆cycloalkyl-C₀₋₆-alkyl, Ar—C₀₋₆alkyl, or Het—C₀₋₆alkyl;
R⁶ and R¹³ are R¹⁴, R¹⁴C(O), R¹⁴C(S), R¹⁴OC(O), or R¹⁴OC(O)NR⁹CH(R¹⁵)(CO); and
R¹⁴ is C₁₋₆alkyl, C₂₋₆alkenyl, Ar—C₀₋₆alkyl, or Het—C₀₋₆alkyl.
and pharmaceutically acceptable salts, hydrates and solvates thereof.

2. A compound according to claim 1 wherein Ar is independently substituted by one or more moieties selected from the group consisting of: Ph—C₀₋₆alkyl, Het—C₀₋₆alkyl, C₁₋₆alkyl, C₁₋₆alkoxy, Ph—C₀₋₆alkoxy, Het—C₀₋₆alkoxy, OH, (CH₂)₁₋₆NR⁸R⁹, O(CH₂)₁₋₆NR⁸R⁹, CO₂R′, or halogen.

3. A compound according to claim 2 wherein Ph is independently substituted by one or more moieties selected from the group consisting of: C₁₋₆alkyl, C₁₋₆alkoxy, OH, (CH₂)₁₋₆NR⁸R⁹, O(CH₂)₁₋₆NR⁸R⁹, CO₂R′, and halogen.

4. A compound according to claim 2 wherein two C₁₋₆alkyl groups are combined to form a 5-7 membered ring, saturated or unsaturated, fused onto the Ar ring.

5. A compound according to claim 1 wherein Ar′ is independently substituted by one or more moieties selected from the group consisting of: Ph—C₀₋₆alkyl, Het—C₀₋₆alkyl, C₁₋₆alkyl, C₁₋₆alkoxy, Ph—C₀₋₆alkoxy, Het—C₀₋₆alkoxy, OH, (CH₂)₁₋₆NR⁸R⁹, O(CH₂)₁₋₆NR⁸R⁹, CO₂R′, or halogen.

6. A compound according to claim 5 wherein Ph is independently substituted by one or more moieties selected from the group consisting of: C₁₋₆alkyl, C₁₋₆alkoxy, OH, (CH₂)₁₋₆NR⁸R⁹, O(CH₂)₁₋₆NR⁸R⁹, CO₂R′, and halogen.

7. A compound according to claim 5 wherein two C₁₋₆alkyl groups are combined to form a 5-7 membered ring, saturated or unsaturated, fused onto the Ar′ ring.

8. A compound according to claim 1 wherein Het is independently substituted with one or two moieties selected from the group consisting of: Ph—C₀₋₆alkyl, Het—C₀₋₆ alkyl, C₁₋₆alkyl, C₁₋₆alkoxy, Ph—C₀₋₆alkoxy, Het—C₀₋₆alkoxy, OH, (CH₂)₁₋₆ NR⁸R⁹, O(CH₂)₁₋₆NR⁸R⁹, or CO₂R′.

9. A compound according to claim 8 wherein Ph is independently substituted by one or more moieties selected from the group consisting of: C₁₋₆alkyl, C₁₋₆alkoxy, OH, (CH₂)₁₋₆NR⁸R⁹, O(CH₂)₁₋₆NR⁸R⁹, CO₂R′, and halogen.

10. A compound according to claim 8 wherein two C₁₋₆alkyl groups are combined to form a 5-7 membered ring, saturated or unsaturated, fused onto the Het ring.

11. A compound according to claim 1 wherein Het is selected from the group consisting of the piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, triazolyl, tetrazolyl, pyridyl, pyrazinyl, pyridazinyl, pyrimidinyl, triazinyl, tetrazinyl, oxazolidinyl, oxazolinyl, oxazolyl, isothiazolyl, isoxazolyl, morpholinyl, thiazolidinyl, thiazolinyl, thiazolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, thiamorpholinyl sulfoxide, thiamorpholinyl sulfone, thiadiazolyl, and oxadiazolyl rings.

12. A compound according to claim 1 wherein R⁴ and R⁷ may be combined to form a 3-7 membered monocyclic or 7-10-membered bicyclic carbocyclic or heterocyclic ring.

13. A compound according to claim 12 wherein said 3-7 membered monocyclic or 7-10-membered bicyclic carbocyclic or heterocyclic ring is independently substituted with 1-4 moieties selected from the group consisting of: C₁₋₆alkyl, Ar—C₀₋₆alkyl, Het—C₀₋₆alkyl, C₁₋₆alkoxy, Ar—C₀₋₆alkoxy, Het—C₀₋₆alkoxy, OH, (CH₂)₁₋₆NR⁸R⁹, and O(CH₂)₁₋₆NR⁸R⁹.

14. A compound according to claim 1 wherein Z═N, X═S, and Y═CH.

15. A compound according to claim 14 wherein R³ is further defined as: 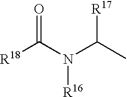
wherein:
R¹⁶ is H or C₁₋₆alkyl;
R¹⁷ is C₁₋₆alkyl, C₂₋₆alkenyl, or C₃₋₁₁cycloalkyl; and
R¹⁸ is C₃₋₆alkyl, OC₃₋₆alkyl, Ar, Het, O(CH₂)₀₋₃Ar, or O(CH₂)₀₋₃Het.

16. A compound according to claim 15 wherein R¹⁶ is H or Me.

17. A compound according to claim 15 wherein R¹⁷ is n-propyl, iso-propyl, iso-pentyl, tert-butylmethyl, cyclopropylmethyl, isobutyl, n-butyl, or allyl.

18. A compound according to claim 15 wherein R¹⁸ is selected from the group consisting of: 2-pyridinylmethoxy, 3-pyridinylmethoxy, 4-pyridinylmethoxy, ten-butoxy, 2-pyridinyl, 3-pyridinyl, 4-pyridinyl, 2-pyrazinyl, 4-tert-butoxycarbonylbenzyloxy, 4-carboxybenzyloxy, 3-tert-butoxycarbonylbenzyloxy, 3-carboxybenzyloxy, 2-methyl-3-pyridinylmethoxy, 6-methyl-3-pyridinylmethoxy, benzyloxy, 2-quinolino, 3-quinolino, 4-quinolino, 5-quinolino, 6-quinolino, 7-quinolino, 8-quinolino, 1-isoquinolino, 3-isoquinolino, piperidinyl, 4-methylpiperidinyl, 4-methylimidazol-5-yl, N-benzyl-pyrrolidinyl, N-methyl-pyrrolidinyl, 1-benzyl-5-methylimidazol-4-yl, 1-piperazinyl; 3-(2-pyridyl)benzyl, 2-methyl-3-pyridinyl, 2-methyl-4-pyridinyl, 6-methyl-3-pyridinyl, 4-dimethylaminobenzyloxy, 4-(4-morpholinomethyl)phenyl, 5-hydroxymethylimidazol-4-yl, 5-butyl-2-pyridinyl, 4-fluorophenyl, 3,4-difluorophenyl, 2-(1,8-naphthyridinyl), or 3,4-dimethoxyphenyl.

19. A compound according to claim 14 wherein L is selected from the group consisting of: 4-(cis-2,6-dimethyl)-4-morpholinyl, N-cyclopropylmethyl-N-(2-methylpropyl)amino, 4-methyl-1-naphthyl, N-methyl-N-(2-methylpropyl)amino, 1-naphthyl, 5-acenaphthyl, N-cyclopropyl-N-cyclopropylmethylamino, N,N-bis-(2-methylpropyl)amino, 1-(1,2,3,4-tetrahydroquinolino, N-cyclopropylmethyl-N-propylamino, N-(2-methylpropyl)-N-phenylamino, 2-methoxy-1-naphthyl, 2-benzyloxyphenyl, 2-benzyloxy-1-naphthyl, 9-phenanthrenyl, 9-anthracenyl, phenyl, 2-(4-tert-butoxycarbonyl)benzyloxyphenyl, 2-(4-carboxybenzyloxy)phenyl, N-cyclopropylamino, 8-quinolino, N,N-bis-(cyclopropylmethyl)amino, 4-(2,2-dimethylaminoethoxy)-1-naphthyl, or 1-(N-benzyloxycarbonylamino)-3-methylbutyl.

20. A compound according to claim 1 selected from the group consisting of:
N-[2-(cis-2,6-dimethyl-4-morpholino)thiazol-4-ylcarbonyl]-N′-[N-(4pyridinylmethoxycarbonyl)-L-leucinyl]hydrazide;
N-[2-[N-cyclopropylmethyl-N-(2-methylpropyl)amino]thiazol-4-ylcarbonyl]-N′-[N-(4-pyridinylmethoxycarbonyl)-L-leucinyl]hydrazide;
N-[2-(4-methyl-1-naphthyl)thiazol-4-ylcarbonyl]-N′-[N-(4-pyridinylmethoxycarbonyl)-L-leucinyl]hydrazide;
N-[2-[N-methyl-N-(2-methylpropyl)amino]thiazol-4-ylcarbonyl]-N′-[N-methyl-N-(4-pyridinylmethoxycarbonyl)-L-leucinyl]hydrazide;
N-[2-(1-naphthyl)thiazol-4-ylcarbonyl]-N′-[N-(3-pyridinylmethoxycarbonyl)-L-leucinyl]hydrazide;
N-[2-(1-naphthyl)thiazol-4-ylcarbonyl]-N′-[N-(2-pyridinylmethoxycarbonyl)-L-leucinyl]hydrazide;
N-[2-(5-acenaphthyl)thiazol-4-ylcarbonyl]-N′-[N-(4-pyridinylmethoxycarbonyl)-L-leucinyl]hydrazide;
N-[2-[N-cyclopropylmethyl-N-(2-methylpropyl)amino]thiazol-4-ylcarbonyl]-N′-[N-methyl-N-(4-pyridinylmethoxycarbonyl)-L-leucinyl]hydrazide;
N-[2-(N-cyclopropyl-N-cyclopropylmethylamino)thiazol-4-ylcarbonyl]-N′-[N-(4-pyridinylmethoxycarbonyl)-L-leucinyl]hydrazide;
N-[2-[N-cyclopropylmethyl-N-(2-methylpropyl)amino]thiazol-4-ylcarbonyl]-N′-[N-(3-pyridinylmethoxycarbonyl)-L-leucinyl]hydrazide;
N-[2-[N-cyclopropylmethyl-N-(2-methylpropyl)amino]thiazol-4-ylcarbonyl]-N′-[N-(2-pyridinylmethoxycarbonyl)-L-leucinyl]hydrazide;
N-[2-[N-cyclopropylmethyl-N-(2-methylpropyl)amino]thiazol-4-ylcarbonyl]-N′-[N-methyl-N-(3-pyridinylmethoxycarbonyl)-L-leucinyl]hydrazide;
N-[2-(N-cyclopropyl-N-cyclopropylmethylamino)thiazol-4-ylcarbonyl]-N′-[N-(3-pyridinylmethoxycarbonyl)-L-leucinyl]hydrazide;
N-[2-N-cyclopropyl-N-cyclopropylmethylamino)thiazol-4-ylcarbonyl]-N′-[N-methyl-N-(4-pyridinylmethoxycarbonyl)-L-leucinyl]hydrazide;
N-[2-[N,N-bis-(2-methylpropyl)amino]thiazol-4-ylcarbonyl]-N′-[N-(2-pyridinylmethoxycarbonyl)-L-leucinyl]hydrazide;
N-[N-(4-pyridinylmethoxycarbonyl)-L-leucinyl]-N′-[2-[1-(1,2,3,4-tetrahydroquinolino)]thiazol-4-ylcarbonyl]hydrazide;
N-[2-[N-methyl-N-(2-methylpropyl)amino]thiazol-4-ylcarbonyl]-N′-[4-methyl-2-(3-phenyl)phenylpent-4-enoyl]hydrazide;
N-[2-[N,N-bis-(2-methylpropyl)amino]thiazol-4-ylcarbonyl]-N′-[N-methyl-N-(3-pyridinylmethoxycarbonyl)-L-leucinyl]hydrazide;
N-[2-(N-cyclopropyl-N-cyclopropylmethylamino)thiazol-4-ylcarbonyl]-N′-[N-methyl-N-(3-pyridinylmethoxycarbonyl)-L-leucinyl]hydrazide;
N-[2-(N-cyclopropylmethyl-N-propylamino)thiazol-4-ylcarbonyl]-N′-[N-(3-pyridinylmethoxycarbonyl)-L-leucinyl]hydrazide;
N-[2-[N-methyl-N-(2-methylpropyl)amino]thiazol-4-ylcarbonyl]-N′-[4-methyl-2-(3-phenyl)phenylpentanoyl]hydrazide;
N-[N-(2-methylpropyl)-N-(3-phenylphenyl)carbamoyl]-N′-[2-(1-naphthyl)thiazol-4-ylcarbonyl]hydrazide;
N-[4-methyl-2-(3-phenyl)phenylpentanoyl]-N′-[2-(1-naphthyl)thiazol-4-ylcarbonyl]hydrazide;
N-[4-methyl-2-(3-phenyl)phenylpentanoyl]-N′-[2-[N-(2-methylpropyl)-N-phenylamino]thiazol-4-ylcarbonyl]hydrazide;
N-[2-(2-methoxy-1-naphthyl)thiazol-4-ylcarbonyl]-N′-[N-(4-pyridinylmethoxycarbonyl)-L-leucinyl]hydrazide;
N-[2-(2-benzyloxyphenyl)thiazol-4-ylcarbonyl]-N′-[4-methyl-2-(3-phenyl)phenylpentanoyl]hydrazide;
N-[2-(2-benzyloxy-1-naphthyl)thiazol-4-ylcarbonyl-N′-[N-(4-pyridinylmethoxycarbonyl)-L-leucinyl]hydrazide;
N-[2-[N,N-bis-(2-methylpropyl)aminothiazol-4-ylcarbonyl]-N′-[N-methyl-N-(2-pyridinylmethoxycarbonyl)-L-leucinyl]hydrazide;
N-[2-(9-phenanthrenyl)thiazol-4-ylcarbonyl]-N′-[N-(4-pyridinylmethoxycarbonyl)-L-leucinyl]hydrazide;
N-[2-(9-anthracenyl)thiazol-4-ylcarbonyl]-N′-[N-(4-pyridinylmethoxycarbonyl)-L-leucinyl]hydrazide;
N-[2-[N,N-bis-(2-methylpropyl)amino]thiazol-4-ylcarbonyl-N′-(-tert-butoxycarbonyl-L-leucinyl)ydrazide;
N-[2-[N,N-bis-(2-methylpropyl)amino]thiazol-4-ylcarbonyl]-N′-[N-(L-leucinyl)]hydrazide;
N-[2-(1-naphthyl)thiazol-4-ylcarbonyl]-N′-[N-methyl-N-(3-pyridinylmethoxycarbonyl)-L-leucinyl]hydrazide;
N-[2-[N,N-bis-(2-methylpropyl)amino]thiazol-4-ylcarbonyl]-N′-(N-picolinoyl-L-leucinyl)hydrazide;
N-[2-[N,N-bis-(2-methylpropyl)amino]thiazol-4-ylcarbonyl]-N′-[N-(2-pyrazinecarbonyl)-L-leucinyl]hydrazide;
N-[N,N-bis-(2-methylpropyl)carbamoyl]-N′-[2-[N-(2-methylpropyl)-N-phenylamino]thiazol-4-ylcarbonyl]hydrazide;
N-(2-phenylthiazol-4-ylcarbonyl)-N′-[N-(4-pyridinylmethoxycarbonyl)-L-leucinyl]hydrazide;
N-[2-[2-(4-tert-butoxycarbonyl)benzyloxyphenyl]thiazol-4-ylcarbonyl]-N′-[N-(4-pyridinylmethoxycarbonyl)-L-leucinyl]hydrazide;
N-[2-[2-(4-carboxybenzyloxy)phenyl]thiazol-4-ylcarbonyl]-N′-[N-(4-pyridinylmethoxycarbonyl)-L-leucinyl]hydrazide;
N-[N-(4-tert-butoxycarbonylbenzyloxycarbonyl)-L-leucinyl]-N′-[2-[N-(2-methylpropyl)-N-phenylamino]thiazol-4-ylcarbonyl]hydrazide;
N-[2-[N,N-bis-(2-methylpropyl)amino]thiazol-4-ylcarbonyl]-N′-[N-(4-tert-butoxycarbonylbenzyloxycarbonyl)-L-leucinyl]hydrazide;
N-[N-(4-carboxybenzyloxycarbonyl)-L-leucinyl]-N′-[2-[N-(2-methylpropyl)-N-phenylamino]thiazol-4-ylcarbonyl]hydrazide;
N-(N-benzyloxycarbonyl-L-leucinyl)-N′-[2-[2-(4-tert-butoxycarbonyl)benzyloxyphenyl]thiazol-4-ylcarbonyl]hydrazide;
N-(N-benzyloxycarbonyl-L-leucinyl)-N′-[2-[2-(4-carboxybenzyloxy)phenyl]thiazol-4-ylcarbonyl]hydrazide;
N-[N-(6-methyl-3-pyridinylmethoxycarbonyl)-L-leucinyl]-N′-[2-(1-naphthyl)thiazol-4-ylcarbonyl]hydrazide;
N-(N-benzyloxycarbonyl-L-leucinyl)-N′-[2-(N-cyclopropyl-N-cyclopropylmethylamino)thiazol-4-ylcarbonyl]hydrazide;
N-[2-(N-cyclopropyl-N-cyclopropylmethylamino)thiazol-4-ylcarbonyl]-N′-[N-(2-pyridinylmethoxycarbonyl)-L-leucinyl]hydrazide;
N-[2-(N-cyclopropyl-N-cyclopropylmethylamino)thiazol-4-ylcarbonyl]-N′-[N-(6-methyl-3-pyridinylmethoxycarbonyl)-L-leucinyl]hydrazide;
N-(N-tert-butoxycarbonyl-L-leucinyl)-N′-[2-(N-cyclopropyl-N-cyclopropylmethylamino)thiazol-4-ylcarbonyl]hydrazide;
N-[2-(N-cyclopropyl-N-cyclopropylmethylamino)thiazol-4-ylcarbonyl]-N′-[N-methyl-N-(2-pyridinylmethoxycarbonyl)-L-leucinyl]hydrazide;
N-[2-(2-benzyloxyphenyl)thiazol-4-ylcarbonyl]-N′-[N-(6-methyl-3-pyridinylmethoxycarbonyl)-L-leucinyl]hydrazide;
N-[2-(2-benzyloxyphenyl)thiazol-4-ylcarbonyl]-N′-[N-(2-methyl-3-pyridinylmethoxycarbonyl)-L-leucinyl]hydrazide;
N-[2-[N-methyl-N-(2-methylpropyl)amino]thiazol-4-ylcarbonyl]-N′-[N-(6-methyl-3-pyridinylmethoxycarbonyl)-L-leucinyl]hydrazide;
N-[2-[N-methyl-N-(2-methylpropyl)amino]thiazol-4-ylcarbonyl]-N′-[N-( )-L-leucinyl]hydrazide;
N-[2-(N-cyclopropyl-N-cyclopropylmethylamino)thiazol-4-ylcarbonyl]-N′-(N-picolinoyl-L-leucinyl) hydrazide;
N-[2-(N-cyclopropyl-N-cyclopropylmethylamino)thiazol-4-ylcarbonyl]-N′-[N-(2-methyl-3-pyridinylmethoxycarbonyl)-L-leucinyl]hydrazide;
N-[N-(3-tert-butoxycarbonylbenzyloxycarbonyl)-L-leucinyl]-N′-[2-(N-cyclopropyl-N-cyclopropylmethylamino)thiazol-4-ylcarbonyl]hydrazide;
N-[2-(1-naphthyl)thiazol-4-ylcarbonyl]-N′-[N-(8-quinolinoyl)-L-leucinyl]hydrazide;
N-[N-(2-methyl-3-pyridinylmethoxycarbonyl)-L-leucinyl]-N′-[2-(1-naphthyl)thiazol-4-ylcarbonyl]hydrazide;
N-[2-(1-naphthyl)thiazol-4-ylcarbonyl]-N′-(N-picolinoyl-L-leucinyl)hydrazide;
N-[N-(3-carboxybenzyloxycarbonyl)-L-leucinyl]-N′-[2-(N-cyclopropyl-N-cyclopropylmethylamino)thiazol-4-ylcarbonyl]hydrazide;
N-[2-(1-naphthyl)thiazol-4-ylcarbonyl]-N′-[N-(2-quinolinoyl)-L-leucinyl]hydrazide;
N-[2-(1-naphthyl)thiazol-4-ylcarbonyl]-N′-[N-(3-quinolinoyl)-L-leucinyl]hydrazide;
N-[2-(1-naphthyl)thiazol-4-ylcarbonyl]-N′-[N-(4-methylpiperidinecarbonyl)-L-leucinyl]hydrazide;
N-[2-(1-naphthyl)thiazol-4-ylcarbonyl]-N′-[N-(4-quinolinoyl)-L-leucinyl]hydrazide;
N-[2-(1-naphthyl)thiazol-4-ylcarbonyl]-N′-[N-(5-quinolinoyl)-L-leucinyl]hydrazide;
N-[2-(1-naphthyl)thiazol-4-ylcarbonyl]-N′-[N-(7-quinolinoyl)-L-leucinyl]hydrazide;
N-[2-(1-naphthyl)thiazol-4-ylcarbonyl]-N′-[N-(6-quinolinoyl)-L-leucinyl]hydrazide;
N-[N-(1-isoquinolinoyl)-L-leucinyl]-N′-[2-(1-naphthyl)thiazol-4-ylcarbonyl]hydrazide;
N-[N-(3-isoquinolinoyl)-L-leucinyl]-N′-[2-(1-naphthyl)thiazol-4-ylcarbonyl]hydrazide;
N-[N-(4-methylimidazol-5-ylcarbonyl)-L-leucinyl]-N′-[2-(1-naphthyl)thiazol-4-ylcarbonyl]hydrazide;
N-(N-benzyl-L-prolinyl-L-leucinyl)-N′-[2-(1-naphthyl)thiazol-4-ylcarbonyl]hydrazide;
N-[N-(1-benzyl-5-methylimidazol-4-ylcarbonyl)-L-leucinyl]-N′-[2-(1-naphthyl)thiazol-4-ylcarbonyl]hydrazide;
N-[N-(3-methylisonicotinoyl)-L-leucinyl]-N′-[2-(1-naphthyl)thiazol-4-ylcarbonyl]hydrazide;
N-[2-(N-cyclopropylamino)thiazol-4-ylcarbonyl]-N′-[N-(2-pyridinylmethoxycarbonyl)-L-leucinyl]hydrazide;
N-[4-methyl-2-(3-phenoxy)phenylpentanoyl]-N′-[2-(1-naphthyl)thiazol-4-ylcarbonyl]hydrazide;
N-[N-(2-benzoxazolyl)-L-leucinyl]-N′-[2-(1-naphthyl)thiazol-4-ylcarbonyl]hydrazide;
N-(N-benzyloxycarbonyl-L-leucinyl)-N′-[2-[N,N-bis-(2-methylpropyl)amino]oxazol-4-ylcarbonyl]hydrazide;
N-[2-[N-cyclopropyl-N-(2-methylpropyl)amino]thiazol-4-ylcarbonyl]-N′-[N-(2-pyridinylmethoxycarbonyl)-L-leucinyl]hydrazide;
N-[2-[N-cyclopropyl-N-(2-methylpropyl)amino]thiazol-4-ylcarbonyl]-N′-[N-methyl-N-(2-pyridinylmethoxycarbonyl)-L-leucinyl]hydrazide;
N-[2-(1-naphthyl)thiazol-4-ylcarbonyl]-N′-[N-(1-piperazinecarbonyl)-L-leucinyl]hydrazide;
N-[4-methyl-2-(4-phenoxy)phenylpentanoyl]-N′-[2-(1-naphthyl)thiazol-4-ylcarbonyl]hydrazide;
N-[2-[N-bis-(cyclopropylmethyl)amino]thiazol-4-ylcarbonyl]-N′-[N-(2-pyridinylmethoxycarbonyl)-L-leucinyl]hydrazide;
N-[2-(N-cyclopropyl-N-cyclopropylmethylamino)thiazol-4-ylcarbonyl]-N′-[N-(2-quinolinoyl)-L-leucinyl]hydrazide;
N-[N-(8-quinolinoyl)-L-leucinyl]-N′-[2-(8-quinolinyl)thiazol-4-ylcarbonyl]hydrazide;
N-(N-benzyloxycarbonyl-L-leucinyl)-N′-[2-(1-naphthyl)thiazol-4-ylcarbonyl]hydrazide;
N-[2-(N-cyclopropyl-N-cyclopropylmethylamino)thiazol-4-ylcarbonyl]-N′-[N-(3-quinolinoyl)-L-leucinyl]hydrazide;
N-[2-(N-cyclopropyl-N-cyclopropylmethylamino)thiazol-4-ylcarbonyl]-N′-[N-(3-isoquinolinoyl)-L-leucinyl]hydrazide;
N-[2-(N-cyclopropyl-N-cyclopropylmethylamino)thiazol-4-ylcarbonyl]-N′-[N-(6-quinolinoyl)-L-leucinyl]hydrazide;
N-[2-[N-bis-(cyclopropylmethyl)amino]thiazol-4-ylcarbonyl]-N′-[N-(2-methyl-3-pyridinylmethoxycarbonyl)-L-leucinyl]hydrazide;
N-(N-benzyloxycarbonyl-L-b-tert-butylalanyl)-N′-[2-(1-naphthyl)thiazol-4-ylcarbonyl]hydrazide;
N-(N-benzyloxycarbonyl-L-b-cyclopropylalanyl)-N′-[2-(1-naphthyl)thiazol-4-ylcarbonyl]hydrazide;
N-[2-(1-naphthyl)thiazol-4-ylcarbonyl]-N′-[N-[3-(2-pyridyl)phenylacetyl]-L-leucinyl]hydrazide;
N-[2-[N-bis-(cyclopropylmethyl)amino]thiazol-4-ylcarbonyl]-N′-(N-picolinyl-L-leucinyl) hydrazide;
N-(N-benzyloxycarbonyl-L-leucinyl)-N′-[2-[N-bis-(cyclopropylmethyl)amino]thiazol-4-ylcarbonyl]hydrazide;
N-[2-(N-cyclopropyl-N-cyclopropylmethylamino)thiazol-4-ylcarbonyl]-N′-[N-(6-methylnicotinoyl)-L-leucinyl]hydrazide;
N-[2-(N-cyclopropyl-N-cyclopropylmethylamino)thiazol-4-ylcarbonyl]-N′-[N-(2-methylnicotinoyl)-L-leucinyl]hydrazide;
N-[2-(N-cyclopropyl-N-cyclopropylmethylamino)thiazol-4-ylcarbonyl]-N′-[N-(3-methylisonicotinoyl)-L-leucinyl]hydrazide;
N-[2-(N-cyclopropyl-N-cyclopropylmethylamino)thiazol-4-ylcarbonyl]-N′-[N-(8-quinolinoyl)-L-leucinyl]hydrazide;
N-[2-[N-bis-(cyclopropylmethyl)amino]thiazol-4-ylcarbonyl]-N′-[N-(8-quinolinoyl)-L-leucinyl]hydrazide;
N-[2-[N-bis-(cyclopropylmethyl)amino]thiazol-4-ylcarbonyl]-N′-[N-(3-isoquinolinoyl)-L-leucinyl]hydrazide;
N-[2-[4-(2,2-dimethylaminoethoxy)-1-naphthyl]thiazol-4-ylcarbonyl]-N′-[N-(8-quinolinoyl)-L-leucinyl]hydrazide;
N-[2-(N-cyclopropyl-N-cyclopropylmethylamino)thiazol-4-ylcarbonyl]-N′-[N-(7-quinolinoyl)-L-leucinyl]hydrazide;
N-[2-[N-bis-(cyclopropylmethyl)amino]thiazol-4-ylcarbonyl]-N′-[N-(6-methylnicotinoyl)-L-leucinyl]hydrazide;
N-[2-[N-bis-(cyclopropylmethyl)amino]thiazol-4-ylcarbonyl]-N′-(N-methyl-L-prolinyl-L-leucinyl) hydrazide;
N-(N-benzyloxycarbonyl-L-norvalinyl)-N′-[2-(1-naphthyl)thiazol-4-ylcarbonyl]hydrazide;
N-(N-benzyloxycarbonyl-L-isoleucinyl)-N′-[2-(1-naphthyl)thiazol-4-ylcarbonyl]hydrazide;
N-[N-(4-dimethylaminomethylbenzoyl)-L-leucinyl]-N′-[2-(1-naphthyl)thiazol-4-ylcarbonyl]hydrazide;
N-(N-benzyloxycarbonyl-L-norleucinyl)-N′-[2-(1-naphthyl)thiazol-4-ylcarbonyl]hydrazide;
N-[N-(4-dimethylaminomethylbenzyloxycarbonyl)-L-leucinyl]-N′-[2-(1-naphthyl)thiazol-4-ylcarbonyl]hydrazide;
N-(N-benzyloxycarbonyl-L-norvalinyl)-N′-[2-(2-benzyloxyphenyl)thiazol-4-ylcarbonyl]hydrazide;
N-[2-(N-cyclopropyl-N-cyclopropylmethylamino)thiazol-4-ylcarbonyl]-N′-[N-(4-methylimidazol-5-ylcarbonyl)-L-leucinyl]hydrazide;
N-[N-[4-(4-morpholinomethyl)benzoyl]-L-leucinyl]-N′-[2-(1-naphthyl)thiazol-4-ylcarbonyl]hydrazide;
N-[N-(2-methylnicotinoyl)-L-leucinyl]-N′-[2-(1-naphthyl)thiazol-4-ylcarbonyl]hydrazide;
N-[N-(6-methylnicotinoyl)-L-leucinyl]-N′-[2-(1-naphthyl)thiazol-4-ylcarbonyl]hydrazide;
N-(N-b-tert-butoxycarbonyl-L-tert-butylalanyl)-N′-[2-(N-cyclopropyl-N-cyclopropylmethylamino)thiazol-4-ylcarbonyl]hydrazide;
N-[2-(N-cyclopropyl-N-cyclopropylmethylamino)thiazol-4-ylcarbonyl]-N′-[N-(8-quinolinoyl)-L-b-tert-butylalanyl]hydrazide;
N-[N-(4-methylimidazol-5-ylcarbonyl)-L-allylglycinyl]-N′-[2-(1-naphthyl)thiazol-4-ylcarbonyl]hydrazide;
N-[N-(4-methylimidazol-5-ylcarbonyl)-L-b-tert-butylalanyl]-N′-[2-(1-naphthyl)thiazol-4-ylcarbonyl]hydrazide;
N-[2-(N-cyclopropyl-N-cyclopropylmethylamino)thiazol-4-ylcarbonyl]-N′-[N-(4-methylimidazol-5-ylcarbonyl)-L-b-tert-butylalanyl]hydrazide;
N-[2-(1-naphthyl)thiazol-4-ylcarbonyl]-N′-(N-picolinoyl-L-b-tert-butylalanyl)hydrazide;
N-[2-(1-naphthyl)thiazol-4-ylcarbonyl]-N′-[N-(8-quinolinoyl)-L-b-tert-butylalanyl]hydrazide;
N-[2-(1-naphthyl)thiazol-4-ylcarbonyl]-N′-(N-picolinoyl-L-allylglycinyl)hydrazide;
N-[2-(1-naphthyl)thiazol-4-ylcarbonyl]-N′-(N-picolinoyl-L-b-cyclopropylalanyl)hydrazide;
N-[N-(6-methylnicotinoyl)-L-b-cyclopropylalanyl]-N′-[2-(1-naphthyl)thiazol-4-ylcarbonyl]hydrazide;
N-[N-(4-methylimidazol-5-ylcarbonyl)-L-b-cyclopropylalanyl]-N′-[2-(1-naphthyl)thiazol-4-ylcarbonyl]hydrazide;
N-[2-(1-naphthyl)thiazol-4-ylcarbonyl]-N′-[N-(8-quinolinoyl)-L-b-cyclopropylalanyl]hydrazide;
N-[N-(6-methylnicotinoyl)-L-b-tert-butylalanyl]-N′-[2-(1-naphthyl)thiazol-4-ylcarbonyl]hydrazide;
N-[2-(N-cyclopropyl-N-cyclopropylmethylamino)thiazol-4-ylcarbonyl]-N′-(N-picolinoyl-L-b-tert-butylalanyl)hydrazide;
N-[2-(N-cyclopropyl-N-cyclopropylmethylamino)thiazol-4-ylcarbonyl]-N′-[N-(3-isoquinolinoyl)-L-b-tert-butylalanyl]hydrazide;
N-(N-tert-butoxycarbonyl-L-b-cyclopropylalanyl)-N′-[2-(N-cyclopropyl-N-cyclopropylmethylamino)thiazol-4-ylcarbonyl]hydrazide;
N-[2-(N-cyclopropylmethyl-N-propylamino)thiazol-4-ylcarbonyl]-N′-[N-(6-methyl-3-pyridinylmethoxycarbonyl)-L-leucinyl]hydrazide;
N-[N-(6-methylnicotinoyl)-L-allylglycinyl]-N′-[2-(1-naphthyl)thiazol-4-ylcarbonyl]hydrazide;
N-[2-(1-naphthyl)thiazol-4-ylcarbonyl-N′-[N-(8-quinolinoyl)-L-allylglycinyl]hydrazide;
N-[2-(1-naphthyl)thiazol-4-ylcarbonyl]-N′-[N-(2-quinolinoyl)-L-b-cyclopropylalanyl]hydrazide;
N-[N-(3-isoquinolinoyl)-L-b-cyclopropylalanyl]-N′[-2-(1-naphthyl)thiazol-4-ylcarbonyl]hydrazide;
N-[N-(1-isoquinolinoyl)-L-b-cyclopropylalanyl]-N′-[2-(1-naphthyl)thiazol-4-ylcarbonyl]hydrazide;
N-[2-(1-naphthyl)thiazol-4-ylcarbonyl]-N′-[N-(7-quinolinoyl)-L-b-cyclopropylalanyl]hydrazide;
N-[2-(N-cyclopropyl-N-cyclopropylmethylamino)thiazol-4-ylcarbonyl]-N′-[N-(8-quinolinoyl)-L-b-cyclopropylalanyl]hydrazide;
N-[2-(N-cyclopropyl-N-cyclopropylmethylamino)thiazol-4-carbonyl]-N′-[N-(4-methylimidazol-5-ylcarbonyl)-L-b-cyclopropylalanyl]hydrazide;
N-[2-(N-cyclopropyl-N-cyclopropylmethylamino)thiazol-4-ylcarbonyl]-N′-[N-(3-isoquinolinoyl)-L-b-cyclopropylalanyl]hydrazide;
N-[2-(N-cyclopropyl-N-cyclopropylmethylamino)thiazol-4-ylcarbonyl]-N′-[N-(6-methylnicotinoyl)-L-b-cyclopropylalanyl]hydrazide;
N-[N-(4-methylimidazol-5-ylcarbonyl)-L-norleucinyl]-N′-[2-(1-naphthyl)thiazol-4-ylcarbonyl]hydrazide;
N-[2-(1-naphthyl)thiazol-4-ylcarbonyl]-N′-(N-picolinoyl-L-norleucinyl)hydrazide;
N-[2-(1-naphthyl)thiazol-4-ylcarbonyl]-N′-[N-(8-quinolinoyl)-L-norleucinyl]hydrazide;
N-[2-(N-cyclopropyl-N-cyclopropylmethylamino)thiazol-4-ylcarbonyl]-N′-[N-(2-quinolinoyl)-L-b-cyclopropylalanyl]hydrazide;
N-[2-(N-cyclopropyl-N-cyclopropylmethylamino)thiazol-4-ylcarbonyl]-N′-[N-(1-isoquinolinoyl)-L-b-cyclopropylalanyl]hydrazide;
N-[2-[N-cyclopropyl-N-(2-methylpropyl)amino]thiazol-4-ylcarbonyl]-N′-[N-(6-methyl-3-pyridinylmethoxycarbonyl)-L-leucinyl]hydrazide;
N-(N-tert-butoxycarbonyl-L-leucinyl)-N′-[2-[N-cyclopropyl-N-(2-methylpropyl)amino]thiazol-4-ylcarbonyl]hydrazide;
N-[2-(1-naphthyl)thiazol-4-ylcarbonyl]-N′-[N-(7-quinolinoyl)-L-b-tert-butylalanyl]hydrazide;
N-[2-(1-naphthyl)thiazol-4-ylcarbonyl]-N′-[N-(2-quinolinoyl)-L-b-tert-butylalanyl]hydrazide;
N-[N-(1-isoquinolinoyl)-L-b-tert-butylalanyl]-N′-[2-(-naphthyl)thiazol-4-ylcarbonyl]hydrazide;
N-[N-(3-isoquinolinoyl)-L-b-tert-butylalanyl]-N′-[2-(1-naphthyl)thiazol-4-ylcarbonyl]hydrazide;
N-[N-(6-methylnicotinoyl)-L-norleucinyl]-N′-[2-(1-naphthyl)thiazol-4-ylcarbonyl]hydrazide;
N-[2-(1-naphthyl)thiazol-4-ylcarbonyl]-N′-[N-(7-quinolinoyl)-L-norleucinyl]hydrazide;
N-[2-(1-naphthyl)thiazol-4-ylcarbonyl]-N′-[N-(2-quinolinoyl)-L-norleucinyl]hydrazide;
N-[N-(1-isoquinolinoyl)-L-norleucinyl]-N′-[2-(1-naphthyl)thiazol-4-ylcarbonyl]hydrazide;
N-[N-(3-isoquinolinoyl)-L-norleucinyl]-N′-[2-(1-naphthyl)thiazol-4-ylcarbonyl]hydrazide;
N-[2-(N-cyclopropyl-N-cyclopropylmethylamino)thiazol-4-ylcarbonyl]-N′-[N-(5-hydroxymethylimidazol-4-ylcarbonyl)-L-b-cyclopropylalanyl]hydrazide;
N-[2-[N-cyclopropyl-N-(2-methylpropyl)amino]thiazol-4-ylcarbonyl]-N′-[N-(8-quinolinoyl)-L-b-cyclopropylalanyl]hydrazide;
N-[2-(N-cyclopropyl-N-cyclopropylmethylamino)thiazol-4-ylcarbonyl]-N′-(N-(6-methylnicotinoyl)-L-b-tert-butylalanyl]hydrazide;
N-[2-[N-cyclopropyl-N-(2-methylpropyl)amino]thiazol-4-ylcarbonyl]-N′-[N-(4-methylimidazol-5-ylcarbonyl)-L-b-cyclopropylalanyl]hydrazide;
N-[2-[N-cyclopropyl-N-(2-methylpropyl)amino]thiazol-4-ylcarbonyl]-N′-[N-(2-quinolinoyl)-L-b-cyclopropylalanyl]hydrazide;
N-[2-[N-cyclopropyl-N-(2-methylpropyl)amino]thiazol-4-ylcarbonyl]-N′-[N-(6-methylnicotinoyl)-L-b-cyclopropylalanyl]hydrazide;
N-[2-(1-naphthyl)thiazol-4-ylcarbonyl]-N′-[N-(8-quinolinoyl)glycinyl]hydrazide;
N-[2-(1-naphthyl)thiazol-4-ylcarbonyl]-N′-[N-(8-quinolinoyl)-L-norvalinyl]hydrazide;
N-[2-(1-naphthyl)thiazol-4-ylcarbonyl]-N′-[N-(2-quinolinoyl)-L-norvalinyl]hydrazide;
N-[2-(1-naphthyl)thiazol-4-ylcarbonyl]-N′-(N-picolinoyl-L-norvalinyl]hydrazide;
N-[2-(1-naphthyl)thiazol-4-ylcarbonyl]-N′-[N-(6-methylnicotinoyl)-L-norvalinyl]hydrazide;
N-[2-(1-naphthyl)thiazol-4-ylcarbonyl]-N′-[N-(4-methylimidazol-5-ylcarbonyl)-L-norvalinyl]hydrazide;
N-[2-(1-naphthyl)thiazol-4-ylcarbonyl]-N′-[N-(1-isoquinolinoyl)-L-norvalinyl]hydrazide;
N-[2-(1-naphthyl)thiazol-4-ylcarbonyl]-N′-[N-(3-isoquinolinoyl)-L-norvalinyl]hydrazide; (1S, 1′S)-N,N′-bis-[4-[1-(N-benzyloxycarbonylamino)-3-methylbutyl]thiazol-2-ylcarbonyl]hydrazide;
N-[2-[N-cyclopropyl-N-(2-methylpropyl)amino]thiazol-4-ylcarbonyl]-N′-[N-(6-methylnicotinoyl)-L-b-tert-butylalanyl]hydrazide;
N-[2-[N-cyclopropyl-N-(2-methylpropyl)amino]thiazol-4-ylcarbonyl]-N-[N-(4-methylimidazol-5-ylcarbonyl)-L-b-tert-butylalanyl]hydrazide;
N-[2-(N-cyclopropyl-N-cyclopropylmethylamino)thiazol-4-ylcarbonyl]-N′-[N-(1-isoquinolinoyl)-L-b-tert-butylalanyl]hydrazide;
N-[N-(5-butylpicolinoyl)-L-b-tert-butylalanyl]-N′-[2-(N-cyclopropyl-N-cyclopropylmethylamino)thiazol-4-ylcarbonyl]hydrazide;
N-[2-(N-cyclopropyl-N-cyclopropylmethylamino)thiazol-4-ylcarbonyl]-N′-[N-(6-methylpicolinoyl)-L-b-tert-butylalanyl]hydrazide;
N-[2-(N-cyclopropyl-N-cyclopropylmethylamino)thiazol-4-ylcarbonyl]-N′-[N-(4-fluorobenzoyl)-L-leucinyl]hydrazide;
N-[N-(4-fluorobenzoyl)-L-leucinyl]-N′-[2-(1-naphthyl)thiazol-4-ylcarbonyl]hydrazide;
N-[2-(1-naphthyl)thiazol-4-ylcarbonyl]-N′-[N-(2-pyridinylmethoxycarbonyl)-L-b-tert-butylalanyl]hydrazide;
N-[N-(2-methyl-3-pyridinylmethoxycarbonyl)-L-b-tert-butylalanyl]-N′-[2-(1-naphthyl)thiazol-4-ylcarbonyl]hydrazide;
N-[2-(1-naphthyl)thiazol-4-ylcarbonyl]-N′-[N-(2-pyridinylmethoxycarbonyl)-L-b-cyclopropylalanyl]hydrazide;
N-[N-(2-methyl-3-pyridinylmethoxycarbonyl)-L-b-cyclopropylalanyl]-N′-[2-(1-naphthyl)thiazol-4-ylcarbonyl]hydrazide;
N-[N-(6-methyl-3-pyridinylmethoxycarbonyl)-L-b-cyclopropylalanyl]-N′-[2-(1-naphthyl)thiazol-4-ylcarbonyl]hydrazide;
N-[N-(6-methyl-3-pyridinylmethoxycarbonyl)-L-b-tert-butylalanyl]-N′-[2-(1-naphthyl)thiazol-4-ylcarbonyl]hydrazide;
N,N′-bis-[2-(1-naphthyl)thiazol-4-ylcarbonyl]hydrazide;
N-[2-(N-cyclopropyl-N-cyclopropylmethylamino)thiazol-4-ylcarbonyl)-N-[N-[2-(1,8-naphthyridinoyl)]-L-b-cyclopropylalanyl]hydrazide;
N-[2-[N-cyclopropyl-N-(2-methylpropyl)amino]thiazol-4-ylcarbonyl]-N′-[N-(3,4-difluorobenzoyl)-L-b-cyclopropylalanyl]hydrazide;
N-(2-[N-cyclopropyl-N-(2-methylpropyl)amino]thiazol-4-ylcarbonyl]-N′-[N-(4-flluorobenzoyl)-L-leucinyl]hydrazide;
N-[N-(5-butylpicolinoyl)-L-leucinyl]-N′-[2-(N-cyclopropyl-N-cyclopropylmethylamino)thiazol-4-ylcarbonyl]hydrazide;
N-[2-[N-cyclopropyl-N-(2-methylpropyl)amino]thiazol-4-ylcarbonyl]-N′-[N-(3,4-dimethoxybenzoyl)-L-leucinyl]hydrazide;
N-[2-[N-cyclopropyl-N-(2-methylpropyl)amino]thiazol-4-ylcarbonyl]-N′-[N-(3,4-difluorobenzoyl)-L-b-tert-butylalanyl]hydrazide;
N-[2-[N-cyclopropyl-N-(2-methylpropyl)amino]thiazol-4-ylcarbonyl]-N′-[N-(3,4-dimethoxybenzoyl)-L-b-tert-butylalanyl]hydrazide;
N-[N-(5-butylpicolinoyl)-L-b-tert-butylalanyl]-N′-[2-(N-cyclopropyl-N-cyclopropylmethylamino)thiazol-4-ylcarbonyl]hydrazide; and
N-[2-[N-cyclopropyl-N-(2-methylpropyl)amino]thiazol-4-ylcarbonyl]-N′-[N-(6-methylpicolinoyl)-L-b-tert-butylalanyl]hydrazide.

21. A compound according to claim 20 which is selected from the group consisting of:
N-[2-[N-cyclopropylmethyl-N-(2-methylpropyl)amino]thiazol-4-ylcarbonyl]-N′-[N-(4-pyridinylmethoxycarbonyl)-L-leucinyl]hydrazide;
N-[2-(4-methyl-1-naphthyl)thiazol-4-ylcarbonyl]-N′-[N-(4-pyridinylmethoxycarbonyl)-L-leucinyl]hydrazide;
N-[2-[N-methyl-N-(2-methylpropyl)amino]thiazol-4-ylcarbonyl]-N′-[N-methyl-N-(4-pyridinylmethoxycarbonyl)-L-leucinyl]hydrazide;
N-[2-(1-naphthyl)thiazol-4-ylcarbonyl]-N′-[N-(3-pyridinylmethoxycarbonyl)-L-leucinyl]hydrazide;
N-[2-(1-naphthyl)thiazol-4-ylcarbonyl]-N′-[N-(2-pyridinylmethoxycarbonyl)-L-leucinyl]hydrazide;
N-[2-(5-acenaphthyl)thiazol-4-ylcarbonyl)-N′-[N-(4-pyridinylmethoxycarbonyl)-L-leucinyl]hydrazide;
N-[2-[N-cyclopropylmethyl-N-(2-methylpropyl)amino]thiazol-4-ylcarbonyl]-N′-[N-methyl-N-(4-pyridinylmethoxycarbonyl)-L-leucinyl]hydrazide;
N-[2-(N-cyclopropyl-N-cyclopropylmethylamino)thiazol-4-ylcarbonyl]-N′-[N-(4-pyridinylmethoxycarbonyl)-L-leucinyl]hydrazide;
N-[2-[N-cyclopropylmethyl-N-(2-methylpropyl)amino]thiazol-4-ylcarbonyl]-N′-[N-(3-pyridinylmethoxycarbonyl)-L-leucinyl]hydrazide;
N-[2-[N-cyclopropylmethyl-N-(2-methylpropyl)amino]thiazol-4-ylcarbonyl]-N′-[N-(2-pyridinylmethoxycarbonyl)-L-leucinyl]hydrazide;
N-[2-[N-cyclopropylmethyl-N-(2-methylpropyl)amino]thiazol-4-ylcarbonyl]-N′-[N-methyl-N-(3-pyridinylmethoxycarbonyl)-L-leucinyl]hydrazide;
N-[2-(N-cyclopropyl-N-cyclopropylmethylamino)thiazol-4-ylcarbonyl]-N′-[N-(3-pyridinylmethoxycarbonyl)-L-leucinyl]hydrazide;
N-[2-(N-cyclopropyl-N-cyclopropylmethylamino)thiazol-4-ylcarbonyl]-N′-[N-methyl-N-(4-pyridinylmethoxycarbonyl)-L-leucinyl]hydrazide;
N-[2-[N,N-bis-(2-methylpropyl)amino]thiazol-4-ylcarbonyl]-N′-[N-(2-pyridinylmethoxycarbonyl)-L-leucinyl]hydrazide;
N-[N-(4-pyridinylmethoxycarbonyl)-L-leucinyl]-N′-[2-[1-(1,2,3,4-tetrahydroquinolino)]thiazol-4-ylcarbonyl]hydrazide;
N-[2-[N,N-bis-(2-methylpropyl)amino]thiazol-4-ylcarbonyl]-N′-[N-methyl-N-(3-pyridinylmethoxycarbonyl)-L-leucinyl]hydrazide;
N-[2-(N-cyclopropyl-N-cyclopropylmethylamino)thiazol-4-ylcarbonyl]-N′-[N-methyl-N-(3-pyridinylmethoxycarbonyl)-L-leucinyl]hydrazide;
N-[2-(N-cyclopropylmethyl-N-propylamino)thiazol-4-ylcarbonyl]-N′-[N-(3-pyridinylmethoxycarbonyl)-L-leucinyl]hydrazide;
N-[2-(2-benzyloxy-1-naphthyl)thiazol-4-ylcarbonyl]-N′-[N-(4-pyridinylmethoxycarbonyl)-L-leucinyl]hydrazide;
N-[2-[N,N-bis-(2-methylpropyl)amino]thiazol-4-ylcarbonyl]-N′-[N-methyl-N-(2-pyridinylmethoxycarbonyl)-L-leucinyl]hydrazide;
N-[2-(9-phenanthrenyl)thiazol-4-ylcarbonyl]-N′-[N-(4-pyridinylmethoxycarbonyl)-L-leucinyl]hydrazide;
N-[2-[N,N-bis-(2-methylpropyl)amino]thiazol-4-ylcarbonyl]-N′-(-tert-butoxycarbonyl-L-leucinyl)ydrazide;
N-[2-[N,N-bis-(2-methylpropyl)amino]thiazol-4-ylcarbonyl]-N′-(N-picolinoyl-L-leucinyl)hydrazide;
N-[2-[N,N-bis-(2-methylpropyl)amino]thiazol-4-ylcarbonyl]-N′-[N-(2-pyrazinecarbonyl)-L-leucinyl]hydrazide;
N-[2-[2-(4-tert-butoxycarbonyl)benzyloxyphenyl]thiazol-4-ylcarbonyl]-N′-[N-(4-pyridinylmethoxycarbonyl)-L-leucinyl]hydrazide;
N-[2-[2-(4-carboxybenzyloxy)phenyl]thiazol-4-ylcarbonyl]-N′-[N-(4-pyridinylmethoxycarbonyl)-L-leucinyl]hydrazide;
N-[N-(4-tert-butoxycarbonylbenzyloxycarbonyl)-L-leucinyl]-N′-[2-[N-(2-methylpropyl)-N-phenylamino]thiazol-4-ylcarbonyl]hydrazide;
N-[2-[N,N-bis-(2-methylpropyl)amino]thiazol-4-ylcarbonyl]-N′-[N-(4-tert-butoxycarbonylbenzyloxycarbonyl)-L-leucinyl]hydrazide;
N-[N-(4-carboxybenzyloxycarbonyl)-L-leucinyl]-N′-[2-[N-(2-methylpropyl)-N-phenylamino]thiazol-4-ylcarbonyl]hydrazide;
N-[N-(6-methyl-3-pyridinylmethoxycarbonyl)-L-leucinyl]-N′-[2-(1-naphthyl)thiazol-4-ylcarbonyl]hydrazide;
N-(N-benzyloxycarbonyl-L-leucinyl)-N′-[2-(N-cyclopropyl-N-cyclopropylmethylamino)thiazol-4-ylcarbonyl]hydrazide;
N-[2-(N-cyclopropyl-N-cyclopropylmethylamino)thiazol-4-ylcarbonyl-N′-[N-(2-pyridinylmethoxycarbonyl)-L-leucinyl]hydrazide;
N-[2-(N-cyclopropyl-N-cyclopropylmethylamino)thiazol-4-ylcarbonyl]-N′-[N-(6-methyl-3-pyridinylmethoxycarbonyl)-L-leucinyl]hydrazide;
N-(N-tert-butoxycarbonyl-L-leucinyl)-N′-[2-(N-cyclopropyl-N-cyclopropylmethylamino)thiazol-4-ylcarbonyl]hydrazide;
N-[2-(N-cyclopropyl-N-cyclopropylmethylamino)thiazol-4-ylcarbonyl]-N′-[N-methyl-N-(2-pyridinylmethoxycarbonyl)-L-leucinyl]hydrazide;
N-[2-(2-benzyloxyphenyl)thiazol-4-ylcarbonyl]-N′-[N-(6-methyl-3-pyridinylmethoxycarbonyl)-L-leucinyl]hydrazide;
N-[2-(2-benzyloxyphenyl)thiazol-4-ylcarbonyl]-N′-[N-(2-methyl-3-pyridinylmethoxycarbonyl)-L-leucinyl]hydrazide;
N-[2-[N-methyl-N-(2-methylpropyl)amino]thiazol-4-ylcarbonyl]-N′-[N-(6-methyl-3-pyridinylmethoxycarbonyl)-L-leucinyl]hydrazide;
N-[2-[N-methyl-N-(2-methylpropyl)amino]thiazol-4-ylcarbonyl]-N′-[N-( )-L-leucinyl]hydrazide;
N-[2-(N-cyclopropyl-N-cyclopropylmethylamino)thiazol-4-ylcarbonyl]-N′-(N-picolinoyl-L-leucinyl)hydrazide;
N-[2-(N-cyclopropyl-N-cyclopropylmethylamino)thiazol-4-ylcarbonyl]-N′-[N-(2-methyl-3-pyridinylmethoxycarbonyl)-L-leucinyl]hydrazide;
N-[N-(3-tert-butoxycarbonylbenzyloxycarbonyl)-L-leucinyl]-N′-[2-(N-cyclopropyl-N-cyclopropylmethylamino)thiazol-4-ylcarbonyl]hydrazide;
N-[2-(1-naphthyl)thiazol-4-ylcarbonyl]-N′-[N-(8-quinolinoyl)-L-leucinyl]hydrazide;
N-[N-(2-methyl-3-pyridinylmethoxycarbonyl)-L-leucinyl]-N′-[2-(1-naphthyl)thiazol-4-ylcarbonyl]hydrazide;
N-[2-(1-naphthyl)thiazol-4-ylcarbonyl]-N′-(N-picolinoyl-L-leucinyl)hydrazide;
N-[N-(3-carboxybenzyloxycarbonyl)-L-leucinyl]-N′-[2-(N-cyclopropyl-N-cyclopropylmethylamino)thiazol-4-carbonyl]hydrazide;
N-[2-(1-naphthyl)thiazol-4-ylcarbonyl]-N′-[N-(2-quinolinoyl)-L-leucinyl]hydrazide;
N-[2-(1-naphthyl)thiazol-4-ylcarbonyl]-N′-[N-(3-quinolinoyl)-L-leucinyl]hydrazide;
N-[2-(1-naphthyl)thiazol-4-ylcarbonyl]-N′-[N-(4-methylpiperidinecarbonyl)-L-leucinyl]hydrazide;
N-[2-(1-naphthyl)thiazol-4-ylcarbonyl]-N′-[N-(4-quinolinoyl)-L-leucinyl]hydrazide;
N-[2-(1-naphthyl)thiazol-4-ylcarbonyl]-N′-[N-(5-quinolinoyl)-L-leucinyl]hydrazide;
N-[2-(1-naphthyl)thiazol-4-ylcarbonyl]-N′-[N-(7-quinolinoyl)-L-leucinyl]hydrazide;
N-[2-(1-naphthyl)thiazol-4-ylcarbonyl]-N′-[N-(6-quinolinoyl)-L-leucinyl]hydrazide;
N-[N-(1-isoquinolinoyl)-L-leucinyl]-N′-[2-(1-naphthyl)thiazol-4-ylcarbonyl]hydrazide;
N-[N-(3-isoquinolinoyl)-L-leucinyl]-N′-[2-(1-naphthyl)thiazol-4-ylcarbonyl]hydrazide;
N-[N-(4-methylimidazol-5-ylcarbonyl)-L-leucinyl]-N′-[2-(1-naphthyl)thiazol-4-ylcarbonyl]hydrazide;
N-[N-(1-benzyl-5-methylimidazol-4-ylcarbonyl)-L-leucinyl]-N′-[2-(1-naphthyl)thiazol-4-ylcarbonyl]hydrazide;
N-[N-(3-methylisonicotinoyl)-L-leucinyl]-N′-[2-(1-naphthyl)thiazol-4-ylcarbonyl]hydrazide;
N-(N-benzyloxycarbonyl-L-leucinyl)-N′-[2-[N,N-bis-(2-methylpropyl)amino]oxazol-4-ylcarbonyl]hydrazide;
N-[2-[N-cyclopropyl-N-(2-methylpropyl)amino]thiazol-4-ylcarbonyl]-N′-[N-(2-pyridinylmethoxycarbonyl)-L-leucinyl]hydrazide;
N-[2-[N-cyclopropyl-N-(2-methylpropyl)amino]thiazol-4-ylcarbonyl]-N′-[N-methyl-N-(2-pyridinylmethoxycarbonyl)-L-leucinyl]hydrazide;
N-[2-(1-naphthyl)thiazol-4-ylcarbonyl]-N′-[N-(1-piperazinecarbonyl)-L-leucinyl]hydrazide;
N-[2-[N-bis-(cyclopropylmethyl)amino]thiazol-4-ylcarbonyl]-N′-[N-(2-pyridinylmethoxycarbonyl)-L-leucinyl]hydrazide;
N-[2-(N-cyclopropyl-N-cyclopropylmethylamino)thiazol-4-ylcarbonyl]-N′-[N-(2-quinolinoyl)-L-leucinyl]hydrazide;
N-[N-(8-quinolinoyl)-L-leucinyl]-N′-[2-(8-quinolinyl)thiazol-4-ylcarbonyl]hydrazide;
N-(N-benzyloxycarbonyl-L-leucinyl)-N′-[2-(1-naphthyl)thiazol-4-ylcarbonyl]hydrazide;
N-[2-(N-cyclopropyl-N-cyclopropylmethylamino)thiazol-4-ylcarbonyl]-N′-[N-(3-quinolinoyl)-L-leucinyl]hydrazide;
N-[2-(N-cyclopropyl-N-cyclopropylmethylamino)thiazol-4-ylcarbonyl]-N′-[N-(3-isoquinolinoyl)-L-leucinyl]hydrazide;
N-[2-(N-cyclopropyl-N-cyclopropylmethylamino)thiazol-4-ylcarbonyl]-N′-[N-(6-quinolinoyl)-L-leucinyl]hydrazide;
N-[2-[N-bis-(cyclopropylmethyl)amino]thiazol-4-ylcarbonyl]-N′-[N-(2-methyl-3-pyridinylmethoxycarbonyl)-L-leucinyl]hydrazide;
N-(N-benzyloxycarbonyl-L-b-tert-butylalanyl)-N′-[2-(1-naphthyl)thiazol-4-ylcarbonyl]hydrazide;
N-(N-benzyloxycarbonyl-L-b-cyclopropylalanyl)-N′-[2-(1-naphthyl)thiazol-4-ylcarbonyl]hydrazide;
N-[2-(1-naphthyl)thiazol-4-ylcarbonyl]-N′-[N-[3-(2-pyridyl)phenylacetyl]-L-leucinyl]hydrazide;
N-[2-[N-bis-(cyclopropylmethyl)amino]thiazol-4-ylcarbonyl]-N′-(N-picolinyl-L-leucinyl)hydrazide;
N-(N-benzyloxycarbonyl-L-leucinyl)-N′-[2-[N-bis-(cyclopropylmethyl)amino]thiazol-4-ylcarbonyl]hydrazide;
N-[2-(N-cyclopropyl-N-cyclopropylmethylamino)thiazol-4-ylcarbonyl]-N′-[N-(6-methylnicotinoyl)-L-leucinyl]hydrazide;
N-[2-(N-cyclopropyl-N-cyclopropylmethylamino)thiazol-4-ylcarbonyl]-N′-[N-(3-methylisonicotinoyl)-L-leucinyl]hydrazide;
N-[2-(N-cyclopropyl-N-cyclopropylmethylamino)thiazol-4-ylcarbonyl]-N′-[N-(8-quinolinoyl)-L-leucinyl]hydrazide;
N-[2-[N-bis-(cyclopropylmethyl)amino]thiazol-4-ylcarbonyl]-N′-[N-(8-quinolinoyl)-L-leucinyl]hydrazide;
N-[2-[N-bis-(cyclopropylmethyl)amino]thiazol-4-ylcarbonyl]-N′-[N-(3-isoquinolinoyl)-L-leucinyl]hydrazide;
N-[2-[4-(2,2-dimethylaminoethoxy)-1-naphthyl]thiazol-4-ylcarbonyl]-N′-[N-(8-quinolinoyl)-L-leucinyl]hydrazide;
N-[2(N-cyclopropyl-N-cyclopropylmethylamino)thiazol-4-ylcarbonyl]-N′-[N-(7-quinolinoyl)-L-leucinyl]hydrazide;
N-[2-[N-bis-(cyclopropylmethyl)amino]thiazol-4-ylcarbonyl]-N′-[N-(6-methylnicotinoyl)-L-leucinyl]hydrazide;
N-[2-[N-bis-(cyclopropylmethyl)amino]thiazol-4-ylcarbonyl]-N′-(N-methyl-L-prolinyl-L-leucinyl)hydrazide;
N-(N-benzyloxycarbonyl-L-norvalinyl)-N′-[2-(1-naphthyl)thiazol-4-ylcarbonyl]hydrazide;
N-(N-benzyloxycarbonyl-L-isoleucinyl)-N′-[2-(1-naphthyl)thiazol-4-ylcarbonyl]hydrazide;
N-(N-benzyloxycarbonyl-L-norleucinyl)-N′-[2-(1-naphthyl)thiazol-4-ylcarbonyl]hydrazide;
N-[N-(4-dimethylaminomethylbenzyloxycarbonyl)-L-leucinyl]-N′-[2-(1-naphthyl)thiazol-4-ylcarbonyl]hydrazide;
N-(N-benzyloxycarbonyl-L-norvalinyl)-N′-[2-(2-benzyloxyphenyl)thiazol-4-ylcarbonyl]hydrazide;
N-[2-(N-cyclopropyl-N-cyclopropylmethylamino)thiazol-4-ylcarbonyl]N′-[N-(4-methylimidazol-5-ylcarbonyl)-L-leucinyl]hydrazide;
N-[N-[4-(4-morpholinomethyl)benzoyl]-L-leucinyl]-N′-[2-(1-naphthyl)thiazol-4-ylcarbonyl]hydrazide;
N-[N-(6-methylnicotinoyl)-L-leucinyl]-N′-[2-(1-naphthyl)thiazol-4-ylcarbonyl]hydrazide;
N-(N-b-tert-butoxycarbonyl-L-tert-butylalanyl)-N′-[2-(N-cyclopropyl-N-cyclopropylmethylamino)thiazol-4-ylcarbonyl]hydrazide;
N-[2-(N-cyclopropyl-N-cyclopropylmethylamino)thiazol-4-ylcarbonyl]-N′-[N-(8-quinolinoyl)-L-b-tert-butylalanyl]hydrazide;
N-[N-(4-methylimidazol-5-ylcarbonyl)-L-allylglycinyl]-N′-[2-(1-naphthyl)thiazol-4-ylcarbonyl]hydrazide;
N-[N-(4-methylimidazol-5-ylcarbonyl)-L-b-tert-butylalanyl]-N′-[2-(1-naphthyl)thiazol-4-ylcarbonyl]hydrazide;
N-[2-(N-cyclopropyl-N-cyclopropylmethylamino)thiazol-4-ylcarbonyl]-N′-[N-(4-methylimidazol-5-ylcarbonyl)-L-b-tert-butylalanyl]hydrazide;
N-[2-(1-naphthyl)thiazol-4-ylcarbonyl]-N′-(N-picolinoyl-L-b-tert-butylalanyl)hydrazide;
N-[2-(1-naphthyl)thiazol-4-ylcarbonyl]-N′-[N-(8-quinolinoyl)-L-b-tert-butylalanyl]hydrazide;
N-[2-(1-naphthyl)thiazol-4-ylcarbonyl]-N′-(N-picolinoyl-L-allylglycinyl)hydrazide;
N-[2-(1-naphthyl)thiazol-4-ylcarbonyl]-N′-(N-picolinoyl-L-b-cyclopropylalanyl)hydrazide;
N-[N-(6-methylnicotinoyl)-L-b-cyclopropylalanyl]-N′-[2-(1-naphthyl)thiazol-4-ylcarbonyl]hydrazide;
N-[N-(4-methylimidazol-5-ylcarbonyl)-L-b-cyclopropylalanyl]-N′-[2-(1-naphthyl)thiazol-4-ylcarbonyl]hydrazide;
N-[2-(1-naphthyl)thiazol-4-ylcarbonyl]-N′-[N-(8-quinolinoyl)-L-b-cyclopropylalanyl]hydrazide;
N-[N-(6-methylnicotinoyl)-L-b-tert-butylalanyl]-N′-[2-(1-naphthyl)thiazol-4-ylcarbonyl]hydrazide;
N-[2-(N-cyclopropyl-N-cyclopropylmethylamino)thiazol-4-ylcarbonyl]-N′-(N-picolinoyl-L-b-tert-butylalanyl)hydrazide;
N-[2-(N-cyclopropyl-N-cyclopropylmethylamino)thiazol-4-ylcarbonyl]-N′-[N-(3-isoquinolinoyl)-L-b-tert-butylalanyl]hydrazide;
N-(N-tert-butoxycarbonyl-L-b-cyclopropylalanyl)-N′-[2-(N-cyclopropyl-N-cyclopropylmethylamino)thiazol-4-ylcarbonyl]hydrazide;
N-[2-(N-cyclopropylmethyl-N-propylamino)thiazol-4-ylcarbonyl]-N′-[N-(6-methyl-3-pyridinylmethoxycarbonyl)-L-leucinyl]hydrazide;
N-[N-(6-methylnicotinoyl)-L-allylglycinyl]-N′-[2-(1-naphthyl)thiazol-4-ylcarbonyl]hydrazide;
N-[2-(1-naphthyl)thiazol-4-ylcarbonyl]-N′-[N-(8-quinolinoyl)-L-allylglycinyl]hydrazide;
N-[2-(1-naphthyl)thiazol-4-ylcarbonyl]-N′-[N-(2-quinolinoyl)-L-b-cyclopropylalanyl]hydrazide;
N-[N-(3-isoquinolinoyl)-L-b-cyclopropylalanyl]-N′[-[2-(1-naphthyl)thiazol-4-ylcarbonyl]hydrazide;
N-[N-(1-isoquinolinoyl)-L-b-cyclopropylalanyl]-N′-[2-(1-naphthyl)thiazol-4-ylcarbonyl]hydrazide;
N-[2-(1-naphthyl)thiazol-4-ylcarbonyl]-N′-[N-(7-quinolinoyl)-L-b-cyclopropylalanyl]hydrazide;
N-[2-(N-cyclopropyl-N-cyclopropylmethylamino)thiazol-4-ylcarbonyl]-N′-[N-(8-quinolinoyl)-L-b-cyclopropylalanyl]hydrazide;
N-[2-(N-cyclopropyl-N-cyclopropylmethylamino)thiazol-4-ylcarbonyl]-N′-[N-(4-methylimidazol-5-ylcarbonyl)-L-b-cyclopropylalanyl]hydrazide;
N-[2-(N-cyclopropyl-N-cyclopropylmethylamino)thiazol-4-ylcarbonyl]-N′-[N-(3-isoquinolinoyl)-L-b-cyclopropylalanyl]hydrazide;
N-[2-(N-cyclopropyl-N-cyclopropylmethylamino)thiazol-4-ylcarbonyl]-N′-[N-(6-methylnicotinoyl)-L-b-cyclopropylalanyl]hydrazide;
N-[N-(4-methylimidazol-5-ylcarbonyl)-L-norleucinyl]-N′-[2-(1-naphthyl)thiazol-4-ylcarbonyl]hydrazide;
N-[2-(1-naphthyl)thiazol-4-ylcarbonyl]-N′-(N-picolinoyl-L-norleucinyl]hydrazide;
N-[2-(1-naphthyl)thiazol-4-ylcarbonyl]-N′-[N-(8-quinolinoyl)-L-norleucinyl]hydrazide;
N-[2-(N-cyclopropyl-N-cyclopropylmethylamino)thiazol-4-ylcarbonyl]-N′-[N-(2-quinolinoyl)-L-b-cyclopropylalanyl]hydrazide;
N-[2-(N-cyclopropyl-N-cyclopropylmethylamino)thiazol-4-ylcarbonyl]-N′-[N-(1-isoquinolinoyl)-L-b-cyclopropylalanyl]hydrazide;
N-[2-[N-cyclopropyl-N-(2-methylpropyl)amino]thiazol-4-ylcarbonyl]-N′-[N-(6-methyl-3-pyridinylmethoxycarbonyl)-L-leucinyl]hydrazide;
N-(N-tert-butoxycarbonyl-L-leucinyl)-N′-[2-[N-cyclopropyl-N-(2-methylpropyl)amino]thiazol-4-ylcarbonyl]hydrazide;
N-[2-(1-naphthyl)thiazol-4-ylcarbonyl]-N′-[N-(7-quinolinoyl)-L-b-tert-butylalanyl]hydrazide;
N-[2-(1-naphthyl)thiazol-4ylcarbonyl]-N′-[N-(2-quinolinoyl)-L-b-tert-butylalanyl]hydrazide;
N-[N-(1-isoquinolinoyl)-L-b-tert-butylalanyl]-N′-[2-(1-naphthyl)thiazol-4-ylcarbonyl]hydrazide;
N-[N-(3-isoquinolinoyl)-L-b-tert-butylalanyl]-N′-[2-(1-naphthyl)thiazol-4-ylcarbonyl]hydrazide;
N-[N-(6-methylnicotinoyl)-L-norleucinyl]-N′-[2-(1-naphthyl)thiazol-4-ylcarbonyl]hydrazide;
N-[2-(1-naphthyl)thiazol-4-ylcarbonyl]-N′-[N-(7-quinolinoyl)-L-norleucinyl]hydrazide,;
N-[2-(1-naphthyl)thiazol-4-ylcarbonyl]-N′-[N-(2-quinolinoyl)-L-norleucinyl]hydrazide;
N-[N-(1-isoquinolinoyl)-L-norleucinyl]-N′-[2-(1-naphthyl)thiazol-4-ylcarbonyl]hydrazide;
N-[N-(3-isoquinolinoyl)-L-norleucinyl]-N′-[2-(1-naphthyl)thiazol-4-ylcarbonyl]hydrazide;
N-[2-(N-cyclopropyl-N-cyclopropylmethylamino)thiazol-4-ylcarbonyl]-N′-[N-(5-hydroxymethylimidazol-4-ylcarbonyl)-L-b-cyclopropylalanyl]hydrazide;
N-[2-[N-cyclopropyl-N-(2-methylpropyl)amino]thiazol-4-ylcarbonyl]-N′-[N-(8-quinolinoyl)-L-b-cyclopropylalanyl]hydrazide;
N-[2-(N-cyclopropyl-N-cyclopropylmethylamino)thiazol-4-ylcarbonyl]-N′-(N-(6-methylnicotinoyl)-L-b-tert-butylalanyl]hydrazide;
N-[2-[N-cyclopropyl-N-(2-methylpropyl)amino]thiazol-4-ylcarbonyl]-N′-[N-(4-methylimidazol-5-ylcarbonyl)-L-b-cyclopropylalanyl]hydrazide;
N-[2-[N-cyclopropyl-N-(2-methylpropyl)amino]thiazol-4-ylcarbonyl]-N′-[N-(2-quinolinoyl)-L-b-cyclopropylalanyl]hydrazide;
N-[2-[N-cyclopropyl-N-(2-methylpropyl)amino]thiazol-4-ylcarbonyl]-N′-[N-(6-methylnicotinoyl)-L-b-cyclopropylalanyl]hydrazide;
N-[2-(1-naphthyl)thiazol-4-ylcarbonyl]-N′-[N-(8-quinolinoyl)glycinyl]hydrazide;
N-[2-(1-naphthyl)thiazol-4-ylcarbonyl]-N′-[N-(8-quinolinoyl)-L-norvalinyl]hydrazide;
N-[2-(1-naphthyl)thiazol-4-ylcarbonyl]-N′-[N-(2-quinolinoyl)-L-norvalinyl]hydrazide;
N-[2-(1-naphthyl)thiazol-4-ylcarbonyl]-N′-(N-picolinoyl-L-norvalinyl]hydrazide;
N-[2-(1-naphthyl)thiazol-4-ylcarbonyl]-N′-[N-(6-methylnicotinoyl)-L-norvalinyl]hydrazide;
N-[2-(1-naphthyl)thiazol-4-ylcarbonyl]-N′-[N-(4-methylimidazol-5-ylcarbonyl)-L-norvalinyl]hydrazide;
N-[2-(1-naphthyl)thiazol-4-ylcarbonyl]-N′-[N-(1-isoquinolinoyl)-L-norvalinyl]hydrazide;
N-[2-(1-naphthyl)thiazol-4-ylcarbonyl]-N′-[N-(3-isoquinolinoyl)-L-norvalinyl]hydrazide;
(1S, 1′S)-N,N′-bis-[4-[1-(N-benzyloxycarbonylamino)-3-methylbutyl]thiazol-2-ylcarbonyl]hydrazide;
N-[2-[N-cyclopropyl-N-(2-methylpropyl)amino]thiazol-4-ylcarbonyl]-N′-[N-(6-methylnicotinoyl)-L-b-tert-butylalanyl]hydrazide;
N-[2-[N-cyclopropyl-N-(2-methylpropyl)amino]thiazol-4-ylcarbonyl]-N′-[N-(4-methylimidazol-5-ylcarbonyl)-L-b-tert-butylalanyl]hydrazide;
N-[2-(N-cyclopropyl-N-cyclopropylmethylamino)thiazol-4-ylcarbonyl]-N′-[N-(1-isoquinolinoyl)-L-b-tert-butylalanyl]hydrazide;
N-[N-(5-butylpicolinoyl)-L-b-tert-butylalanyl]-N′-[2-(N-cyclopropyl-N-cyclopropylmethylamino)thiazol-4-ylcarbonyl]hydrazide;
N-[2-(N-cyclopropyl-N-cyclopropylmethylamino)thiazol-4-ylcarbonyl]-N′-[N-(6-methylpicolinoyl)-L-b-tert-butylalanyl]hydrazide;
N-[2-(N-cyclopropyl-N-cyclopropylmethylamino)thiazol-4-ylcarbonyl]-N′-[N-(4-fluorobenzoyl)-L-leucinyl]hydrazide;
N-[N-(4-fluorobenzoyl)-L-leucinyl]-N′-[2-(1-naphthyl)thiazol-4-ylcarbonyl]hydrazide;
N-[2-(1-naphthyl)thiazol-4-ylcarbonyl]-N′-[N-(2-pyridinylmethoxycarbonyl)-L-b-tert-butylalanyl]hydrazide;
N-[N-(2-methyl-3-pyridinylmethoxycarbonyl)-L-b-tert-butylalanyl]-N′-[2-(1-naphthyl)thiazol-4-ylcarbonyl]hydrazide;
N-[2-(1-naphthyl)thiazol-4-ylcarbonyl]-N′-[N-(2-pyridinylmethoxycarbonyl)-L-b-cyclopropylalanyl]hydrazide;
N-[N-(2-methyl-3-pyridinylmethoxycarbonyl)-L-b-cyclopropylalanyl]-N′-[2-(1-naphthyl)thiazol-4-ylcarbonyl]hydrazide;
N-[N-(6-methyl-3-pyridinylmethoxycarbonyl)-L-b-cyclopropylalanyl]-N′-[2-(1-naphthyl)thiazol-4-ylcarbonyl]hydrazide;
N-[N-(6-methyl-3-pyridinylmethoxycarbonyl)-L-b-tert-butylalanyl]-N′-[2-(1-naphthyl)thiazol-4-ylcarbonyl]hydrazide;
N,N′-bis-[2-(1-naphthyl)thiazol-4-ylcarbonyl]hydrazide;
N-[2-(N-cyclopropyl-N-cyclopropylmethylamino)thiazol-4-ylcarbonyl]-N′-[N-[2-(1,8-naphthyridinoyl))-L-b-cyclopropylalanyl]hydrazide;
N-[2-[N-cyclopropyl-N-(2-methylpropyl)amino]thiazol-4-ylcarbonyl]-N′-[N-(3,4-difluorobenzoyl)-L-b-cyclopropylalanyl]hydrazide;
N-[2-[N-cyclopropyl-N-(2-methylpropyl)amino]thiazol-4-ylcarbonyl]-N′-[N-(4-flluorobenzoyl)-L-leucinyl]hydrazide;
N-[N-(5-butylpicolinoyl)-L-leucinyl]-N′-[2-(N-cyclopropyl-N-cyclopropylmethylamino)thiazol-4-ylcarbonyl]hydrazide;
N-[2-[N-cyclopropyl-N-(2-methylpropyl)amino]thiazol-4-ylcarbonyl]-N′-[N-(3,4-dimethoxybenzoyl)-L-leucinyl]hydrazide;
N-[2-[N-cyclopropyl-N-(2-methylpropyl)amino]thiazol-4-ylcarbonyl]-N′-[N-(3,4-difluorobenzoyl)-L-b-tert-butylalanyl]hydrazide;
N-[2-[N-cyclopropyl-N-(2-methylpropyl)amino]thiazol-4-ylcarbonyl]-N′-[N-(3,4-dimethoxybenzoyl)-L-b-tert-butylalanyl]hydrazide;
N-[N-(5-butylpicolinoyl)-L-b-tert-butylalanyl]-N′-[2-(N-cyclopropyl-N-cyclopropylmethylamino)thiazol-4-ylcarbonyl]hydrazide; and
N-[2-[N-cyclopropyl-N-(2-methylpropyl)amino]thiazol-4-ylcarbonyl]-N′-[N-(6-methylpicolinoyl)-L-b-tert-butylalanyl]hydrazide.

22. A pharmaceutical composition comprising a compound according to claim 1 and a pharmaceutically acceptable carrier, diluent or excipient.

23. A pharmaceutical composition comprising a compound according to claim 21 and a pharmaceutically acceptable carrier, diluent or excipient.

24. A method of inhibiting a protease selected from the group consisting of a cysteine protease and a serine protease, comprising administering to a patient in need thereof an effective amount of a compound according to claim 1.

25. A method of inhibiting a protease selected from the group consisting of a cysteine protease and a serine protease, comprising administering to a patient in need thereof an effective amount of a compound according to claim 21.

26. A method according to claim 24 wherein said protease is a cysteine protease.

27. A method according to claim 25 wherein said protease is a cysteine protease.

28. A method according to claim 26 wherein said cysteine protease is cathepsin K.

29. A method according to claim 27 wherein said cysteine protease is cathepsin K.

30. A method of treating a disease characterized by bone loss comprising inhibiting said bone loss by administering to a patient in need thereof an effective amount of a compound according to claim 1.

31. A method according to claim 30 wherein said disease is osteoporosis.

32. A method according to claim 30 wherein said disease is periodontitis.

33. A method according to claim 30 wherein said disease is gingivitis.

34. A method of treating a disease characterized by excessive cartilage or matrix degradation comprising inhibiting said excessive cartilage or matrix degradation by administering to a patient in need thereof an effective amount of a compound according to claim 1.

35. A method according to claim 34 wherein said disease is osteoarthritis

36. A method according to claim 34 wherein said disease is rheumatoid arthritis.

37. A method of treating a disease characterized by bone loss comprising inhibiting said bone loss by administering to a patient in need thereof an effective amount of a compound according to claim 21.

38. A method according to claim 37 wherein said disease is osteoporosis.

39. A method according to claim 37 wherein said disease is periodontitis.

40. A method according to claim 37 wherein said disease is gingivitis.

41. A method of treating a disease characterized by excessive cartilage or matrix degradation comprising inhibiting said excessive cartilage or matrix degradation by administering to a patient in need thereof an effective amount of a compound according to claim 21.

42. A method according to claim 41 wherein said disease is osteoarthritis.

43. A method according to claim 41 wherein said disease is rheumatoid arthritis.

44. A method for preparing compounds according to claim 1, comprising the step of reacting an intermediate: 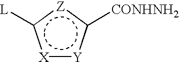
with a carboxylic acid, R³CO₂H, and a peptide coupling reagent in an aprotic solvent.

45. A method according to claim 44 wherein said peptide coupling reagent is EDC.HCl/1-HOBT when a carboxylic acid is used.

46. A method according to claim 45 wherein said aprotic solvent is DMF.

47. A method for preparing compounds according to claim 1, comprising the step of reacting an intermediate: 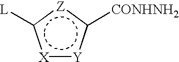
with a carbamoyl chloride, R³COCl, and triethylamine in methylene chloride.

48. A method for preparing compounds according to claim 1, comprising the step of reacting an intermediate: 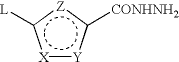
with a sulfonyl chloride, R³SO₂Cl, and NMM in CH₂Cl₂.

49. Use of a compound according to any one of claims 1 to 21 in the manufacture of a medicament for use in inhibiting a protease selected from the group consisting of a cysteine protease and a serine protease.

50. A use according to claim 49 wherein said protease is a cysteine protease.

51. A use according to claim 51 wherein said cysteine protease is cathepsin K.

52. Use of a compound according to any one of claims 1 to 21 in the manufacture of a medicament for use in treating a disease characterized by bone loss.

53. A use according to claim 52 wherein said disease is osteoporosis.

54. A use according to claim 52 wherein said disease is periodontitis.

55. A use according to claim 52 wherein said disease is gingivitis.

56. Use of a compound according to any one of claims 1 to 21 in the manufacture of a medicament for use in treating a disease characterized by excessive cartilage or matrix degradation.

57. A use according to claim 56 wherein said disease is osteoarthritis.

58. A use according to claim 56 wherein said disease is rheumatoid arthritis.
